SEQUENCE LISTING (60 sequences)

SEQ ID NO: 1 - protein, 479 aa, synthetic construct
MEPAAALHFSLPASLLLLLLLLLLSLCALVSAQFTVVGPANPILAMVGENTTLRCHLSPEKNAEDMEVRWFRSQFSPAVFVYKGGRERTEEQMEEYRGRITFVSKDINRGSVALVIHNVTAQENGIYRCYFQEGRSYDEAILRLVVAGLGSKPLIEIKAQEDGSIWLECISGGWYPEPLTVWRDPYGEVVPALKEVSIADADGLFMVTTAVIIRDKYVRNVSCSVNNTLLGQEKETVIEGRMDDISSTMVRSDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 2 - protein, 466 aa, synthetic construct
MEPAAALHFSLPASLLLLLLLLLLSLCALVSAQFTVVGPANPILAMVGENTTLRCHLSPEKNAEDMEVRWFRSQFSPAVFVYKGGRERTEEQMEEYRGRITFVSKDINRGSVALVIHNVTAQENGIYRCYFQEGRSYDEAILRLVVAGLGSKPLIEIKAQEDGSIWLECISGGWYPEPLTVWRDPYGEVVPALKEVSIADADGLFMVTTAVIIRDKYVRNVSCSVNNTLLGQEKETVRSDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVLHEALHSHYTQKSLSLSPGK

SEQ ID NO: 3 - protein, 237 aa, Homo sapiens
MEPAAALHFSLPASLLLLLLLLLLSLCALVSAQFTVVGPANPILAMVGENTTLRCHLSPEKNAEDMEVRWFRSQFSPAVFVYKGGRERTEEQMEEYRGRITFVSKDINRGSVALVIHNVTAQENGIYRCYFQEGRSYDEAILRLVVAGLGSKPLIEIKAQEDGSIWLECISGGWYPEPLTVWRDPYGEVVPALKEVSIADADGLFMVTTAVIIRDKYVRNVSCSVNNTLLGQEKETV

SEQ ID NO: 4 - protein, 227 aa, Homo sapiens
DKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 5 - protein, 227 aa, synthetic construct
DKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVLHEALHSHYTQKSLSLSPGK

SEQ ID NO: 6 - protein, 5 aa, synthetic construct
GGGGS

SEQ ID NO: 7 - protein, 6 aa, synthetic construct
GGGGGS

SEQ ID NO: 8 - DNA, 29 bp, synthetic construct
  source 1..29: mol_type = other DNA
atataccggtatggaaccagctgctgctc

SEQ ID NO: 9 - DNA, 49 bp, synthetic construct
  source 1..49: mol_type = other DNA
atatgatatcgtccatacgtccctcgatgacagtttccttctcctggcc

SEQ ID NO: 10 - DNA, 29 bp, synthetic construct
  source 1..29: mol_type = other DNA
atattctagaatggaaccagctgctgctc

SEQ ID NO: 11 - DNA, 30 bp, synthetic construct
  source 1..30: mol_type = other DNA
atatgagctcactcatttacccggagacag

SEQ ID NO: 12 - DNA, 20 bp, synthetic construct
  source 1..20: mol_type = other DNA
accacagtccatgccatcac

SEQ ID NO: 13 - DNA, 20 bp, synthetic construct
  source 1..20: mol_type = other DNA
tccaccaccctgttgctgta

SEQ ID NO: 14 - DNA, 20 bp, synthetic construct
  source 1..20: mol_type = other DNA
cgccaccagttcgccatgga

SEQ ID NO: 15 - DNA, 20 bp, synthetic construct
  source 1..20: mol_type = other DNA
tacagcccggggagcatcgt

SEQ ID NO: 16 - DNA, 22 bp, synthetic construct
  source 1..22: mol_type = other DNA
cctggttgtgagaaggtcttcg

SEQ ID NO: 17 - DNA, 22 bp, synthetic construct
  source 1..22: mol_type = other DNA
tgctccagagactgcaccactt

SEQ ID NO: 18 - DNA, 18 bp, synthetic construct
  source 1..18: mol_type = other DNA
cacggccctggttctcat

SEQ ID NO: 19 - DNA, 24 bp, synthetic construct
  source 1..24: mol_type = other DNA
gcagatgttccactctcctcttct

SEQ ID NO: 20 - DNA, 25 bp, synthetic construct
  source 1..25: mol_type = other DNA
aaagatggggagagaaagtcaagag

SEQ ID NO: 21 - DNA, 24 bp, synthetic construct
  source 1..24: mol_type = other DNA
cctacctagttttacctgaggttc

SEQ ID NO: 22 - DNA, 24 bp, synthetic construct
  source 1..24: mol_type = other DNA
ggtccattgatatccaaagccaaa

SEQ ID NO: 23 - DNA, 22 bp, synthetic construct
  source 1..22: mol_type = other DNA
ctcaagtgccacatcccagatc

SEQ ID NO: 24 - DNA, 22 bp, synthetic construct
  source 1..22: mol_type = other DNA
agcacttccagcggagagatct

SEQ ID NO: 25 - DNA, 23 bp, synthetic construct
  source 1..23: mol_type = other DNA
gcctcctgattagacttcgtcac

SEQ ID NO: 26 - DNA, 22 bp, synthetic construct
  source 1..22: mol_type = other DNA
caggcaaaagctgcatgctcac

SEQ ID NO: 27 - DNA, 22 bp, synthetic construct
  source 1..22: mol_type = other DNA
cctctggaggaggaacgctaat

SEQ ID NO: 28 - DNA, 22 bp, synthetic construct
  source 1..22: mol_type = other DNA
gtttcgggtctggatgccttct

SEQ ID NO: 29 - DNA, 22 bp, synthetic construct
  source 1..22: mol_type = other DNA
ccacctgttgtggtccaagttc

SEQ ID NO: 30 - DNA, 24 bp, synthetic construct
  source 1..24: mol_type = other DNA
ccacaaacatcctgtaatggcttg

SEQ ID NO: 31 - DNA, 22 bp, synthetic construct
  source 1..22: mol_type = other DNA
gctttgaggctgtctaccagct

SEQ ID NO: 32 - DNA, 22 bp, synthetic construct
  source 1..22: mol_type = other DNA
gtgaggaccttgtcaagccact

SEQ ID NO: 33 - DNA, 22 bp, synthetic construct
  source 1..22: mol_type = other DNA
agaaccaggtcttctgccaagg

SEQ ID NO: 34 - DNA, 22 bp, synthetic construct
  source 1..22: mol_type = other DNA
ttgtgggtggaggtgtcgttct

SEQ ID NO: 35 - DNA, 24 bp, synthetic construct
  source 1..24: mol_type = other DNA
caccggtttgcagaattaccacaa

SEQ ID NO: 36 - DNA, 24 bp, synthetic construct
  source 1..24: mol_type = other DNA
aaacttgtggtaattctgcaaacc

SEQ ID NO: 37 - DNA, 24 bp, synthetic construct
  source 1..24: mol_type = other DNA
caccgtaatatttacccaccaccc

SEQ ID NO: 38 - DNA, 24 bp, synthetic construct
  source 1..24: mol_type = other DNA
aaacgggtggtgggtaaatattac

SEQ ID NO: 39 - DNA, 23 bp, synthetic construct
  source 1..23: mol_type = other DNA
aggctatcgggcaagaacgcagg

SEQ ID NO: 40 - DNA, 23 bp, synthetic construct
  source 1..23: mol_type = other DNA
aaatccgattcatgtggacctgg

SEQ ID NO: 41 - DNA, 23 bp, synthetic construct
  source 1..23: mol_type = other DNA
gtcatgtacatgagaaccgtggg

SEQ ID NO: 42 - DNA, 23 bp, synthetic construct
  source 1..23: mol_type = other DNA
tagtgagacctatcttgaagggg

SEQ ID NO: 43 - DNA, 24 bp, synthetic construct
  source 1..24: mol_type = other DNA
cctacctagttttacctgaggttc

SEQ ID NO: 44 - DNA, 24 bp, synthetic construct
  source 1..24: mol_type = other DNA
ggtccattgatatccaaagccaaa

SEQ ID NO: 45 - DNA, 24 bp, synthetic construct
  source 1..24: mol_type = other DNA
aagatggggagagaaagtcaagag

SEQ ID NO: 46 - DNA, 36 bp, synthetic construct
  source 1..36: mol_type = other DNA
gggtttgcagaattaccatggtgggtaaatattaca

SEQ ID NO: 47 - protein, 12 aa, synthetic construct
GFAELPWWVNIT

SEQ ID NO: 48 - DNA, 22 bp, synthetic construct
  source 1..22: mol_type = other DNA
tttcataattttaccctctgtt

SEQ ID NO: 49 - protein, 8 aa, synthetic construct
ISAFYPLL

SEQ ID NO: 50 - DNA, 20 bp, synthetic construct
  source 1..20: mol_type = other DNA
aaaagtgcaacgtaatggaa

SEQ ID NO: 51 - protein, 7 aa, synthetic construct
KSATAWK

SEQ ID NO: 52 - DNA, 24 bp, synthetic construct
  source 1..24: mol_type = other DNA
tttcataggcttttaccctctntt

SEQ ID NO: 53 - protein, 7 aa, synthetic construct
FHRLLPS

SEQ ID NO: 54 - DNA, 20 bp, synthetic construct
  source 1..20: mol_type = other DNA
aaaagtgcaacgtgctggaa

SEQ ID NO: 55 - protein, 7 aa, synthetic construct
QKCNVLE

SEQ ID NO: 56 - protein, 15 aa, synthetic construct
IEGRMDDISSTMVRS

SEQ ID NO: 57 - protein, 6 aa, synthetic construct
IEGRMD

SEQ ID NO: 58 - protein, 7 aa, synthetic construct
EAEAEAK

SEQ ID NO: 59 - protein, 8 aa, synthetic construct
SIINFEKL

SEQ ID NO: 60 - protein, 4 aa, synthetic construct
GGGS

(19) United States
(12) Patent Application Publication
(10) Pub. No.: US 2026/0242441 A1
(43) Pub. Date: Aug. 20, 2026

(54) BUTYROPHILIN A2 AND RELATED ISOFORMS FOR THE TREATMENT OF AUTOIMMUNITY AND INFLAMMATION

(71) Applicant: CEDARS-SINAI MEDICAL CENTER, Los Angeles, CA (US)

(72) Inventors: S. Ananth KARUMANCHI, Los Angeles, CA (US); Stanley JORDAN, Manhattan Beach, CA (US); Shafat ALI, Baldwin Park, CA (US); Anders H. BERG, Culver City, CA (US); Ravi THADHANI, Boston, MA (US); Ruan ZHANG, Winchester, MA (US); Michifumi YAMASHITA, Beverly Hills, CA (US)

(21) Appl. No.: 19/161,729

(22) PCT Filed: Mar. 1, 2024
(86) PCT No.: PCT/US2024/018076

§ 371 Date: Sep. 3, 2025

Related U.S. Application Data
(60) Provisional application No. 63/449,693, filed on Mar. 3, 2023.

(51) Int. Cl. C07K 14/705; A61K 9/00; A61K 38/17; A61K 47/68; A61P 29/00; G01N 33/543; G01N 33/68

(52) U.S. Cl. CPC C07K 14/70503; A61K 9/0019; A61K 38/1774; A61K 47/6811; A61P 29/00; G01N 33/543; G01N 33/6896; C07K 2319/30; G01N 2333/70503; G01N 2800/52; G01N 2800/56

(57) ABSTRACT

Described herein are methods of reducing CD3-dependent T cell signaling in a subject in need thereof. Also described are method of increasing T-regulatory (Treg) cells, or decreasing T-helper 17 (Th17) cells. These methods involve administering butyrophilin A2 (BTN2A2), a BTN2A2 fragment thereof, a BTN2A2-related isoform, or a BTN2A2-related isoform fragment, or a conjugate or fusion polypeptide comprising any of the foregoing to the subject. These methods are beneficial for patients with autoimmune disorders and inflammatory disorders such as allergy, asthma, glomerulonephritis, inflammatory bowel disease, rheumatoid arthritis, an autoimmune or inflammatory neurological disease, antibody mediated transplant rejection, infantile cholestasis, haemophagocytic lymphohistiocytosis, erythrocytic haemophagocytosis, malnutrition, systemic lupus erythematosus (lupus), psoriasis, myasthenia gravis or HIV. Further described are fusion proteins having BTN2A2 and an Fc domain.

### CROSS-REFERENCE TO RELATED APPLICATIONS

This application includes a claim of priority under 35 U.S.C. § 119(e) to U.S. provisional patent application No. 63/449,693, filed Mar. 3, 2023, the entirety of which is hereby incorporated by reference.

### REFERENCE TO SEQUENCE LISTING

This application contains a Sequence Listing submitted as a computer readable form named “065472_000918WOPT_SequenceListing.xml”, having a size in bytes of 55.203 bytes, and created on Feb. 28, 2024. The information contained in this computer readable form is hereby incorporated by reference in its entirety.

### FIELD OF INVENTION

This invention relates to butyrophilin A2, its isoforms and fragments and conjugates thereof for the treatment of autoimmune and inflammatory disorders.

### BACKGROUND

All publications herein are incorporated by reference to the same extent as if each individual publication or patent application was specifically and individually indicated to be incorporated by reference. The following description includes information that may be useful in understanding the present invention. It is not an admission that any of the information provided herein is prior art or relevant to the presently claimed invention, or that any publication specifically or implicitly referenced is prior art.

Currently treatment of autoimmunity is largely non-specific with steroids and drugs like cyclosporine and prograf. While there are some promise with newer agents such as CTLA4IG, there remains a need for additional drugs that can for example, synergize to improve autoimmunity and inflammation.

T cells recognize antigens presented through peptide: MHC complexes using surface expressed, heterodimeric abT cell receptors (TCRs). As the TCR lacks intrinsic kinase activity, signals initiated by TCR ligation involve recruitment of the Src family kinase Lek. which then phosphorylates immunoreceptor tyrosine-kinase-based motifs (ITAMs) within the TCR-associated CD3 z-chain. Lek also phosphorylates subsequently recruited Zap70 kinase, thereby propagating requisite downstream signals required for full T cell activation. Studies performed over the past 30 years showed T cell activation is controlled in part by cell surface expressed CD45.

CD45 is a transmembrane glycoprotein that contains an intracellular tyrosine phosphatase domain capable of dephosphorylating multiple TCR immunoreceptor tyrosine activation (ITAM) motifs. Differential splicing results in expression of multiple CD45 isoforms (i.e. RA. RB, RC, RO). Following TCR stimulation, CD45 is initially recruited to the supramolecular activation cluster (SMAC) but is then expelled, segregating it from the TCR. Evidence suggests that this segregation of CD45's phosphatase activity from the TCR is essential for Lck-initiated signal propagation that results in full T-cell activation. Conversely, retention of CD45 within the T cell immune synapse (IS) regulates the strength and duration of TCR activation, and perturbations of CD45 activity contribute to development of autoimmune disease. In humans, aberrant CD45 expression has been reported in diseases including infantile cholestasis, haemophagocytic lymphohistiocytosis or erythrocytic haemophagocytosis, malnutrition, systemic lupus erythematosus, rheumatoid arthritis, myasthenia gravis and HIV (Tchilian et al, “Altered CD45 expression and disease”, TRENDS in Immunology 27(3):146-153, March 2006). Also, therapeutic modulation of CD45 function reportedly has direct clinical applicability in organ transplantation, treatment of autoimmune disease or microglial activation associated with Alzheimer disease (AD) (A Rheinlsnder et al., “CD45 in human physiology and clinical medicine”. Immunology Letters, 196:22-32, April 2018). However, despite decades of work by multiple groups delineating these molecular mechanisms, it is not known how CD45 segregation versus retention during TCR activation may be regulated, or if ligands co-presented by antigen-presenting cells (APCs) play a role.

Butyrophilins (BTNs) are glycoproteins enriched in breast milk that have imprecisely understood immune-regulatory effects and are implicated in maintaining maternal-fetal tolerance. The mRNAs encoding for BTN and BTN-like molecules are widely expressed in lymphoid and non-lymphoid tissues. Butyrophilin immunoglobulin domains exhibit structural similarities to the B7 family of co-receptors, including B7-1/CD80, B7-2/CD86, ICOS-L and PD-L1, and Butyrophilin 2A2 (BTN2A2) was previously shown to be expressed by professional APCs including B cells, macrophages, and dendritic cells (DCs). Results of in vitro studies suggest that BTN2A2 can modulate T cell receptor (TCR) signaling and promote de novo Foxp3 expression. Mice genetically deficient in BTN2A2 exhibit impaired CD4+ regulatory T cell function, potentiated anti-tumor immunity, and augmented clinical manifestations of experimental autoimmune encephalomyelitis, all of which were attributable to deficiency of BTN2A2 in APCs. While these cumulative findings implicate a key immunoregulatory function for BTN2A2, the exact molecular mechanisms underlying these effects remain unclear.

### SUMMARY OF THE INVENTION

The following embodiments and aspects thereof are described and illustrated in conjunction with compositions and methods which are meant to be exemplary and illustrative, not limiting in scope.

Various embodiments provide for a method of reducing CD3-dependent T cell signaling in a subject in need thereof, comprising administering butyrophilin A2 (BTN2A2), fragment thereof, a BTN2A2-related isoform, a BTN2A2-related isoform fragment or a fusion polypeptide comprising any of the foregoing, to the subject.

Various embodiments provide for a method of increasing T-regulatory (Treg) cells, or decreasing T-helper 17 (Th17) cells. or both in a subject in need thereof, comprising: administering butyrophilin A2 (BTN2A2), fragment thereof, a BTN2A2-related isoform, a BTN2A2-related isoform fragment, or a fusion polypeptide comprising any of the foregoing, to the subject.

In various embodiments, the BTN2A2, fragment thereof, BTN2A2-related isoform, BTN2A2-related isoform fragment or fusion polypeptide can comprise human BTNA2 or a fragment or isoform thereof.

In various embodiments, the subject can be pregnant, and the method inhibits or reduces the likelihood of miscarriage of a fetus by increasing Treg cells. In various embodiments, the subject can have renal damage or a condition associated with renal damage. In various embodiments, the subject can be an organ, tissue or cell transplant recipient. In various embodiments, the method reduces the likelihood of organ, tissue or cell transplant rejection and/or an autoimmune or inflammatory reaction.

In various embodiments, the method reduces a need for an immunosuppressive therapy or reduces the amount of immunosuppressive therapy administered to the subject.

In various embodiments, the method can reduce or eliminate the otherwise toxic effects of the immunosuppressive therapy in the subject.

In various embodiments, the subject can have an autoimmune or inflammatory disorder or a disorder characterized by the accumulation of pathologic Th1/Th17 cells, e.g., Th1/Th17 Cell-Polarizing Myeloid Dendritic Cells.

In various embodiments, the autoimmune or inflammatory disorder or disorder characterized by the accumulation of pathologic Th1/Th17 cells can comprise allergy, asthma, glomerulonephritis, inflammatory bowel disease or Crohn's disease, rheumatoid arthritis, an autoimmune neurological disease, antibody mediated transplant rejection, infantile cholestasis, haemophagocytic lymphohistiocytosis, erythrocytic haemophagocytosis, malnutrition, systemic lupus erythematosus (lupus), sarcoidosis, psoriasis, myasthenia gravis or HIV. In various embodiments, the autoimmune neurological disease can be multiple sclerosis or myelin oligodendrocyte glycoprotein (MOG) antibody disease or Alzheimer's disease.

Various embodiments provide for a method of treating a disease or condition in a subject in need thereof, comprising: administering butyrophilin A2 (BTN2A2), a BTN2A2 fragment thereof, a BTN2A2-related isoform, or a BTN2A2-related isoform fragment, to the subject.

In various embodiments, disease or condition can be an autoimmune disorder or inflammatory disorder.

In various embodiments, the autoimmune or inflammatory disorder can comprise allergy, asthma, glomerulonephritis, inflammatory bowel disease or Crohn's disease, rheumatoid arthritis, an autoimmune neurological disease, antibody mediated transplant rejection, infantile cholestasis, haemophagocytic lymphohistiocytosis, erythrocytic haemophagocytosis, malnutrition, systemic lupus erythematosus (lupus), sarcoidosis, psoriasis, myasthenia gravis or HIV.

In various embodiments, the autoimmune neurological disease can be multiple sclerosis or myelin oligodendrocyte glycoprotein (MOG) antibody disease or Alzheimer's disease.

In various embodiments, the disease or condition can be associated with renal damage.

In various embodiments, disease or condition can be organ transplant rejection and the method reduces the likelihood of the organ transplant rejection.

In various embodiments, the disease or condition can be organ transplant rejection and the method reduces a need for an immunosuppressive therapy or reducing the amount of immunosuppressive therapy needed by the subject.

In various embodiments, the toxic effects of the immunosuppressive therapy can be reduced or eliminated in the subject.

In various embodiments, the butyrophilin A2 (BTN2A2) or the BTN2A2 fragment thereof can be linked to an albumin, optionally human serum albumin.

In various embodiments, the butyrophilin A2 (BTN2A2) or the BTN2A2 fragment thereof can be linked or fused to a Fc domain or a fragment of the Fc domain (“BTN2A2-Fc fusion protein”) or wherein the BTN2A2-related isoform or the BTN2A2-related isoform fragment is linked or fused to the Fc domain or the fragment of the Fc domain (“BTN2A2-related isoform-Fc fusion protein”). optionally an IgG1, IgG2, IgG3 or IgG4 Fe, which optionally may be mutated, further optionally mutated in order to enhance or reduce an Fe-associated effector function, further optionally any of the mutations shown in Table 3.

In various embodiments, the BTN2A2 or the BTN2A2 fragment thereof can be linked to a Fc domain or a fragment of the Fc domain by a linker, optionally a peptide of 2-50 amino acids, or wherein the BTN2A2-related isoform or the BTN2A2-related isoform fragment can be linked to the Fc domain or the fragment of the Fc domain by a linker, and the linker can be G, polyserine, polyglycine, glycine-serine, GGGGSn (SEQ ID NO:6)n. GGGGGSn (SEQ ID NO:7)n, leucine zipper. r aliphatic, or helical peptides.

In various embodiments, the butyrophilin A2 (BTN2A2) or the BTN2A2 fragment thereof, the BTN2A2-related isoform or the BTN2A2-related isoform fragment can be modified by glycosylation or PEGylation or lipidation or by attachment to cholesterol.

In various embodiments, the BTN2A2 fragment can comprise an extracellular domain of BTN2A2.

In various embodiments, the BTN2A2 fragment does not comprise a transmembrane domain, a cytoplasmic domain, or both.

In various embodiments, the BTN2A2-related isoform can be BTN1A1, B2N2A1, BTN3A1, BTN3A2 or BTN3A3.

In various embodiments, the BTN2A2-related isoform fragment does not comprise the transmembrane domain, the cytoplasmic domain, or both.

Various embodiments provide for fusion polypeptide, comprising: a butyrophilin A2 (BTN2A2), a BTN2A2 fragment, a BTN2A2-related isoform, or a BTN2A2-related isoform fragment or a combination thereof; and an Fc domain or a fragment of the Fc domain, optionally an IgG1, IgG2. IgG3 or IgG4 Fe, which optionally may be mutated, further optionally mutated in order to enhance or reduce an Fe-associated effector function, further optionally any of the mutations shown in Table 3.

In various embodiments, the fusion polypeptide can further comprise a linker between the BTN2A2, BTN2A2 fragment, BTN2A2-related isoform, BTN2A2-related isoform fragment, and the Fc domain or the fragment of the Fc domain, optionally an IgG1, IgG2. IgG3 or IgG4 Fe, which optionally may be mutated, further optionally mutated in order to enhance or reduce an Fe-associated effector function, further optionally any of the mutations shown in Table 3.

In various embodiments, the Fc domain or the fragment of the Fc domain can be from an IgG1 antibody.

In various embodiments, the Fc domain or the fragment of the Fc domain can comprise one or more mutations. In various embodiments, the mutation can be Met208Leu, Asn214Ser, or both, in reference to SEQ ID NO:4.

In various embodiments, the fusion polypeptide can further comprise a spacer peptide between the BTN2A2, BTN2A2 fragment, the BTN2A2-related isoform, or the BTN2A2-related isoform fragment, and the Fc domain or the fragment of the Fc domain.

In various embodiments, the spacer peptide can be selected from the group consisting of IEGRMDDISSTMVRS (SEQ ID NO:56), IEGRMD (SEQ ID NO:57). EAEAEAK (SEQ ID NO:58), SIINFEKL (SEQ ID NO:59), and GGGS (SEQ ID NO:60).

In various embodiments, the BTN2A2 fragment can comprise the extracellular domain of BTN2A2.

In various embodiments, the BTN2A2 fragment does not comprise the transmembrane domain, the cytoplasmic domain, or both.

In various embodiments, the BTN2A2-related isoform can be BTN1A1, B2N2A1, BTN3A1, BTN3A2 or BTN3A3.

In various embodiments, the BTN2A2-related isoform fragment does not comprise the transmembrane domain, the cytoplasmic domain, or both.

Various embodiments provide for a modified polypeptide, comprising: a butyrophilin A2 (BTN2A2), a BTN2A2 fragment, a BTN2A2-related isoform, or a BTN2A2-related isoform fragment or a combination thereof, wherein the butyrophilin A2 (BTN2A2), the BTN2A2 fragment, the BTN2A2-related isoform, or the BTN2A2-related isoform fragment, or the combination thereof is PEGylated or glycosylated.

In various embodiments, the BTN2A2 fragment can comprise the extracellular domain of BTN2A2.

In various embodiments, the BTN2A2 fragment does not comprise the transmembrane domain, the cytoplasmic domain, or both. In various embodiments, the BTN2A2 fragment can bind to CD45. e.g., CD45RO.

In various embodiments, the BTN2A2-related isoform can be BTN1A1, B2N2A1, BTN3A1, BTN3A2 or BTN3A3. In various embodiments, the BTN2A2-related isoform fragment does not comprise the transmembrane domain, the cytoplasmic domain, or both.

Various embodiments provide for an immunoassay, comprising: an anti-butyrophilin A2 (BTN2A2) antibody, an anti-BTN2A2 fragment antibody. an anti-BTN2A2-related isoform antibody, or an anti-BTN2A2-related isoform fragment antibody, or a combination thereof immobilized on a solid support; and the solid support.

In various embodiments, the immunoassay can further comprise a biological sample obtained from a subject.

Various embodiments provide for a method of using the assay of the present invention for patient monitoring, comprising: contacting a biological sample obtained from a patient to the assay of the present invention; and detecting the level of butyrophilin A2 (BTN2A2), the BTN2A2 fragment, the BTN2A2-related isoform, or the BTN2A2-related isoform fragment, or the combination thereof.

In various embodiments, the method can further comprise selecting or administrating a treatment selected from (i) butyrophilin A2 (BTN2A2), a BTN2A2 fragment thereof, a BTN2A2-related isoform, or a BTN2A2-related isoform fragment; (ii) a fusion polypeptide, comprising: a butyrophilin A2 (BTN2A2), a BTN2A2 fragment, a BTN2A2-related isoform. or a BTN2A2-related isoform fragment or a combination thereof; and an Fc domain or a fragment of the Fc domain; (iii) a modified polypeptide, comprising: a butyrophilin A2 (BTN2A2), a BTN2A2 fragment, a BTN2A2-related isoform, or a BTN2A2-related isoform fragment or a combination thereof, wherein the butyrophilin A2 (BTN2A2), the BTN2A2 fragment, the BTN2A2-related isoform, or the BTN2A2-related isoform fragment, or the combination thereof is PEGylated or glycosylated; or (iv) a non-BTN2A2 related treatment.

Various embodiments provide for a method of using the assay of the present invention for patient stratification, comprising: contacting a biological sample obtained from a patient to the assay of the present invention; detecting the level of butyrophilin A2 (BTN2A2), the BTN2A2 fragment, the BTN2A2-related isoform, or the BTN2A2-related isoform fragment, or the combination thereof, and stratifying the patient for treatment with (i) butyrophilin A2 (BTN2A2), a BTN2A2 fragment thereof, a BTN2A2-related isoform. or a BTN2A2-related isoform fragment; (ii) a fusion polypeptide, comprising: a butyrophilin A2 (BTN2A2), a BTN2A2 fragment, a BTN2A2-related isoform, or a BTN2A2-related isoform fragment or a combination thereof, and an Fc domain or a fragment of the Fc domain; (iii) a modified polypeptide, comprising: a butyrophilin A2 (BTN2A2), a BTN2A2 fragment, a BTN2A2-related isoform, or a BTN2A2-related isoform fragment or a combination thereof, wherein the butyrophilin A2 (BTN2A2), the BTN2A2 fragment, the BTN2A2-related isoform, or the BTN2A2-related isoform fragment, or the combination thereof is PEGylated or glycosylated; or (iv) a non-BTN2A2 related treatment.

Other features and advantages of the invention will become apparent from the following detailed description, taken in conjunction with the accompanying drawings, which illustrate, by way of example, various features of embodiments of the invention.

### BRIEF DESCRIPTION OF THE FIGURES

Exemplary embodiments are illustrated in referenced figures. It is intended that the embodiments and figures disclosed herein are to be considered illustrative rather than restrictive.

FIG. 1 (panels A-D) shows that BTN2A2-Fc enhances interaction and co-localization of CD45 with TCR signaling proteins. (A) Jurkat cells were stimulated for 3 min with immobilized anti-CD3 antibody (10 μg/ml) in the presence or absence of BTN2A2-Fc (10 μg/ml). Cells were lysed in IP buffer, immunoprecipitated with anti-CD45 antibody and immunoblotted for total Zap70 and CD3ζ. Right panel show intensity plots depicted as mean±SD (N=3) (B) Immunostaining analysis shows segregation of CD45 from CD3ζ in the presence or absence of recombinant BTN2A2-Fc protein (10 mg/ml) after Jurkat cells activation by plate-bound anti-CD3 antibody (10 mg/ml) for 3 min. Right panel shows quantification of CD3ζ and CD45 colocalization in the presence or absence of BTN2A2-Fc proteins from multiple fields. At least 50 cells from each group were included in the analysis shown in the right panel. (C) Co-immunoprecipitation experiments were performed using anti-CD3E antibody in Jurkat cells with the same condition as in (A), followed by immunoblot with anti-CD45 antibody. (D) CD45-specific phosphatase activity measured in immunoprecipitate from samples in panel C using FDP (Fluorescein Diphosphate, Tetraammonium Salt) substrate as described in the methods. Data depicted as mean±SD (N=3). One-way ANOVA with Tukey's multiple comparison test; p<0.05 (*), p<0.01 (**), p<0.001 (***).

FIG. 2 (panels A-H) shows that CD45 phosphatase co-immunoprecipitated with BTN2A2. (A) Jurkat cells were immunoprecipitated with anti-BTN2A2 antibody or IgG and blotted with CD45 antibody at unstimulated state or after activation anti-CD3 (1 mg/ml) for 48 hours. Input is ~3% of cell lysate. One representative experiment is depicted. (B) Jurkat cells were treated with both plate-bound anti-CD3 antibody (lg/ml) for 48 hours in the presence of recombinant BTN2A2-Fc or Fc-tag protein. Cells were lysed in IP buffer and immunoprecipitated with protein G and immunoblotting performed with anti-CD45 antibody. Input is ~3% of cell lysate. One representative experiment is depicted. (C) Jurkat cells were treated with anti-CD3 antibody (1 μg/ml) in the presence of recombinant BTN2A2-Fc or Fc-tag protein for 48 hours and crosslinked with BS3, lysed, immunoprecipitated with protein G and immunoblotted with anti-CD45 antibody. Input is ~3% of cell lysate. One representative blot is depicted. (D) Ribbon diagram representation of protein-protein interaction between BTN2A2 (pink) and CD45 (PTPRC)(white) showing potential amino acids (blue sticks) determining their interaction. Two residues N419 and N468, key glycosylation sites, located in the fibronectin domain of CD45 that is critical for the interaction is indicated in a spherical model. (E) Western blot demonstrating the expression of CD45 in wild type Jurkat cells (lane 1) and Jurkat cells with CD45 knock-down (CD45-KD) (lane 2); CD45 expression in CD45-KD cells in which wild type CD45 or mutant CD45 (double mutant—N419A and N468A) were expressed is depicted in lanes 3-4. GAPDH was used internal control. (F) Co-immunoprecipitation of CD45 with BTN2A2 as described in panel B in Jurkat cells lacking CD45 in which wild type CD45 or mutant CD45 (double mutant—N419A and N468) were expressed and activated (1 mg/ml of anti-CD3 antibody for 48 hrs) in the presence of recombinant BTN2A2-Fc or Fc. (G) Western blot demonstrating expression of the CD45 isoforms at baseline and at 48 hours after stimulation with anti-CD3 antibody in Jurkat cells. (H) Western blot analysis of CD45 isoforms (CD45RO and RA) following immunoprecipitation with protein G agarose beads of lysates obtained from activated Jurkat cells (1 mg/ml of anti-CD3 antibody for 48 hrs) in the presence of recombinant BTN2A2-Fc or Fc. One representative experiment is depicted.

FIG. 3 (panels A-D) shows that BTN2A2-Fc enhances Tregs and suppress Th17 cells differentiation in in-vitro mixed lymphocytes reaction (MLR) (A) Flow cytometry analysis plot of primary CD4+ T cells (from spleen and lymph nodes of Foxp3-GFP transgenic mice) incubated with bound 1 μg/ml anti-CD3 and in the presence or absence of 10 μg/ml BTN2A2-Fc fusion protein in MLR for 7 days as described in methods and analyzed for CD4+CD25+Foxp3−GFP+ve cells expression. As positive control, effects of TGF-b (1 ng/ml) under the same conditions are also depicted. Right panel show summary plot depicted as mean±SD for 3 independent experiments. One-way ANOVA with Tukey's multiple comparison test; p<0.05 (*). (B) Flow cytometry analysis of primary CD4+ T cells (isolated from murine spleen and lymph nodes) incubated for 5 days with anti-CD3 (0.5 mg/ml) antibody alone or anti-CD3+TGF-b (1.5 ng/ml) +IL6 (10 ng/ml) +IL-1β (10 ng/ml) and/or recombinant BTN2A2-Fc (10 μg/ml) in MLR and analyzed for CD4+RORgt+ve cells. Right panel show summary plot depicted as mean±SD for 3 independent experiments. One-way ANOVA with Tukey's multiple comparison test: p<0.05 (*). (C) Flow cytometry analysis of Foxp3-GFP+ve cell (%) population in total CD4+ T cells co-cultured with dendritic cells (DC) at day 7. Purified T cells were incubated in RPMI ex-vivo for day7 with DC cells (1:10 ratio) isolated from wild-type or BTN2A2−/− mice. (D) Flow cytometry analysis of Foxp3−GFP+ve cell (%) population in total CD4+ cells co-cultured with B cells at day 7. Purified T-cells were incubated ex-vivo for day7 with B-cell (1:5 ratio) isolated from wild-type or BTN2A2−/− mice. Data for experiments in (C-D) are depicted as mean±SD (n=3). Unpaired t-test; p<0.05 (*), p<0.001 (***).

FIG. 4 (panels A-C) shows that inhibition of CD45 phosphatase activity in primary immune cells blocks BTN2A2 mediated Treg differentiation and Th17 suppression. (A) Immunoblot analysis of phosphorylated ZAP-70 (p-Zap70) and total Zap70 in Jurkat cells pretreated with CD45 phosphatase inhibitor for 1 hr and stimulated for 3 min with plate-bound anti-CD3 (10 μg/ml) antibody in the presence or absence of recombinant BTN2A2-Fc (10 μg/ml). (B) Flow cytometry analysis plot of primary CD4+ T cells (from spleen and lymph nodes of Foxp3−GFP transgenic mice) incubated with bound 1 μg/ml anti-CD3 and/or 10 μg/ml BTN2A2-Fc fusion protein in MLR for 7 days in absence or presence of CD45 phosphatase inhibitor (125 nM) and analyzed for CD4+CD25+Foxp3−GFP+ve cells expression. Right panel shows summary plot depicted as mean±SD for 3 independent experiments. (C) Flow cytometry analysis of primary CD4+ T cells (isolated from murine spleen and lymph nodes) incubated for 5 days with anti-CD3 (0.5 mg/ml) antibody alone or anti-CD3+TGF-b (1.5 ng/ml) +IL6 (10 ng/ml) +IL-1β (10 ng/ml) and/or recombinant BTN2A2-Fc (10 μg/ml) in MLR for 7 days in absence or presence of CD45 phosphatase inhibitor (125 nM) and analyzed for CD4+RORgt+ve cells. Right panel shows summary plot depicted as mean±SD. One-way ANOVA test with Tukey's multiple comparison test; p<0.01 (**), p<0.001 (***).

FIG. 5 (panels A-1) shows that BTN2A2-Fc ameliorates crescentic glomerulonephritis in mice induced by nephrotoxic serum (NTS). (A) Schematic protocol of NTS-induced nephrotoxic glomerulonephritis model and histological changes. GBM=glomerular basement membrane. (B) Quantification of mean±SEM proteinuria (on Day 7) in mice with NTS induced glomerulonephritis treated with recombinant BTN2A2-Fc protein or vehicle control (control) (N=12/group); Mann-Whitney p<0.001(***). (C-D) Representative images of glomerular injury by PAS staining. Crescent formation in mice with NTS induced glomerulonephritis treated with treated with recombinant BTN2A2-Fc protein or control (D). Scale bars are 25 mm. Summary panel (C) depicts quantification of % glomeruli with crescents (mean±SEM, N=12/group); Mann-Whitney p<0.001(***). (E-F) Relative Foxp3 (D) and RORgt (E) expression compared to GAPDH as internal control in CD4+ T cells purified from spleen and lymph nodes from mice with NTS induced glomerulonephritis treated with BTN2A2-Fc protein or control. Data depicted as mean+SEM (N=8/group). Mann-Whitney test; p<0.05 (*). (G) Quantitative PCR analysis show CD5 expression in CD4+ T cells from mice with NTS induced glomerulonephritis treated with BTN2A2-Fc protein or control. N=8/group. (H-I) Immunoblot analysis of IL17A in kidney tissue lysate of mice with glomerulonephritis treated with recombinant BTN2A2-Fc or control (H). Panel I shows quantification of western blot. Data represented as mean f SEM. N=4 per group. Unpaired t-test; p<0.05 (*).

FIG. 6 (panels A-H) depicts BTN2A2 (−/−) mice showing exacerbated crescentic glomerulonephritis. (A) Scatter plot shows quantification of mean±SEM of proteinuria in wild-type and BTN2A2 (−/−) mice with nephrotoxic glomerulonephritis on Day 7 (N=15). Mann-Whitney test; p<0.01 (**). (B, C) Representative images of glomerular injury by PAS staining. Wild-type mice show minimal glomerular injury with a low dose of nephrotoxic serum, while BTN2A2 (−/−) mice show severe glomerulonephritis (B). Scale bars are 25 mm. Summary data are depicted as mean±SEM of % Glomerular crescents (C). N=15 per group for all experiments. Mann-Whitney test; p<0.001 (***). (D-E) Relative Foxp3 mRNA (D) and RORgt mRNA (E) compared to GAPDH as internal control in CD4+ve T cells purified from spleen and lymph nodes cells from wild-type and BTN2A2-knockout mice with nephrotoxic serum induced glomerulonephritis. (F) Quantitative PCR analysis show CD5 expression in CD4+ T cells from wild-type and BTN2A2-knockout mice with nephrotoxic serum induced glomerulonephritis. N=7/group. (G-H) Immunoblotting for IL17A expression in in kidney tissue lysate using anti-IL-17A antibody (G) and quantitation (H) from wild-type and BTN2A2 null mice with nephrotoxic serum induced glomerulonephritis. n>4 per group. Unpaired t-test; p<0.01 (**), p<0.001 (***).

FIG. 7 (panels A-H) shows that BTN2A2-Fc rescues fetus resorption and increases litter size in CBA/J×DBA/2 pregnancy model. (A-B). Pregnant CBA/J×DBA/2 mice treated with BTN2A2 throughout pregnancy has improved litter size (A), reduced resorption (B) compared to untreated mice. As additional control. DBA/2×CBA/J mice has been included. Data (A, B) represented as mean±SEM. N=11 per group for all experiments. Kruskal-Wallis test with Dunn's test for multiple comparison: p<0.05 (*), p<0.01 (**), p<0.001 (***). (C-D) Quantitative PCR (C) and flowcytometry (D) analysis show increase in Foxp3 expressing CD4+ T cells in pregnant CBA/J×DBA/2J mice treated with recombinant BTN2A2-Fc as compared to control mice. N=8/group (E) Flow cytometry analysis (left panel) show reduced number of CD4+RORgt+ve cells in pregnant CBA/J×DBA/2J mice treated with recombinant BTN2A2-Fc as compared to control mice. Right panel shows quantification of the summary data. N=8/group. (F) Quantitative PCR analysis show CD5 expression in CD4+ T cells in pregnant CBA/J×DBA/2J mice and treated with recombinant BTN2A2-Fc as compared to control mice. N=8/group. (G, H) Immunoblot analysis of IL17A in placental tissue lysate of pregnant CBA/J×DBA/2J mice treated with recombinant BTN2A2-Fc or control (left panel). Right panel shows quantification of summary data. N=3 per group. One-way ANOVA test with Tukey's multiple comparison test for experiments in C-G; p<0.05 (*), p<0.01 (**), p<0.001 (***).

FIG. 8 (panels A-D) shows that BTN2A2-Fc enhances Tregs and suppress Th17 cells differentiation in in vitro human PBMC mixed lymphocyte reactions. (A-B). Flow cytometry plots showing percentage of Treg cells (CD4+CD25+Foxp3+ve cells) in human PBMC incubated with/without recombinant BTN2A2-Fc (10 mg/ml) in MLR for 7 days in absence or presence of CD45 phosphatase inhibitor (125 nM). Data depicted as mean+/−SD showing percentage of Treg cells (CD4+CD25+Foxp3+ve cells) among total CD4+ cells. N=4 per group for all experiments. Unpaired 2-tailed t test; p<0.001 (***). (C-D). Flow cytometry plots showing percentage of CD4+RORgt+ve (Th17 cells) in human PBMC incubated for 3 days with anti-CD3 antibody (0.5 mg/ml) only or anti-CD3 antibody+TGF-b (1.5 ng/ml) +IL-6 (10 ng/ml) +IL-1b (10 ng/ml) in absence or presence of recombinant BTN2A2-Fc (10 mg/ml) in MLR for 7 days in absence or presence of CD45 phosphatase inhibitor (125 nM). Data represent mean standard deviation. N=3 per group for all experiments. One-way ANOVA test with Tukey's multiple comparison test; p<0.01 (**).

FIG. 9 shows a representative schematic of the model by which BTN2A2 as a co-inhibitor of TCR signaling via CD45 phosphatase.

FIG. 10 (panels A-D) shows that BTN2A2-Fc regulates T-cell receptor signaling. (A) Schematic diagram showing the recombinant human BTN2A2-Fc fusion. The recombinant fusion gene consisting of the two extracellular domains (IgV and IgC2) of BTN2A2 molecule (green color) cloned along with the Fc region of a human IgG1 antibody (orange color). The chimeric protein (BTN2A2-Fe) is separated by IEGRMD (SEQ ID NO:57) spacer polypeptide. (B) Coomassie blue staining of purified recombinant BTN2A2-Fc protein on reducing (SDS) gel with a protein ladder in the left column and a band corresponding to BTN2A2-Fc (~55 kDa) in the right two columns. Lower panel shows western blot of recombinant BTN2A2-Fc using specific anti-human BTN2A2 antibody. (C) IL-2 secretion from Jurkat cells incubated for 24 hrs with plate-immobilized anti-CD3 antibody (1 μg/ml) in the absence or presence of immobilized recombinant BTN2A2-Fc (10 μg/ml), 293-cell derived human BTN2A2-Fc (R&D systems) or mouse BTN2A2 (R&D systems), and immobilized immunoglobulin Fc domain as negative control is depicted (N=3 or 4). (D) Immunoblot analysis of phosphorylated proteins. p-Zap70 and p-CD3-ζ and total Zap70 and CD3-ζ in Jurkat cells stimulated for 3 min with immobilized anti-CD3 antibody (10 μg/ml) in the presence of immobilized recombinant BTN2A2-Fc (10 μg/ml) or immunoglobulin Fc domain (10 μg/ml). Right panel shows relative intensity plots as mean±SD (N=3). One-way ANOVA with Tukey's multiple comparison test; p<0.05 (*), p<0.01 (**), p<0.001 (***).

FIG. 11 depicts pervanadate studies. Jurkat cells treated with permanent phosphatase inhibitor (Pervanadate) plus minus BTN2A2-Fc or left untreated. After cell lysis, phosphorylated protein, p-Zap70 and p-CD3-ζ were analyzed by Western blot. Total Zap70 and CD3-ζ were controls.

FIG. 12 depicts Jurkat cells stimulated with/without anti-CD3 antibody (1 μg/ml) for 48 hrs and the lysates were immunoblotted with anti-CD45 or anti-BTN2A2 antibody. GAPDH expression was used as a loading control.

FIG. 13 (panels A-B) depicts CRISPR-Cas9 deletion of CD45 and site directed mutagenesis of CD45. (A) a representative schematic of CD45 sequences showing location of gRNA sequences complementarity. Lower panel show electropherogram of the sequence deleted upstream of exon-1 to downstream of exon-3. nucleotide sequence: SEQ ID NO:46: amino acid sequence: SEQ ID NO:47. (B) Electropherogram showing change of nucleotides, AAT to GCT at Asn419Ala and AAT to GCT at Asn468Ala in CD45 mutants gene. Top left: nucleotide sequence-SEQ ID NO:48. amino acid sequence-SEQ ID NO:49; Top right: nucleotide sequence-SEQ ID NO:50, amino acid sequence-SEQ ID NO:51; Bottom left: nucleotide sequence-SEQ ID NO:52, amino acid sequence-SEQ ID NO:53; Bottom Right: nucleotide sequence-SEQ ID NO:54, amino acid sequence-SEQ ID NO:55.

FIG. 14 (panels A-C) shows that BTN2A2-Fc inhibits CD4+ve T-cells proliferation, apoptosis and IL2 secretion. (A) Flow cytometry plots of CellTrace violet stained purified mouse CD4+ve cells incubated with immobilized anti-CD3 and anti-CD28 antibody (0.5 μg/ml) in the presence of immobilized recombinant BTN2A2-Fc (10 μg/ml) or immunoglobulin Fc domain (10 μg/ml) for Day 3. Right panels shows bar graph of un-proliferated cells (GO) percentage. (B) Flow cytometry plots of Annexin-V stained purified mouse CD4+ve cells incubated with immobilized anti-CD3 and anti-CD28 antibody (0.5 μg/ml) in the presence of immobilized recombinant BTN2A2-Fc (10 μg/ml) or immunoglobulin Fc domain (10 μg/ml) for Day 3. Right panels shows bar graph of apoptotic cells percentage. (C) Flow cytometry plots of IL2 positive purified mouse CD4+ve cells incubated with immobilized anti-CD3 and anti-CD28 antibody (0.5 μg/ml) in the presence of immobilized recombinant BTN2A2-Fc (10 μg/ml) or immunoglobulin Fc domain (10 μg/ml) for Day 3. Right panels shows bar graph of IL2 positive cells percentage. Data are depicted as mean±SD (n=4). Unpaired t-test; p<0.001 (***).

FIG. 15 (panels A-F) show qPCR studies for candidate molecules in T cell differentiation pathway. Quantitative PCR analysis for IL-2RB (A), Foxp3 (B). IL-21 (C), TBX21 (D), GATA3 (E) and Smad-3 (F) in primary CD4 cells incubated for 1 Day and 5 days in absence or presence of anti-CD3 antibody and/or recombinant BTN2A2-Fc. All data represented as mean±standard deviation. n=3 per group for all experiments. One-way ANOVA with Tukey's multiple comparisons test; p<0.05 (*), p<0.01 (**), p<0.001 (***).

FIG. 16 (panels A-B) shows BTN2A2 null mice generation/characterization. (A) Btn2a2−/− mouse m by CRISPR/Cas-mediated genome engineering (cyagen.com). The Btn2a2 gene (NCBI Reference Sequence: NM_175938; Ensembl: ENSMUSG00000053216) is located on mouse chromosome 13. Eight exons are identified, with the ATG start codon in exon-2 and the TAG stop codon in exon-8 (Transcript Btn2a2-203: ENSMUST00000110433). Exons 2~8 (covers 100.0% of the coding region) were selected as target site. Cas9 and gRNA co-injected into fertilized eggs for KO mouse production. The pups were genotyped by PCR followed by sequencing analysis. The size of effective KO region: ~10500 bp. gRNA target sequences SEQ ID NOs: gRNA1: SEQ ID NO:39; gRNA2: SEQ ID NO:40: gRNA3: SEQ ID NO:41; gRNA4: SEQ ID NO:42. Genotyping primer SEQ ID NOs: F1: SEQ ID NO:43; F2: SEQ ID NO:44; RI: SEQ ID NO:45. (B) Agarose gel electrophoresis of genotyping PCR. Homozygotes (Homo) mouse shows one band with 700 bp, heterozygotes (Het) mouse with two bands at 700 bp and 500 bp; wildtype mouse (WT) one band at 500 bp.

FIG. 17 (panels A-B) shows BTN2A2 expression in antigen presenting cells. qPCR analysis of BTN2A2 expression in B cells (A) and Dendritic cells (B) populations purified using magnetic beads from spleen and lymph nodes of wildtype and BTN2A2-KO mice. Left panels shows flow cytometry plots of purified cells, and right panels shows fold change in RNA expression of BTN2A2 transcript compared to GAPDH.

FIG. 18 contains a schematic protocol of abortion prone model.

FIG. 19 depicts a schematic showing binding of BTN2A2 with Myelin oligodendrocyte glycoprotein (MOG). The protein structure prediction software (Prime, www.schrodinger.com) was used to evaluate the binding of BTN2A2 to MOG, the antigen for multiple sclerosis and other MOG-related autoimmune diseases. Root mean square of atomic positions (RMSD) was <1A.

### DESCRIPTION OF THE INVENTION

All references cited herein are incorporated by reference in their entirety as though fully set forth. Unless defined otherwise, technical and scientific terms used herein have the same meaning as commonly understood by one of ordinary skill in the art to which this invention belongs. One skilled in the art will recognize many methods and materials similar or equivalent to those described herein, which could be used in the practice of the present invention. Indeed, the present invention is in no way limited to the methods and materials described. For purposes of the present invention, the following terms are defined below.

As used herein the term “about” when used in connection with a referenced numeric indication means the referenced numeric indication plus or minus up to 5% of that referenced numeric indication, unless otherwise specifically provided for herein. For example, the language “about 50%” covers the range of 45% to 55%. In various embodiments, the term “about” when used in connection with a referenced numeric indication can mean the referenced numeric indication plus or minus up to 4%, 3%, 2%, 1%. 0.5%, or 0.25% of that referenced numeric indication, if specifically provided for in the claims.

Percent (%) sequence identity with respect to a reference polypeptide sequence is the percentage of amino acid residues in a candidate sequence that are identical with the amino acid residues in the reference polypeptide sequence, after aligning the sequences and introducing gaps, if necessary, to achieve the maximum percent sequence identity, and not considering any conservative substitutions as part of the sequence identity. Alignment for purposes of determining percent amino acid sequence identity can be achieved in various ways that are known for instance, using publicly available computer software such as BLAST, BLAST-2, ALIGN or Megalign (DNASTAR) software. Appropriate parameters for aligning sequences are able to be determined, including algorithms needed to achieve maximal alignment over the full length of the sequences being compared.

“Linked” as used herein in the context of linked peptides, polypeptides, or proteins refers to being “connected to” either directly or indirectly. Indirect linkage can be mediated by a polypeptide linker such as poly-glycine or a glycine-serine polypeptide, for example, (GGGGS)n (SEQ ID NO:6)n or (GGGGGS)n (SEQ ID NO:7)n, wherein n is an integer. In various embodiments, n is an integer from 1-10, 10-20, 20-30, 30-40, or 50-100. Other such linkers are known in the art and is considered to be encompassed by this term.

Described herein we demonstrate that BTN2A2 functions as a ligand for CD45, binding both to the TCR complex and CD45RO isoform on T cell surfaces, resulting in retention of CD45 phosphatase activity proximal to the TCR complex. Consequently, the enhanced phosphatase activity reduces downstream TCR signaling, which in turn promotes regulatory T cell (Treg) expansion, suppressing T effector cell differentiation. We further demonstrate the significant immunomodulatory effects of BTN2A2 in a mouse model of autoimmune glomerulonephritis, showing that BTN2A2 genetic deficiency exacerbated kidney injury, whereas administration of recombinant BTN2A2 limited disease severity. Furthermore, in a second mouse model of immune-mediated spontaneous abortion, treatment of pregnant dams with recombinant BTN2A2 helped animals tolerate their pregnancies reducing fetal loss.

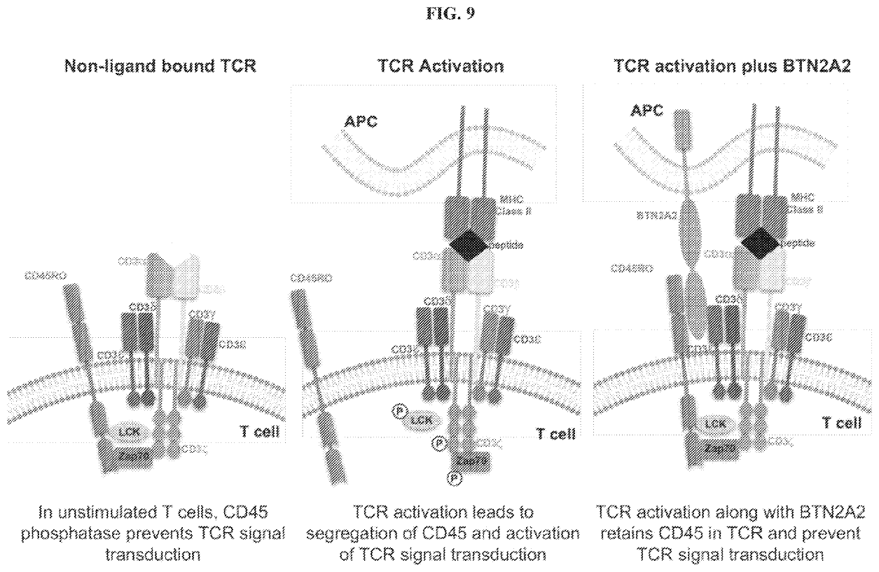

Data included herein provide multiple lines of evidence showing that BTN2A2 functions as a ligand for CD45, preferentially binding to CD45RO isoform, and that it functions to prevent segregation of CD45 from the TCR/CD3 complex, resulting in sustained CD45's phosphatase activity within the immune synapse which reduces downstream signaling cascade (See FIG. 9) Consequently, BTN2A2 reduces proliferation of effector Th17 cells while promoting differentiation of T regulatory cells, and reduces severity of NTS-induced glomerulonephritis and immune mediated pregnancy loss in mouse models of immunologic diseases. Our findings suggest that during cognate T cell/APC interactions, APC-produce BTN2A2 which ligates T cell-expressed CD45RO to the TCR, functioning as a molecular brake at the time of activation of the TCR complex by peptide/MHC complexes. Our data suggest that in CD45RO+ T cells, BTN2A2 binds to both CD45 and CD3e, likely maintaining them in proximity. enabling CD45 phosphatase activity to persist, enabling dephosphorylation of CD3 ITAM domains and Zap70 and limiting downstream TCR signals required for fill T cell activation.

Our data also indicate that BTN2A2 enhances CD45's phosphatase activity within the TCR complex which modulates the TCR signaling but does not fully prevent it, and consequently, permits expansion of Tregs while reducing effector T cell differentiation/expansion. In prior studies, transient targeting of CD45 induced potent antigen-specific regulatory T cells and induced transplant tolerance. Our data suggests that BTN2A2 expressed on APCs and DCs is likely the endogenous ligand for CD45 on T cells. CD45 is expressed as multiple spliced products and it was believed that CD45RA and CD45RB are replaced by CD45RO following T cell activation. These studies suggest that BTN2A2 predominantly binds to the CD45RO isoform that is expressed on activated T cells, signifying specificity for BTN2A2's actions. While multiple human glycoproteins, including galectin-1 and CD22, were shown by others to bind CD45, there is no clear evidence that these putative ligands modulate CD45 phosphatase activity. Other identified ligands for CD45 include pUL11, a viral protein expressed by cytomegalovirus-infected cells, as well as E3/49K protein expressed by adenovirus-infected cells; both of these viral proteins may play roles in inhibiting anti-viral immunity by inhibiting TCR signaling, although precise mechanisms remain unclear.

We show that BTN2A2-Fc limits clinical manifestations of glomerular crescent formation and proteinuria in murine NTS glomerulonephritis and reduces miscarriages in a distinct orthogonal mouse model, both of which were associated with augmented Treg and reduced Th17 cell populations. CD5, a T cell activation marker was attenuated by BTN2A2-Fc. Our data further indicate that BTN2A2-Fc is a potentially useful immune modulator with translational potential in a variety of immune-mediated diseases and transplantation. Our observations are an important step forward in understanding how naturally occurring proteins can modify inflammatory events by interacting with CD45 to sustain TCR complex phosphatase activity, increasing Treg and decreasing Th17 cell populations.

Since CD45 is also expressed on B cells, it is possible that some of the beneficial effects of BTN2A2 in our animal studies may be derived from its actions on B cells.

Our findings suggest that BTN2A2 ligation of CD45 phosphatase to the TCR complex leads to dampened TCR signaling resulting in expansion of Tregs and suppression of Th17 cells. While we demonstrate a beneficial effect of BTN2A2 in two orthogonal models of autoimmunity/immune tolerance, our data suggest that BTN2A2/CD45 signaling pathway could be targeted in a wide variety of immune-mediated diseases such as inflammatory bowel disease, rheumatoid arthritis, multiple sclerosis, and transplant rejections.

Accordingly, various embodiments of the present invention are based, at least in part, on these finding.

Various embodiments provide for a method of reducing CD3 dependent T cell signaling in a subject in need thereof, comprising administering butyrophilin A2 (BTN2A2), a BTN2A2 fragment thereof, a BTN2A2-related isoform, or a BTN2A2-related isoform fragment, to the subject.

Various embodiments provide for a method of increasing T-regulatory (Treg) cells, or decreasing T-helper 17 (Th17) cells, or both in a subject in need thereof, comprising: administering butyrophilin A2 (BTN2A2), a BTN2A2 fragment thereof, a BTN2A2-related isoform, or a BTN2A2-related isoform fragment, to the subject.

Based on the phylogenetic tree, BTN1A1, B2N2A1, BTN2A2, BTN3A1, BTN3A2 and BTN3A3 have similar structure, particularly their extracellular domains, and are closely related. It is therefore expected that all of these isoforms, particularly, their extracellular domains, to have similar function to BTN2A2 in its ability to regulate the balance of Treg and Th17 cells, and be used in accordance with various embodiments of the invention.

In various embodiments, the subject is pregnant, and the method inhibits or reduces the likelihood of miscarriage of a fetus by increasing Treg cells.

In various embodiments, the subject has renal damage.

In various embodiments, the subject is an organ, tissue or cell therapy transplant recipient. In various embodiments, the method reduces the likelihood of organ, tissue or cell transplant rejection. In various embodiments, the method reduces a need for an immunosuppressive therapy or reduces the amount of immunosuppressive therapy administered to the subject. In various embodiments, the method reduces the toxic effects of immunosuppressive therapy on the subject.

In various embodiments, the subject has an autoimmune or inflammatory disorder. In various embodiments, the autoimmune disorder or inflammatory disorder comprises allergy, asthma, glomerulonephritis, inflammatory bowel disease or Crohn's disease, rheumatoid arthritis, an autoimmune neurological disease, antibody mediated transplant rejection, infantile cholestasis, haemophagocytic lymphohistiocytosis, erythrocytic haemophagocytosis, malnutrition, systemic lupus erythematosus (lupus), psoriasis, sarcoidosis, myasthenia gravis or HIV. In various embodiments, the autoimmune neurological disease is multiple sclerosis. In various embodiments, the autoimmune neurological disease is myelin oligodendrocyte glycoprotein (MOG) antibody disease or Alzheimer's disease.

In various embodiments, the butyrophilin A2 (BTN2A2) or the BTN2A2 fragment thereof is fused to a Fc domain or a fragment of the Fc domain (“BTN2A2-Fc fusion protein”) and/or another half-life extending moiety such as PEG, albumin. lipidation, cholesterol, or glycosylation. In various embodiments, the BTN2A2-related isoform or the BTN2A2-related isoform fragment is fused to the Fc domain or the fragment of the Fc domain (“BTN2A2-related isoform-Fe fusion protein”), typically that of a human IgG such as human IgG1, IgG2, IgG3 or IgG4, which optionally may be modified to increase or decrease a particular Fc-associated effector function.

In various embodiments, the butyrophilin A2 (BTN2A2) or the BTN2A2 fragment thereof is linked to a Fc domain or a fragment of the Fc domain (“BTN2A2-Fc fusion protein”) typically that of a human IgG such as human IgG1, IgG2, IgG3 or IgG4, which optionally may be modified to increase or decrease a particular Fe-associated effector function. In various embodiments, the BTN2A2-related isoform or the BTN2A2-related isoform fragment is linked to the Fc domain or the fragment of the Fc domain (“BTN2A2-related isoform-Fc fusion protein”) typically that of a human IgG such as human IgG1, IgG2, IgG3 or IgG4, which optionally may be modified to increase or decrease a particular Fc-associated effector function.

In various embodiments, the linker is G, polyserine, polyglycine, glycine-serine, GGGGSn (SEQ ID NO:6)n, GGGGGSn (SEQ ID NO:7)n, leucine zipper, r aliphatic, or helical peptides.

In various embodiments, a spacer peptide is between the BTN2A2, BTN2A2 fragment, the BTN2A2-related isoform, or the BTN2A2-related isoform fragment, and the Fc domain or the fragment of the Fc domain. That is, there is a spacer between the BTN2A2 portion and the Fc domain portion. For example, the fusion protein comprises BTN2A2-spacer-Fc domain, or BTN2A2-spacer-fragment of the Fc domain, or BTN2A2 fragment-spacer-Fc domain, or BTN2A2 fragment-spacer-fragment of the Fc domain. These examples are not to limit the possible combinations, but rather to provide clarity.

Examples of spacers include but are not limited to IEGRMDDISSTMVRS (SEQ ID NO:56). IEGRMD (SEQ ID NO:57). EAEAEAK (SEQ ID NO:58), SIINFEKL (SEQ ID NO:59), and GGGS (SEQ ID NO: 60).

In various embodiments, the Fc domain comprises mutations selected from Met 208Leu, Asn214Ser or both, in reference to SEQ ID NO:4. The mutation is capable of extending the half-life of B2N2A2Fc. (See e.g., Saunders, “Conceptual Approaches to Modulating Antibody Effector Functions and Circulation Half-Life”, Front Immunol. 2019; 10: 1296)

In various embodiments, the Fc domain has the amino acid sequence of SEQ ID NO:4 or SEQ ID NO:5.

In various embodiments, the BTN2A2 fragment comprises the amino acid sequence of SEQ ID NO:3.

In various embodiments, a fusion protein having SEQ ID NO:1 or SEQ ID NO:2 is administered in accordance with the embodiments described herein. In various embodiments, a fusion protein having SEQ ID NO: 1 or SEQ ID NO:2 wherein the spacer IEGRMDDISSTMVRS (SEQ ID NO:56) is replaced with IEGRMD (SEQ ID NO: 57), is administered in accordance with the embodiments described herein.

In various embodiments, the butyrophilin A2 (BTN2A2) or the BTN2A2 fragment thereof, the BTN2A2-related isoform or the BTN2A2-related isoform fragment is modified by glycosylation or PEGylation. These modifications are capable of extending the half-life of the butyrophilin A2 (BTN2A2) or the BTN2A2 fragment thereof, the BTN2A2-related isoform or the BTN2A2-related isoform fragment.

Modification by PEGylation can extend the half-life of the butyrophilin A2 (BTN2A2) or the BTN2A2 fragment thereof, the BTN2A2-related isoform or the BTN2A2-related isoform fragment. Examples of PEGylation include Veronese and Mero, “The Impact of PEGylation on Biological Therapies”, Biodrugs 2008; 22 (5): 315-329, incorporated herein by reference as though fully set forth. Thus, for example PEG or branched PEG (PEG₂) can be used. Exemplary sizes of PEG or PEG₂ include but are not limited 1-5 kDa, 5 kDa, 5-10 kDa, 10-15 kDa, 15-20 kDa, 20-25 kDa, 25-30 kDa, 30-40 kDa, 40-50 kDa, 50 kDa, 50-60 kDa, 60-75 kDa.

Another approach to improve the pharmacokinetic (PK) properties of BTN2A2 or a fragment thereof is its attachment to albumin, e.g., human serum albumin (HSA), which precludes proteolytic degradation and rapid renal filtration (due to the relatively large size of HSA of approximately 66 kDa). Still another approach to improve the pharmacokinetic (PK) properties of BTN2A2 or a fragment thereof is glycosylation. Yet another approach to improve the pharmacokinetic (PK) properties of BTN2A2 or a fragment thereof is its attachment to lipids or to cholesterol.

In various embodiments, the BTN2A2 fragment comprises an extracellular domain of BTN2A2. In various embodiments, the BTN2A2 fragment does not comprise a transmembrane domain. In various embodiments, the BTN2A2 fragment does not comprise a cytoplasmic domain. In various embodiments the BTNA2 fragment binds to CD45, e.g., CD45RO.

In various embodiments, the BTN2A2-related isoform is BTN1A1, B2N2A1, BTN3A1, BTN3A2 or BTN3A3 or a fragment or fusion polypeptide comprising any of the foregoing, optionally an Fe fusion polypeptide typically that of a human IgG such as human IgG1, IgG2, IgG3 or IgG4, which optionally may be modified to increase or decrease a particular Fc-associated effector function.

In various embodiments, the BTN2A2-related isoform fragment does not comprise the transmembrane domain, the cytoplasmic domain, or both. In various embodiments the BTN2A2-related isoform fragment binds to CD45, e.g., CD45RO.

Various embodiments of the invention provide for a method of treating a disease or condition in a subject in need thereof, comprising: administering butyrophilin A2 (BTN2A2), a BTN2A2 fragment thereof, a BTN2A2-related isoform, or a BTN2A2-related isoform fragment or a fusion polypeptide comprising any of the foregoing, optionally an Fc fusion polypeptide, to the subject.

In various embodiments, the disease or condition is an autoimmune disorder or disorder associated with inflammation.

In various embodiments, the autoimmune or inflammatory disorder is glomerulonephritis, inflammatory bowel disease, rheumatoid arthritis, an autoimmune neurological disease, antibody mediated transplant rejection, infantile cholestasis, haemophagocytic lymphohistiocytosis, erythrocytic hemophagocytes, malnutrition, systemic lupus erythematosus, myasthenia gravis or HIV.

In various embodiments, the autoimmune neurological disease is multiple sclerosis. In various embodiments, the autoimmune neurological disease is myelin oligodendrocyte glycoprotein (MOG) antibody disease or Alzheimer's disease.

In various embodiments, the disease or condition is renal damage.

In various embodiments, the disease or condition is organ, tissue or cell transplant rejection and the method reduces the likelihood of the organ, tissue or cell transplant rejection. In various embodiments, the disease or condition is organ, tissue or cell transplant rejection and the method reduces a need for an immunosuppressive therapy or reducing an amount of immunosuppressive therapy needed by the subject. In various embodiments, the treatment results in a reduction or elimination of the otherwise toxic effects of the immunosuppressive therapy in the subject.

In various embodiments, the butyrophilin A2 (BTN2A2) or the BTN2A2 fragment thereof is fused to a Fc domain or a fragment of the Fc domain (“BTN2A2-Fc fusion protein”) typically that of a human IgG such as human IgG1, IgG2. IgG3 or IgG4, which optionally may be modified to increase or decrease a particular Fc-associated effector function. In various embodiments, the BTN2A2-related isoform or the BTN2A2-related isoform fragment is fused to the Fc domain or the fragment of the Fc domain (“BTN2A2-related isoform-Fc fusion protein”) typically that of a human IgG such as human IgG1, IgG2, IgG3 or IgG4, which optionally may be modified to increase or decrease a particular Fc-associated effector function.

In various embodiments, the butyrophilin A2 (BTN2A2) or the BTN2A2 fragment thereof is linked to a Fc domain or a fragment of the Fc domain (“BTN2A2-Fc fusion protein”) typically that of a human IgG such as human IgG1, IgG2, IgG3 or IgG4, which optionally may be modified to increase or decrease a particular Fe-associated effector function. In various embodiments, the BTN2A2-related isoform or the BTN2A2-related isoform fragment is linked to the Fc domain or the fragment of the Fc domain (“BTN2A2-related isoform-Fc fusion protein”). In various embodiments, the linker is G, polyserine, polyglycine, glycine-serine, GGGGSn (SEQ ID NO:6)n. GGGGGSn (SEQ ID NO:7)n, leucine zipper, r aliphatic, or helical peptides.

In various embodiments, a spacer peptide, optionally from 2-50 amino acids, is between the BTN2A2, BTN2A2 fragment, the BTN2A2-related isoform, or the BTN2A2-related isoform fragment, and the Fc domain or the fragment of the Fc domain, typically that of a human IgG such as human IgG1, IgG2, IgG3 or IgG4, which optionally may be modified to increase or decrease a particular Fc-associated effector function. That is, there is a spacer between the BTN2A2 portion and the Fc domain portion. For example, the fusion protein comprises BTN2A2-spacer-Fc domain, or BTN2A2-spacer-fragment of the Fc domain, or BTN2A2 fragment-spacer-Fc domain, or BTN2A2 fragment-spacer-fragment of the Fc domain. These examples are not to limit the possible combinations, but rather to provide clarity.

Examples of spacers include but are not limited to IEGRMDDISSTMVRS (SEQ ID NO:56), IEGRMD (SEQ ID NO:57). EAEAEAK (SEQ ID NO:58), SIINFEKL (SEQ ID NO:59), and GGGS (SEQ ID NO: 60).

In various embodiments, the Fc domain, e.g., an IgG1 Fc domain, comprises mutations selected from Met 208Leu, Asn214Ser or both, in reference to SEQ ID NO:4. The mutation is capable of extending the half-life of B2N2A2Fc. (See e.g., Saunders, “Conceptual Approaches to Modulating Antibody Effector Functions and Circulation Half-Life”, Front Immunol. 2019; 10: 1296)

In various embodiments, the Fc domain has the amino acid sequence of SEQ ID NO:4 or SEQ ID NO:5.

In various embodiments, a fusion protein having SEQ ID NO:1 or SEQ ID NO:2 is administered in accordance with the embodiments described herein. In various embodiments, a fusion protein having SEQ ID NO:1 or SEQ ID NO:2 wherein the spacer IEGRMDDISSTMVRS (SEQ ID NO:56) is replaced with IEGRMD (SEQ ID NO: 57), is administered in accordance with the embodiments described herein.

In various embodiments, the butyrophilin A2 (BTN2A2) or the BTN2A2 fragment thereof, the BTN2A2-related isoform or the BTN2A2-related isoform fragment is modified by attachment to an albumin, e.g., human serum albumin (HAS).

In various embodiments, the butyrophilin A2 (BTN2A2) or the BTN2A2 fragment thereof, the BTN2A2-related isoform or the BTN2A2-related isoform fragment is modified by glycosylation or PEGylation. These modifications are capable of extending the half-life of the butyrophilin A2 (BTN2A2) or the BTN2A2 fragment thereof, the BTN2A2-related isoform or the BTN2A2-related isoform fragment.

Modification by PEGylation can extend the half-life of the butyrophilin A2 (BTN2A2) or the BTN2A2 fragment thereof, the BTN2A2-related isoform or the BTN2A2-related isoform fragment. Examples of PEGylation include Veronese and Mero, “The Impact of PEGylation on Biological Therapies”, Biodrugs 2008; 22 (5): 315-329, incorporated herein by reference as though fully set forth. Thus, for example PEG or branched PEG (PEG₂) can be used. Exemplary sizes of PEG or PEG₂ include but are not limited 1-5 kDa, 5 kDa, 5-10 kDa, 10-15 kDa, 15-20 kDa, 20-25 kDa, 25-30 kDa, 30-40 kDa, 40-50 kDa, 50 kDa, 50-60 kDa, 60-75 kDa.

In various embodiments, the BTN2A2 fragment comprises an extracellular domain of BTN2A2. In various embodiments, the BTN2A2 fragment does not comprise a transmembrane domain. In various embodiments, the BTN2A2 fragment does not comprise a cytoplasmic domain. In various embodiments the BTN2A2 fragment binds to CD45, e.g., CD45RO.

In various embodiments, the BTN2A2-related isoform is BTN1A1, B2N2A1, BTN3A1, BTN3A2 or BTN3A3. In various embodiments, the BTN2A2-related isoform fragment does not comprise the transmembrane domain, the cytoplasmic domain, or both.

Various embodiments of the invention provide for a fusion protein, comprising: a butyrophilin A2 (BTN2A2), a BTN2A2 fragment, a BTN2A2-related isoform, or a BTN2A2-related isoform fragment; and an Fc domain or a fragment of the Fc domain, typically that of a human IgG such as human IgG1, IgG2, IgG3 or IgG4, which optionally may be modified to increase or decrease a particular Fe-associated effector function.

In various embodiments, the fusion protein is not a fusion protein having SEQ ID NO: 1. In various embodiments, the fusion protein is not a fusion protein having SEQ ID NO:1, wherein the spacer IEGRMDDISSTMVRS (SEQ ID NO:56) is replaced with IEGRMD (SEQ ID NO:57).

In various embodiments, the Fc domain or the fragment of the Fc domain is from an IgG1 antibody.

In various embodiments, the Fc domain or the fragment of the Fc domain, typically that of a human IgG such as human IgG1, IgG2, IgG3 or IgG4, which optionally may be modified to increase or decrease a particular Fc-associated effector function and may comprise one or more mutations. In various embodiments, the mutation is Met208Leu. Asn214Ser, or both, in reference to SEQ ID NO:4. Mutations to the Fc domain provide for extended half-life of the fusion protein.

In various embodiments, the Fc domain has the amino acid sequence of SEQ ID NO:4 or SEQ ID NO:5. Alternatively, the BTN2A2 may comprise an Fc variant selected from any of the following:

### Alternative Fc Variants

As mentioned the subject BTN2A2 polypeptides and fragments may comprise an Fc region which optionally may be mutagenized to modulate an Fe-associated effector function. In some embodiments, one or more amino acid modifications are introduced into the Fc region, thereby generating an Fc region variant or Fc variant. An Fc region may comprise a C-terminal region of an immunoglobulin heavy chain that comprises a hinge region, CH2 domain, CH3 domain, or any combination thereof. As used herein, an Fc region includes native sequence Fc regions and variant Fc regions. The Fc region variant may comprise a human Fc region sequence (e.g., a human IgG1, IgG2. IgG3 or IgG4 Fc region) comprising an amino acid modification (e.g., a substitution, addition, or deletion) at one or more amino acid positions.

In some embodiments, a variant Fc region comprises at least one amino acid modification in the Fc region. Combining amino acid modifications are also useful. For example, the variant Fc region may include two, three, four, five, etc. substitutions therein, e.g. of the specific Fc region positions identified herein.

In some embodiments, the altered effector function is reduced effector function. In some embodiments, the altered effector function is increased effector function Effector functions generally refer to a biological event resulting from the interaction of an antibody Fc region with an Fc receptor or ligand. Non-limiting effector functions include Clq binding, complement dependent cytotoxicity (CDC), Fe receptor binding, antibody-dependent cell-mediated cytotoxicity (ADCC), antibody-dependent cellular phagocytosis (ADCP), cytokine secretion, immune complex-mediated antigen uptake by antigen presenting cells, down regulation of cell surface receptors (e.g. B cell receptor), and B cell activation. In some cases, antibody-dependent cell-mediated cytotoxicity (ADCC) refers to a cell-mediated reaction in which nonspecific cytotoxic cells expressing Fc receptors (e.g., natural killer cells, neutrophils, macrophages) recognize bound antibody on a target cell, subsequently causing lysis of the target cell. In some cases, complement dependent cytotoxicity (CDC) refers to lysing of a target cells in the presence of complement, where the complement action pathway is initiated by the binding of Clq to antibody bound with the target.

In certain cases, it is beneficial to reduce the effector function. In some instances, modifications in the Fc region generate an Fc variant with (a) decreased antibody-dependent cell-mediated cytotoxicity ADCC), (b) decreased complement mediated cytotoxicity (CDC), and/or (c) decreased affinity for Cl q. In some embodiments, the Fc region is modified to decrease antibody dependent cellular cytotoxicity (ADCC), decreased antibody-dependent cell-mediated phagocytosis (ADCP), decreased complement mediated cytotoxicity (CDC), and/or decreased affinity for Clq by modifying one or more amino acids at the following positions: 234, 235, 236, 238, 239, 240, 241, 243, 244, 245, 247, 248, 249, 252, 254, 255, 256, 258, 262, 263, 264, 265, 267, 268, 269, 270, 272, 276, 278, 280, 283, 285, 286, 289, 290, 292, 293, 294, 295, 296, 298, 299, 301, 303, 305, 307, 309, 312, 313, 315, 320, 322, 324, 325, 326, 327, 329, 330, 331, 332, 333, 334, 335, 337, 338, 340, 360, 373, 376, 378, 382, 388, 389, 398, 414, 416, 419, 430, 433, 434, 435, 436, 437, 438 or 439 (EU numbering). In some embodiments, the variant Fc region is selected from Table 3.

TABLE 3

| Exemplary Variant Fc Regions | Exemplary Effect on Effector |
|---|---|
| and Mutation(s) | Function |
| E233P | Decreases binding to FcγRI, II, |
| | III |
| S228P, L235E SPLE IN IgG4 | Decreases binding to FcγRI |
| L235E | Decreases binding to FcγRs |
| L234A, L235A | Decreases binding to FcγRI, II |
| | and III |
| L234A, L235A, G237A | Decreases binding to FcγRI, II |
| | and III, C1q |
| L234A, L235A, P329G | Decreases binding to FcγRI, II |
| | and III, C1q |
| L234F, L235E, P331S | Decreases binding to FcγRI, II |
| | and III, C1q |
| L2345A, L235E, G237A | Decreases binding to FcγRI, II |
| | and III, C1q |
| L234A, L235E, G237A, P331S | Decreases binding to FcγRI, II |
| | and III, C1q |
| L234A, L235E, G237A, A330S, | Decreases binding to FcγRI, II |
| P331S | and III, and C1q |
| L234A, L235A, G237A, P238S, | Decreases binding to FcγRI, IIa, |
| H268A, A330S, P331S (IGG1Σ) | IIb, and IIIa |
| L234A, L235A, P329A | Decreases binding to FcγRI, II, |
| | IIb, III, and C1q |
| G236R, L328R | Decreases binding to FcγRI, II |
| | and III |
| G237A | Decreases binding to FcγRII |
| F241A | Decreases binding to C1q |
| V264A | Decreases binding to C1q |
| D265A | Decreased binding to FcγRI, II, |
| | IIIc |
| D265A, N297A | Decreased binding to FcγRI, II, |
| | III, and C1q |
| D265A, N297G | Decreased binding to FcγRI, II, |
| | III, and C1q |
| D270A | Reduced C1q binding |
| N297A, G, D, Q | Elimination of n-linked |
| | glycosylation, decreased binding |
| | to FcγRI, II, III, and C1q |
| P329A, G, R | Reduced C1q binding |
| A330L | Reduced C1q binding |
| P331A or P331S | Reduced C1q binding |
| S228P | Prevent IgG4 Fab arm exchange |
| S228P, F234A, L235A, (IgG4) | Decreased binding to FcγRI, IIa, |
| | IIIa |
| H268Q, V309L, A330S, P331S | Decreased binding to FcγRI, II, |
| (IgG2M4) | III, and C1q |
| V234A, G237A, P238S, H268A, | Decreased binding to FcγRI, IIa, |
| V309L, A330S, P331S (IGG2Σ) | IIb, IIIa, and C1q |
| K322A | Decreased binding to C1q |
| N297A, OR N297Q OR N297G | Decreased binding to FcγR and |
| | C1q binding |
| H268Q, V309L, A330S, P331S | Decreased binding to FcγR and |
| | C1q |
| V234A, G237A, P238S, H268A, | Decreased binding to FcγR and |
| V309L, A330S, P331S | C1q |
| High Mannose Glycosylation | Decreases binding to C1q |

In vitro assays to assess ADCC activity of a molecule of interest are well known in the art. Non-limiting examples of in vitro assays to assess ADCC activity of a molecule of interest is described in U.S. Pat. Nos. 5,500,362 and 5,821,337. Useful effector cells for use in such assays include peripheral blood mononuclear cells (PBMC), monocytes, macrophages, and Natural Killer (NK) cells.

In some embodiments, variant Fc regions exhibit reduced effector function as compared with wild-type human IgG1. Non-limiting examples of Fc mutations in IgG1 that, in certain instances, reduce ADCC and/or CDC include substitutions at one or more of positions: 231, 232, 234, 235, 236, 237, 238, 239, 264, 265, 267, 269, 270, 297, 299, 318, 320, 322, 325, 327, 328, 329, 330, and 331 in IgG1, where the numbering system of the constant region is that of the EU index as set forth by Kabat.

In some embodiments, the variant Fc region comprises an IgG1 Fc region comprising an N297A substitution, according to the EU numbering system. In some embodiments, the variant Fc region comprises an IgG1 Fc region comprising an N297Q substitution, according to the EU numbering system. In some embodiments, the variant Fc region comprises an IgG1 Fc region comprising an N297D substitution, according to the EU numbering system. In some embodiments, the variant Fc region comprises an IgG1 Fc region comprising an D265 A substitution, according to the EU numbering system. In some embodiments, the variant Fc region comprises an IgG1 Fc region comprising an S228P substitution, according to the EU numbering system. In some embodiments, the variant Fc region comprises an IgG1 Fc region comprising an L235 A substitution, according to the EU numbering system. In some embodiments, the variant Fc region comprises an IgG1 Fc region comprising an L237A substitution, according to the EU numbering system. In some embodiments, the variant Fc region comprises an IgG1 Fc region comprising an L234A substitution, according to the EU numbering system. In some embodiments, the variant Fc region comprises an IgG1 Fc region comprising an E233P substitution, according to the EU numbering system. In some embodiments, the variant Fc region comprises an IgG1 Fc region comprising an L234 V substitution, according to the EU numbering system. In some embodiments, the variant Fc region comprises an IgG1 Fc region comprising an C236 deletion, according to the Kabat numbering system. In some embodiments, the variant Fc region comprises an IgG1 Fc region comprising a P238 A substitution, according to the EU numbering system. In some embodiments, the variant Fc region comprises an IgG1 Fc region comprising an A327Q substitution, according to the EU numbering system. In some embodiments, the variant Fc region comprises an IgG1 Fc region comprising a P329 A substitution, according to the EU numbering system. In some embodiments, the variant Fc region comprises an IgG1 Fc region comprising an P329G substitution, according to the EU numbering system. In some embodiments, the variant Fc region comprises an IgG1 Fc region comprising an L235E substitution, according to the EU numbering system. In some embodiments, the variant Fc region comprises an IgG1 Fc region comprising an P331S substitution, according to the EU numbering system. In some embodiments, the variant Fc region comprises an IgG1 Fc region comprising an L234F substitution, according to the EU numbering system. In some embodiments, the variant Fc region comprises an IgG1 Fc region comprising a 235G substitution, according to the EU numbering system. In some embodiments, the variant Fc region comprises an IgG1 Fc region comprising a 235 Q substitution, according to the EU numbering system. In some embodiments, the variant Fc region comprises an IgG1 Fe region comprising an 235R substitution, according to the EU numbering system. In some embodiments, the variant Fc region comprises an IgG1 Fc region comprising an 235S substitution, according to the EU numbering system. In some embodiments, the variant Fc region comprises an IgG1 Fc region comprising an 236F substitution, according to the EU numbering system. In some embodiments, the variant Fc region comprises an IgG1 Fc region comprising an 236R substitution, according to the EU numbering system. In some embodiments, the variant Fc region comprises an IgG1 Fc region comprising an 237E substitution, according to the EU numbering system. In some embodiments, the variant Fc region comprises an IgG1 Fc region comprising an 237K substitution, according to the EU numbering system. In some embodiments, the variant Fc region comprises an IgG1 Fc region comprising an 237N substitution, according to the EU numbering system. In some embodiments, the variant Fc region comprises an IgG1 Fc region comprising an 237R substitution, according to the EU numbering system. In some embodiments, the variant Fc region comprises an IgG1 Fc region comprising a 238 A substitution, according to the EU numbering system. In some embodiments, the variant Fc region comprises an IgG1 Fc region comprising an 238E substitution, according to the EU numbering system. In some embodiments, the variant Fc region comprises an IgG1 Fe region comprising an 238G substitution, according to the EU numbering system. In some embodiments, the variant Fc region comprises an IgG1 Fc region comprising an 238H substitution, according to the EU numbering system. In some embodiments, the variant Fc region comprises an IgG1 Fc region comprising a 2381 substitution, according to the EU numbering system. In some embodiments, the variant Fc region comprises an IgG1 Fc region comprising an 238V substitution, according to the EU numbering system. In some embodiments, the variant Fc region comprises an IgG1 Fc region comprising an 238W substitution, according to the EU numbering system. In some embodiments, the variant Fc region comprises an IgG1 Fc region comprising an 238Y substitution, according to the EU numbering system. In some embodiments, the variant Fc region comprises an IgG1 Fc region comprising a 248 A substitution, according to the EU numbering system. In some embodiments, the variant Fc region comprises an IgG1 Fc region comprising an 254D substitution, according to the EU numbering system. In some embodiments, the variant Fc region comprises an IgG1 Fc region comprising an 254E substitution, according to the EU numbering system. In some embodiments, the variant Fc region comprises an IgG1 Fc region comprising an 254G substitution, according to the EU numbering system. In some embodiments, the variant Fc region comprises an IgG1 Fe region comprising an 254H substitution, according to the EU numbering system. In some embodiments, the variant Fc region comprises an IgG1 Fc region comprising a 254I substitution, according to the EU numbering system. In some embodiments, the variant Fc region comprises an IgG1 Fc region comprising an 254N substitution, according to the EU numbering system. In some embodiments, the variant Fc region comprises an IgG1 Fc region comprising an 254P substitution, according to the EU numbering system. In some embodiments, the variant Fc region comprises an IgG1 Fc region comprising an 254Q substitution, according to the EU numbering system. In some embodiments, the variant Fc region comprises an IgG1 Fe region comprising an 254T substitution, according to the EU numbering system. In some embodiments, the variant Fc region comprises an IgG1 Fc region comprising an 254V substitution, according to the EU numbering system. In some embodiments, the variant Fc region comprises an IgG1 Fc region comprising an 255N substitution, according to the EU numbering system. In some embodiments, the variant Fc region comprises an IgG1 Fc region comprising an 256H substitution, according to the EU numbering system. In some embodiments, the variant Fc region comprises an IgG1 Fc region comprising an 256K substitution, according to the EU numbering system. In some embodiments, the variant Fc region comprises an IgG1 Fc region comprising an 256R substitution, according to the EU numbering system. In some embodiments, the variant Fc region comprises an IgG1 Fc region comprising an 256V substitution, according to the EU numbering system. In some embodiments, the variant Fc region comprises an IgG1 Fc region comprising an 264S substitution, according to the EU numbering system. In some embodiments, the variant Fc region comprises an IgG1 Fc region comprising an 265H substitution, according to the EU numbering system. In some embodiments, the variant Fc region comprises an IgG1 Fc region comprising an 265K substitution, according to the EU numbering system. In some embodiments, the variant Fc region comprises an IgG1 Fc region comprising a 265 S substitution, according to the EU numbering system. In some embodiments, the variant Fc region comprises an IgG1 Fc region comprising a 265 Y substitution, according to the EU numbering system. In some embodiments, the variant Fc region comprises an IgG1 Fc region comprising an 267G substitution, according to the EU numbering system. In some embodiments, the variant Fc region comprises an IgG1 Fc region comprising an 267H substitution, according to the EU numbering system. In some embodiments, the variant Fc region comprises an IgG1 Fc region comprising a 2671 substitution, according to the EU numbering system. In some embodiments, the variant Fc region comprises an IgG1 Fc region comprising an 267K substitution, according to the EU numbering system. In some embodiments, the variant Fc region comprises an IgG1 Fc region comprising an 268K substitution, according to the EU numbering system. In some embodiments, the variant Fc region comprises an IgG1 Fc region comprising an 269N substitution, according to the EU numbering system. In some embodiments, the variant Fc region comprises an IgG1 Fc region comprising an 269Q substitution, according to the EU numbering system. In some embodiments, the variant Fc region comprises an IgG1 Fc region comprising an 270A substitution, according to the EU numbering system. In some embodiments, the variant Fc region comprises an IgG1 Fe region comprising an 270G substitution, according to the EU numbering system. In some embodiments, the variant Fc region comprises an IgG1 Fc region comprising an 270M substitution, according to the EU numbering system. In some embodiments, the variant Fc region comprises an IgG1 Fc region comprising an 270N substitution, according to the EU numbering system. In some embodiments, the variant Fc region comprises an IgG1 Fc region comprising an 271T substitution, according to the EU numbering system. In some embodiments, the variant Fc region comprises an IgG1 Fc region comprising an 272N substitution, according to the EU numbering system. In some embodiments, the variant Fc region comprises an IgG1 Fc region comprising an 279F substitution, according to the EU numbering system. In some embodiments, the variant Fc region comprises an IgG1 Fc region comprising an 279K substitution, according to the EU numbering system. In some embodiments, the variant Fc region comprises an IgG1 Fc region comprising an 279L substitution, according to the EU numbering system. In some embodiments, the variant Fc region comprises an IgG1 Fc region comprising an 292E substitution, according to the EU numbering system. In some embodiments, the variant Fc region comprises an IgG1 Fc region comprising an 292F substitution, according to the EU numbering system. In some embodiments, the variant Fc region comprises an IgG1 Fc region comprising an 292G substitution, according to the EU numbering system. In some embodiments, the variant Fc region comprises an IgG1 Fc region comprising an 292L substitution, according to the EU numbering system. In some embodiments, the variant Fc region comprises an IgG1 Fc region comprising an 293S substitution, according to the EU numbering system. In some embodiments, the variant Fc region comprises an IgG1 Fc region comprising an 301W substitution, according to the EU numbering system. In some embodiments, the variant Fc region comprises an IgG1 Fc region comprising an 304E substitution, according to the EU numbering system. In some embodiments, the variant Fc region comprises an IgG1 Fe region comprising an 31IE substitution, according to the EU numbering system. In some embodiments, the variant Fc region comprises an IgG1 Fc region comprising a 311 G substitution, according to the EU numbering system. In some embodiments, the variant Fc region comprises an IgG1 Fc region comprising an 311S substitution, according to the EU numbering system. In some embodiments, the variant Fc region comprises an IgG1 Fc region comprising an 316F substitution, according to the EU numbering system. In some embodiments, the variant Fc region comprises an IgG1 Fc region comprising an 327T substitution, according to the EU numbering system. In some embodiments, the variant Fc region comprises an IgG1 Fc region comprising an 328V substitution, according to the EU numbering system. In some embodiments, the variant Fc region comprises an IgG1 Fc region comprising a 329 Y substitution, according to the EU numbering system. In some embodiments, the variant Fc region comprises an IgG1 Fc region comprising an 330R substitution, according to the EU numbering system. In some embodiments, the variant Fc region comprises an IgG1 Fc region comprising an 339E substitution, according to the EU numbering system. In some embodiments, the variant Fc region comprises an IgG1 Fc region comprising an 339L substitution, according to the EU numbering system. In some embodiments, the variant Fc region comprises an IgG1 Fc region comprising a 3431 substitution, according to the EU numbering system. In some embodiments, the variant Fc region comprises an IgG1 Fc region comprising a 343 V substitution, according to the EU numbering system. In some embodiments, the variant Fc region comprises an IgG1 Fc region comprising an 373A substitution, according to the EU numbering system. In some embodiments, the variant Fc region comprises an IgG1 Fc region comprising an 373G substitution, according to the EU numbering system. In some embodiments, the variant Fc region comprises an IgG1 Fc region comprising a 373 S substitution, according to the EU numbering system. In some embodiments, the variant Fc region comprises an IgG1 Fe region comprising an 376E substitution, according to the EU numbering system. In some embodiments, the variant Fc region comprises an IgG1 Fc region comprising an 376W substitution, according to the EU numbering system. In some embodiments, the variant Fc region comprises an IgG1 Fc region comprising an 376Y substitution, according to the EU numbering system. In some embodiments, the variant Fc region comprises an IgG1 Fc region comprising an 380D substitution, according to the EU numbering system. In some embodiments, the variant Fc region comprises an IgG1 Fc region comprising an 382D substitution, according to the EU numbering system. In some embodiments, the variant Fc region comprises an IgG1 Fc region comprising an 382P substitution, according to the EU numbering system. In some embodiments, the variant Fc region comprises an IgG1 Fc region comprising an 385P substitution, according to the EU numbering system. In some embodiments, the variant Fc region comprises an IgG1 Fc region comprising an 424H substitution, according to the EU numbering system. In some embodiments, the variant Fc region comprises an IgG1 Fc region comprising an 424M substitution, according to the EU numbering system. In some embodiments, the variant Fc region comprises an IgG1 Fc region comprising an 424V substitution, according to the EU numbering system. In some embodiments, the variant Fc region comprises an IgG1 Fe region comprising an 434L substitution, according to the EU numbering system. In some embodiments, the variant Fc region comprises an IgG1 Fc region comprising an 438G substitution, according to the EU numbering system. In some embodiments, the variant Fc region comprises an IgG1 Fc region comprising an 439E substitution, according to the EU numbering system. In some embodiments, the variant Fc region comprises an IgG1 Fc region comprising an 439H substitution, according to the EU numbering system. In some embodiments, the variant Fc region comprises an IgG1 Fc region comprising an 439Q substitution, according to the EU numbering system. In some embodiments, the variant Fc region comprises an IgG1 Fc region comprising an 440A substitution, according to the EU numbering system. In some embodiments, the variant Fc region comprises an IgG1 Fc region comprising an 440D substitution, according to the EU numbering system. In some embodiments, the variant Fc region comprises an IgG1 Fc region comprising an 440E substitution, according to the EU numbering system. In some embodiments, the variant Fc region comprises an IgG1 Fc region comprising an 440F substitution, according to the EU numbering system. In some embodiments, the variant Fc region comprises an IgG1 Fc region comprising an 440M substitution, according to the EU numbering system. In some embodiments, the variant Fc region comprises an IgG1 Fc region comprising an 440T Fc region substitution, according to the EU numbering system. In some embodiments, the variant Fc region comprises an IgG1 Fc region comprising an 440V substitution, according to the EU numbering system.

In some embodiments, the variant Fc region comprises an IgG1 Fc region L234A, L235E, G237A, A330S, and/or P331 S by EU numbering. In some embodiments, the variant Fc region comprises an IgG1 Fc region comprising E233P, according to the EU numbering system. In some embodiments, the variant Fc region comprises an IgG4 Fc region comprising S228P and L235E. In some embodiments, the variant Fc region comprises an IgG1 Fc region comprising L235E, according to the EU numbering system. In some embodiments, the variant Fc region comprises an IgG1 Fc region comprising L234A and L235A, according to the EU numbering system. In some embodiments, the variant Fc region comprises an IgG1 Fe region comprising L234A, L235A, and G237A, according to the EU numbering system. In some embodiments, the variant Fc region comprises an IgG1 Fc region comprising L234 A, L235A, P329G, according to the EU numbering system. In some embodiments, the variant Fc region comprises an IgG1 Fc region comprising L234F, L235E, and P331S, according to the EU numbering system. In some embodiments, the variant Fc region comprises an IgG1 Fc region comprising L234A, L235E, and G237A, according to the EU numbering system. In some embodiments, the variant Fc region comprises an IgG1 Fe region comprising L234 A, L235E, G237A, and P331S, according to the EU numbering system. In some embodiments, the variant Fc region comprises an IgG1 Fc region comprising L234 A, L235A, G237A, P238S, H268A, A330S, and P331S (IgG1), according to the EU numbering system. In some embodiments, the variant Fc region comprises an IgG1 Fc region comprising L234A, L235A, and P329A, according to the EU numbering system. In some embodiments, the variant Fc region comprises an IgG1 Fc region comprising G236R and L328R, according to the EU numbering system. In some embodiments, the variant Fc region comprises an IgG1 Fc region comprising G237A, according to the EU numbering system. In some embodiments, the variant Fc region comprises an IgG1 Fc region comprising F241 A, according to the EU numbering system. In some embodiments, the variant Fc region comprises an IgG1 Fc region comprising V264A, according to the EU numbering system. In some embodiments, the variant Fc region comprises an IgG1 Fc region comprising D265A, according to the EU numbering system. In some embodiments, the variant Fc region comprises an IgG1 Fc region comprising D265A and N297A, according to the EU numbering system. In some embodiments, the variant Fc region comprises an IgG1 Fc region comprising D265A and N297G, according to the EU numbering system. In some embodiments, the variant Fc region comprises an IgG1 Fc region comprising D270A, according to the EU numbering system. In some embodiments, the variant Fc region comprises an IgG1 Fc region comprising N297A, according to the EU numbering system. In some embodiments, the variant Fc region comprises an IgG1 Fc region comprising N297G, according to the EU numbering system. In some embodiments, the variant Fc region comprises an IgG1 Fc region comprising N297D, according to the EU numbering system. In some embodiments, the variant Fc region comprises an IgG1 Fc region comprising N297Q, according to the EU numbering system. In some embodiments, the variant Fc region comprises an IgG1 Fc region comprising P329A, according to the EU numbering system. In some embodiments, the variant Fc region comprises an IgG1 Fc region comprising P329G, according to the EU numbering system. In some embodiments, the variant Fc region comprises an IgG1 Fc region comprising P329R, according to the EU numbering system. In some embodiments, the variant Fc region comprises an IgG1 Fc region comprising A330L, according to the EU numbering system. In some embodiments, the variant Fc region comprises an IgG1 Fc region comprising P331 A, according to the EU numbering system. In some embodiments, the variant Fc region comprises an IgG1 Fc region comprising P331S, according to the EU numbering system. In some embodiments, the variant Fc region comprises an IgG2 Fc region. In some embodiments, the variant Fc region comprises an IgG4 Fc region. In some embodiments, the variant Fc region comprises an IgG4 Fe region comprising S228P, according to the EU numbering system. In some embodiments, the variant Fc region comprises an IgG4 Fc region comprising S228P, F234A, and L235A, according to the EU numbering system. In some embodiments, the variant Fc region comprises an IgG2-IgG4 cross-subclass (IgG2/G4) Fc region. In some embodiments, the variant Fc region comprises an IgG2-IgG3 cross-subclass Fc region. In some embodiments, the variant Fc region comprises an IgG2 Fc region comprising H268Q. V309L, A330S, and P33 IS, according to the EU numbering system. In some embodiments, the variant Fc region comprises an IgG2 Fc region comprising V234A, G237A, P238S, H268A. V309L, A330S, and P33 IS, according to the EU numbering system. In some embodiments, an antibody comprises a Fc region comprising high mannose glycosylation.

In some embodiments, the one or more mutations relative to a wildtype Fc region comprises or consists of L234A, L235A, and P329G by EU numbering. In some embodiments, the one or more mutations relative to a wildtype Fc region comprises or consists of L234 A, L235E, G237A. A330S, and P33IS by EU numbering. In some embodiments, the one or more mutations relative to a wildtype Fc region is selected from the group consisting of: N297A/Q/G: L235A/G237A/E318A; L234A/L235A; G236R/L328R; S298G/T299A: L234F/L235E/P331 S; H268Q/V309L/A330S/P331S; L234A/L235A/P329G; V234A/G237A/P238S/H268A/V309L/A330S/P331 S; and L234F/L235E/D265A. In some embodiments, the variant Fc region comprises an IgG4 Fc region comprising a S228P substitution, according to the EU numbering system. In some embodiments, the variant Fc region comprises an IgG4 Fc region comprising an A330S substitution, according to the EU numbering system. In some embodiments, the variant Fc region comprises an IgG4 Fc region comprising a P331S substitution, according to the EU numbering system.

In some embodiments, the variant Fc region comprises an IgG2 Fc region comprising an A330S substitution, according to the EU numbering system. In some embodiments, the variant Fc region comprises an IgG2 Fc region comprising an P331S substitution, according to the EU numbering system. In some embodiments, the variant Fc region comprises an IgG2 Fc region comprising an 234A substitution, according to the EU numbering system. In some embodiments, the variant Fc region comprises an IgG2 Fc region comprising an 237A substitution, according to the EU numbering system.

In some embodiments, the variant Fc region comprises IgG1 Fc region, and wherein the one or more mutations comprises (a) 297A, 297Q, 297G, or 297D, (b) 279F, 279K, or 279L, (c) 228P, (d) 235 A, 235E, 235G, 235Q, 235R, or 235S, (e) 237A, 237E, 237K, 237N, or 237R, (f) 234 A, 234V, or 234F, (g) 233P, (h) 328 A, (i) 327Q or 327T, (j) 329 A, 329G, 329Y, or 329R (k) 331 S, (l) 236F or 236R, (m) 238 A, 238E, 238G, 238H, 238L, 238V, 238 W, or 238Y, (n) 248 A. (o) 254D, 254E, 254G, 254H, 254I, 254N, 254P, 254Q, 254T, or 254V, (p) 255N, (q) 256H, 256K, 256R, or 256V, (r) 264S, (s) 265H, 265K, 265S, 265Y, or 265A, (t) 267G, 267H, 267L, or 267K, (u) 268K, (v) 269N or 269Q, (w) 270 A, 270G, 270M, or 270N, (x) 271T, (y) 272N, (z) 292E, 292F, 292G, or 292L, (aa) 293S, (bb) 301W, (cc) 304E, (dd) 311E, 311G, or 311S, (ee) 316F, (ff) 328V, (gg) 330R, (hh) 339E or 339L, (ii) 343L or 343V, (jj) 373 A, 373G, or 373 S, (kk) 376E, 376W, or 376Y, (ll) 380D, (mm) 382D or 382P, (nn) 385P, (oo) 424H, 424M, or 424V, (pp) 4341, (qq) 438G, (rr) 439E. 439H, or 439Q, (ss) 440A, 440D, 440E, 440F, 440M, 440T, or 440V, (tt) K322A, (uu) L235E, (vv) L234A and L235A, (ww) L234A, L235A, and G237A, (xx) L234A, L235A. and P329G, (yy) L234F, L235E, and P331S. (zz) L234A, L235E, and G237A, (aaa), L234A, L235E, G237A, and P33 IS (bbb) L234A, L235A, G237A, P238S, H268A, A330S, and P331S, (ccc) L234A, L235A, and P329A, (ddd) G236R and L328R, (eee) G237A, (fff) F241A, (ggg) V264A, (hhh) D265A, (iii) D265A and N297A, (jjj) D265A and N297G, (kkk) D270A, (lll) A330L, (mmm) P331A or P331S, or (nnn) E233P, (ooo) L234A, L235E, G237A, A330S, and P331S or (ppp) any combination of (a)-(uu), per EU numbering.

In various embodiments, the fusion protein further comprising a spacer peptide, optionally from 2-50 amino acids, between the BTN2A2, BTN2A2 fragment, the BTN2A2-related isoform, or the BTN2A2-related isoform fragment, and the Fc domain or the fragment of the Fc domain. That is, there is a spacer between the BTN2A2 portion and the Fc domain portion. For example, the fusion protein comprises BTN2A2-spacer-Fc domain, or BTN2A2-spacer-fragment of the Fc domain, or BTN2A2 fragment-spacer-Fc domain, or BTN2A2 fragment-spacer-fragment of the Fc domain. These examples are not to limit the possible combinations, but rather to provide clarity.

Examples of spacers include but are not limited to IEGRMDDISSTMVRS (SEQ ID NO:56), IEGRMD (SEQ ID NO:57). EAEAEAK (SEQ ID NO:58), SIINFEKL (SEQ ID NO:59), and GGGS (SEQ ID NO: 60).

In various embodiments, the BTN2A2 fragment comprises the extracellular domain of BTN2A2. In various embodiments, the BTN2A2 fragment does not comprise the transmembrane domain. In various embodiments, the BTN2A2 fragment does not comprise the cytoplasmic domain. In various embodiments the BTN2A2 fragment binds to CD45, e.g., CD45RO.

In various embodiments, the BTN2A2-related isoform is BTN1A1, B2N2A1. BTN3A1, BTN3A2 or BTN3A3. In various embodiments, the BTN2A2-related isoform fragment does not comprise the transmembrane domain, the cytoplasmic domain, or both.

In various embodiments, the fusion protein has the sequence of SEQ ID NO:2.

Various embodiments provide for a modified polypeptide, comprising: a butyrophilin A2 (BTN2A2), a BTN2A2 fragment, a BTN2A2-related isoform, or a BTN2A2-related isoform fragment or a combination thereof, wherein the butyrophilin A2 (BTN2A2), the BTN2A2 fragment, the BTN2A2-related isoform, or the BTN2A2-related isoform fragment, or the combination thereof is PEGylated, lipidated or glycosylated.

These modifications are capable of extending the half-life of the butyrophilin A2 (BTN2A2) or the BTN2A2 fragment thereof, the BTN2A2-related isoform or the BTN2A2-related isoform fragment.

Modification by PEGylation can extend the half-life of the butyrophilin A2 (BTN2A2) or the BTN2A2 fragment thereof, the BTN2A2-related isoform or the BTN2A2-related isoform fragment. Examples of PEGylation include Veronese and Mero, “The Impact of PEGylation on Biological Therapies”, Biodrugs 2008; 22 (5): 315-329, incorporated herein by reference as though fully set forth. Thus, for example PEG or branched PEG (PEG₂) can be used. Exemplary sizes of PEG or PEG₂ include but are not limited 1-5 kDa, 5 kDa, 5-10 kDa, 10-15 kDa, 15-20 kDa, 20-25 kDa, 25-30 kDa, 30-40 kDa, 40-50 kDa, 50 kDa, 50-60 kDa, 60-75 kDa.

In various embodiments, the BTN2A2 fragment comprises the extracellular domain of BTN2A2. In various embodiments, the BTN2A2 fragment does not comprise the transmembrane domain, the cytoplasmic domain, or both. In various embodiments the BTN2A2 fragment binds to CD45, e.g. CD45RO,

In various embodiments, the BTN2A2-related isoform is BTN1A1, B2N2A1, BTN3A1, BTN3A2 or BTN3A3. In various embodiments, the BTN2A2-related isoform fragment does not comprise the transmembrane domain, the cytoplasmic domain, or both.

Various embodiments provide for an immunoassay to detect butyrophilin A2 (BTN2A2), a BTN2A2 fragment, a BTN2A2-related isoform, or a BTN2A2-related isoform fragment, or a combination thereof.

In various embodiments, the immunoassay is an ELISA.

In various embodiments, the immunoassay comprises an anti-butyrophilin A2 (BTN2A2) antibody, an anti-BTN2A2 fragment antibody, an anti-BTN2A2-related isoform antibody, or an anti-BTN2A2-related isoform fragment antibody, or a combination thereof immobilized on a solid support; and the solid support.

In various embodiments, the immunoassay further comprises a biological sample obtained from a subject. The subject may be one who desires information regarding levels butyrophilin A2 (BTN2A2), the BTN2A2 fragment, the BTN2A2-related isoform, or the BTN2A2-related isoform fragment.

Various embodiments, provide for a method of using an immunoassay of the present invention for patient monitoring, the method comprises: contacting a biological sample obtained from a patient to an immunoassay of the present invention; and detecting the level of butyrophilin A2 (BTN2A2), BTN2A2 fragments, BTN2A2-related isoforms, or BTN2A2-related isoform fragments, or a combination thereof.

In various embodiments, the method further comprises selecting or administering a treatment of the present invention as described herein. In various embodiments, the method further comprises selecting or administering one of the following treatments
(i) butyrophilin A2 (BTN2A2), a BTN2A2 fragment thereof, a BTN2A2-related isoform, or a BTN2A2-related isoform fragment;
(ii) a fusion polypeptide, comprising:
   a butyrophilin A2 (BTN2A2), a BTN2A2 fragment, a BTN2A2-related isoform, or a BTN2A2-related isoform fragment or a combination thereof; and an albumin, e.g., HSA, or an Fc domain or a fragment of an Fc domain, e.g., that of a human IgG such as human IgG1, IgG2, IgG3 or IgG4, which optionally may be modified to increase or decrease a particular Fe-associated effector function;
(iii) a modified polypeptide, comprising:
   a butyrophilin A2 (BTN2A2), a BTN2A2 fragment, a BTN2A2-related isoform, or a BTN2A2-related isoform fragment or a combination thereof, wherein the butyrophilin A2 (BTN2A2), the BTN2A2 fragment, the BTN2A2-related isoform, or the BTN2A2-related isoform fragment, or the combination thereof is PEGylated, lipidated or glycosylated; or
(iv) a non-BTN2A2 related treatment.

Various embodiments provide for a method of using an immunoassay of the present invention for patient stratification, the method comprising: contacting a biological sample obtained from a patient to an immunoassay of the present invention; detecting the level of butyrophilin A2 (BTN2A2), a BTN2A2 fragment, a BTN2A2-related isoform, or a BTN2A2-related isoform fragment, or a combination thereof; and stratifying the patient for treatment with a treatment of the present invention as described herein.

Various embodiments provide for a method of using an immunoassay of the present invention for patient stratification, the method comprising: contacting a biological sample obtained from a patient to an immunoassay of the present invention; detecting the level of butyrophilin A2 (BTN2A2), a BTN2A2 fragment, a BTN2A2-related isoform, or a BTN2A2-related isoform fragment, or a combination thereof; and stratifying the patient for treatment with one of the following treatments:
(i) butyrophilin A2 (BTN2A2), a BTN2A2 fragment thereof, a BTN2A2-related isoform, or a BTN2A2-related isoform fragment;
(ii) a fusion polypeptide, comprising:
   a butyrophilin A2 (BTN2A2), a BTN2A2 fragment, a BTN2A2-related isoform, or a BTN2A2-related isoform fragment or a combination thereof; and an Fc domain or a fragment of the Fc domain, typically that of a human IgG such as human IgG1, IgG2, IgG3 or IgG4, which optionally may be modified to increase or decrease a particular Fc-associated effector function;
(iii) a modified polypeptide, comprising:
   a butyrophilin A2 (BTN2A2), a BTN2A2 fragment, a BTN2A2-related isoform, or a BTN2A2-related isoform fragment or a combination thereof, wherein the butyrophilin A2 (BTN2A2), the BTN2A2 fragment, the BTN2A2-related isoform, or the BTN2A2-related isoform fragment, or the combination thereof is PEGylated, lipidated or glycosylated; or
(iv) a non-BTN2A2 related treatment.

Wild type Amino acid sequence
(SEQ ID NO: 1)
(237aa BTN2A2 EC domain (bold and italicized) +
15aa Xa and vector cloning seq + 227aa Fc region
(bold))
MEPAAALHFSLPASLLLLLLLLLLSLCALVSAQFTVVGPANPILAMVG
ENTTLRCHLSPEKNAEDMEVRWFRSQFSPAVFVYKGGRERTEEQMEEY
RGRITFVSKDINRGSVALVIHNVTAQENGIYRCYFQEGRSYDEAILRL
VVAGLGSKPLIEIKAQEDGSIWLECISGGWYPEPLTVWRDPYGEVVPA
LKEVSIADADGLFMVTTAVIIRDKYVRNVSCSVNNTLLGQEKETVIEG
RMDDISSTMVRSDKTHTCPPCPAPELLGGPSVFLFPPKPKDTLMISRT
PEVTCVVVDVSHEDPEVKFNWYVDGVEVHNAKTKPREEQYNSTYRVVS
VLTVLHQDWLNGKEYKCKVSNKALPAPIEKTISKAKGQPREPQVYTLP
PSRDELTKNQVSLTCLVKGFYPSDIAVEWESNGQPENNYKTTPPVLDS
DGSFFLYSKLTVDKSRWQQGNVFSCSVMHEALHNHYTQKSLSLSPGK
Mutant Amino acid sequence with long-acting
protein
(SEQ ID NO: 2)
(237aa BTN2A2 EC domain (bold and italicized) +
2 aa vector cloning seq + 227aa Fc region
(bold), underline shows the aa substitutions)
MEPAAALHFSLPASLLLLLLLLLLSLCALVSAQFTVVGPANPILAMVG
ENTTLRCHLSPEKNAEDMEVRWFRSQFSPAVFVYKGGRERTEEQMEEY
RGRITFVSKDINRGSVALVIHNVTAQENGIYRCYFQEGRSYDEAILRL
VVAGLGSKPLIEIKAQEDGSIWLECISGGWYPEPLTVWRDPYGEVVPA
LKEVSIADADGLFMVTTAVIIRDKYVRNVSCSVNNTLLGQEKETVRSD
KTHTCPPCPAPELLGGPSVFLFPPKPKDTLMISRTPEVTCVVVDVSHE
DPEVKFNWYVDGVEVHNAKTKPREEQYNSTYRVVSVLTVLHQDWLNGK
EYKCKVSNKALPAPIEKTISKAKGQPREPQVYTLPPSRDELTKNQVSL
TCLVKGFYPSDIAVEWESNGQPENNYKTTPPVLDSDGSFFLYSKLTVD
KSRWQQGNVFSCSVLHEALHSHYTQKSLSLSPGK
(BTN2A2 EC domain)
SEQ ID NO: 3
MEPAAALHFSLPASLLLLLLLLLLSLCALVSAQFTVVGPANPILAMVG
ENTTLRCHLSPEKNAEDMEVRWFRSQFSPAVFVYKGGRERTEEQMEEY
RGRITFVSKDINRGSVALVIHNVTAQENGIYRCYFQEGRSYDEAILRL
VVAGLGSKPLIEIKAQEDGSIWLECISGGWYPEPLTVWRDPYGEVVPA
LKEVSIADADGLFMVTTAVIIRDKYVRNVSCSVNNTLLGQEKETV
(Fc region (wt))
SEQ ID NO: 4
DKTHTCPPCPAPELLGGPSVFLFPPKPKDTLMISRTPEVTCVVVDVSH
EDPEVKFNWYVDGVEVHNAKTKPREEQYNSTYRVVSVLTVLHQDWLNG
KEYKCKVSNKALPAPIEKTISKAKGQPREPQVYTLPPSRDELTKNQVS
LTCLVKGFYPSDIAVEWESNGQPENNYKTTPPVLDSDGSFFLYSKLTV
DKSRWQQGNVFSCSVMHEALHNHYTQKSLSLSPGK
(Fc region (mutant))
SEQ ID NO: 5
DKTHTCPPCPAPELLGGPSVFLFPPKPKDTLMISRTPEVTCVVVDVSH
EDPEVKFNWYVDGVEVHNAKTKPREEQYNSTYRVVSVLTVLHQDWLNG
KEYKCKVSNKALPAPIEKTISKAKGQPREPQVYTLPPSRDELTKNQVS
LTCLVKGFYPSDIAVEWESNGQPENNYKTTPPVLDSDGSFFLYSKLTV
DKSRWQQGNVFSCSVLHEALHSHYTQKSLSLSPGK

In various embodiments, the present invention provides pharmaceutical compositions including a pharmaceutically acceptable excipient along with a therapeutically effective amount of the butyrophilin A2 (BTN2A2), a BTN2A2 fragment, a BTN2A2-related isoform, or a BTN2A2-related isoform fragment, the fusion protein, or the modified polypeptide described herein. “Pharmaceutically acceptable excipient” means an excipient that is useful in preparing a pharmaceutical composition that is generally safe, non-toxic, and desirable, and includes excipients that are acceptable for veterinary use as well as for human pharmaceutical use. Such excipients may be solid, liquid, semisolid, or, in the case of an aerosol composition, gaseous.

In certain embodiments, the compounds of the present invention may contain one or more acidic functional groups and, thus, are capable of forming pharmaceutically acceptable salts with pharmaceutically acceptable bases. The term “pharmaceutically acceptable salts, esters, amides, and prodrugs” as used herein refers to those carboxylate salts, amino acid addition salts, esters, amides, and prodrugs of the compounds of the present invention which are, within the scope of sound medical judgment, suitable for use in contact with the tissues of patients without undue toxicity, irritation, allergic response, and the like, commensurate with a reasonable benefit/risk ratio, and effective for their intended use of the compounds of the invention. The term “salts” refers to the relatively non-toxic, inorganic and organic acid addition salts of compounds of the present invention. These salts can be prepared in situ during the final isolation and purification of the compounds or by separately reacting the purified compound in its free base form with a suitable organic or inorganic acid and isolating the salt thus formed. These may include cations based on the alkali and alkaline earth metals such as sodium, lithium, potassium, calcium, magnesium and the like, as well as nontoxic ammonium, quaternary ammonium, and amine cations including, but not limited to ammonium, tetramethylammonium, tetraethyl ammonium, methyl amine, dimethyl amine, trimethylamine, triethylamine, ethylamine, and the like (see, e.g., Berge S. M., et al. (1977) J. Pharm. Sci. 66, 1, which is incorporated herein by reference).

The term “pharmaceutically acceptable esters” refers to the relatively nontoxic, esterified products of the compounds of the present invention. These esters can be prepared in situ during the final isolation and purification of the compounds, or by separately reacting the purified compound in its free acid form or hydroxyl with a suitable esterifying agent. Carboxylic acids can be converted into esters via treatment with an alcohol in the presence of a catalyst. The term is further intended to include lower hydrocarbon groups capable of being solvated under physiological conditions, e.g., alkyl esters, methyl, ethyl and propyl esters.

As used herein, “pharmaceutically acceptable salts or prodrugs” are salts or prodrugs that are, within the scope of sound medical judgment, suitable for use in contact with the tissues of subject without undue toxicity, irritation, allergic response, and the like, commensurate with a reasonable benefit/risk ratio, and effective for their intended use.

The term “prodrug” refers to compounds that are rapidly transformed in vivo to yield the functionally active one or more peptides as disclosed herein or a mutant, variant, analog or derivative thereof. As used herein, a prodrug is a compound that, upon in vivo administration, is metabolized or otherwise converted to the biologically, pharmaceutically or therapeutically active form of the compound. A prodrug of the one or more peptides as disclosed herein or a mutant, variant, analog or derivative thereof can be designed to alter the metabolic stability or the transport characteristics of one or more peptides as disclosed herein or a mutant, variant, analog or derivative thereof, to mask side effects or toxicity, to improve the flavor of a compound or to alter other characteristics or properties of a compound. By virtue of knowledge of pharmacodynamic processes and drug metabolism in vivo, once a pharmaceutically active form of the one or more peptides as disclosed herein or a mutant, variant, analog or derivative thereof, those of skill in the pharmaceutical art generally can design prodrugs of the compound. Suitable examples of prodrugs include methyl, ethyl and glycerol esters of the corresponding acid.

In various embodiments, the pharmaceutical compositions according to the invention may be formulated for delivery via any route of administration. “Route of administration” may refer to any administration pathway known in the art, including but not limited to aerosol, nasal, oral, transmucosal, transdermal or parenteral. “Transdermal” administration may be accomplished using a topical cream or ointment or by means of a transdermal patch. “Parenteral” refers to a route of administration that is generally associated with injection, including intraorbital, infusion, intraarterial, intracapsular, intracardiac, intradermal, intramuscular, intraperitoneal, intrapulmonary, intraspinal, intrasternal, intrathecal, intrauterine, intravenous, subarachnoid, subcapsular, subcutaneous, transmucosal, or transtracheal. Via the parenteral route, the compositions may be in the form of solutions or suspensions for infusion or for injection, or as lyophilized powders. Via the enteral route, the pharmaceutical compositions can be in the form of tablets, gel capsules, sugar-coated tablets, syrups, suspensions, solutions, powders, granules, emulsions, microspheres or nanospheres or lipid vesicles or polymer vesicles allowing controlled release. Via the parenteral route, the compositions may be in the form of solutions or suspensions for infusion or for injection. Via the topical route, the pharmaceutical compositions based on compounds according to the invention may be formulated for treating the skin and mucous membranes and are in the form of ointments, creams, milks, salves, powders, impregnated pads, solutions, gels, sprays, lotions or suspensions. They can also be in the form of microspheres or nanospheres or lipid vesicles or polymer vesicles or polymer patches and hydrogels allowing controlled release. These topical-route compositions can be either in anhydrous form or in aqueous form depending on the clinical indication. When administered via the ocular route, they may be in the form of eye drops.

The pharmaceutical compositions according to the invention can also contain any pharmaceutically acceptable carrier. “Pharmaceutically acceptable carrier” as used herein refers to a pharmaceutically acceptable material, composition, or vehicle that is involved in carrying or transporting a compound of interest from one tissue, organ, or portion of the body to another tissue, organ, or portion of the body. For example, the carrier may be a liquid or solid filler, diluent, excipient, solvent, or encapsulating material, or a combination thereof. Each component of the carrier must be “pharmaceutically acceptable” in that it must be compatible with the other ingredients of the formulation. It must also be suitable for use in contact with any tissues or organs with which it may come in contact, meaning that it must not carry a risk of toxicity, irritation, allergic response, immunogenicity, or any other complication that excessively outweighs its therapeutic benefits.

The pharmaceutical compositions according to the invention can also be encapsulated, tableted or prepared in an emulsion or syrup for oral administration. Pharmaceutically acceptable solid or liquid carriers may be added to enhance or stabilize the composition, or to facilitate preparation of the composition. Liquid carriers include syrup, peanut oil, olive oil, glycerin, saline, alcohols and water. Solid carriers include starch, lactose, calcium sulfate, dihydrate, terra alba, magnesium stearate or stearic acid, talc, pectin, acacia, agar or gelatin. The carrier may also include a sustained release material such as glyceryl monostearate or glyceryl distearate, alone or with a wax.

The pharmaceutical preparations are made following the conventional techniques of pharmacy involving milling, mixing, granulation, and compressing, when necessary, for tablet forms; or milling, mixing and filling for hard gelatin capsule forms. When a liquid carrier is used, the preparation will be in the form of a syrup, elixir, emulsion or an aqueous or non-aqueous suspension. Such a liquid formulation may be administered directly p.o. or filled into a soft gelatin capsule.

The pharmaceutical compositions according to the invention may be delivered in a therapeutically effective amount. The precise therapeutically effective amount is that amount of the composition that will yield the most effective results in terms of efficacy of treatment in a given subject. This amount will vary depending upon a variety of factors, including but not limited to the characteristics of the therapeutic compound (including activity, pharmacokinetics, pharmacodynamics, and bioavailability), the physiological condition of the subject (including age, sex, disease type and stage, general physical condition, responsiveness to a given dosage, and type of medication), the nature of the pharmaceutically acceptable carrier or carriers in the formulation, and the route of administration. One skilled in the clinical and pharmacological arts will be able to determine a therapeutically effective amount through routine experimentation, for instance, by monitoring a subject's response to administration of a compound and adjusting the dosage accordingly. For additional guidance, see Remington: The Science and Practice of Pharmacy (Gennaro ed. 20^{th} edition, Williams & Wilkins PA, USA) (2000).

### Kits

The present invention is also directed to a kit. Thus, in some embodiments the kit contains a composition including: a butyrophilin A2 (BTN2A2), a BTN2A2 fragment, a BTN2A2-related isoform, or a BTN2A2-related isoform fragment, the fusion protein, or the modified polypeptide as described herein.

The exact nature of the components configured in the inventive kit depends on its intended purpose. In one embodiment, the kit is configured particularly for the purpose of treating mammalian subjects. In another embodiment, the kit is configured particularly for the purpose of treating human subjects. In further embodiments, the kit is configured for veterinary applications, treating subjects such as, but not limited to, farm animals, domestic animals, and laboratory animals. In another embodiments, the kit is configured for detecting a butyrophilin A2 (BTN2A2), a BTN2A2 fragment, a BTN2A2-related isoform, or a BTN2A2-related isoform fragment.

Instructions for use may be included in the kit. “Instructions for use” typically include a tangible expression describing the technique to be employed in using the components of the kit to effect a desired outcome. Optionally, the kit also contains other useful components, such as, diluents, buffers, pharmaceutically acceptable carriers, syringes, catheters, applicators, pipetting or measuring tools, bandaging materials or other useful paraphernalia as will be readily recognized by those of skill in the art.

The materials or components assembled in the kit can be provided to the practitioner stored in any convenient and suitable ways that preserve their operability and utility. For example the components can be in dissolved, dehydrated, or lyophilized form; they can be provided at room, refrigerated or frozen temperatures. The components are typically contained in suitable packaging material(s). As employed herein, the phrase “packaging material” refers to one or more physical structures used to house the contents of the kit, such as inventive compositions and the like. The packaging material is constructed by well-known methods, preferably to provide a sterile, contaminant-free environment. As used herein, the term “package” refers to a suitable solid matrix or material such as glass, plastic, paper, foil, and the like, capable of holding the individual kit components. Thus, for example, a package can be a glass vial used to contain suitable quantities of an inventive composition containing: a butyrophilin A2 (BTN2A2), a BTN2A2 fragment, a BTN2A2-related isoform, or a BTN2A2-related isoform fragment, the fusion protein, or the modified polypeptide as described herein. In another example, a package can be a glass vial used to contain suitable quantities of an inventive composition containing: an anti-butyrophilin A2 (BTN2A2) antibody, an anti-BTN2A2 fragment antibody, an anti-BTN2A2-related isoform antibody, or an antiBTN2A2-related isoform fragment antibody described herein. The packaging material generally has an external label which indicates the contents and/or purpose of the kit and/or its components.

### EXAMPLES

The following examples are provided to better illustrate the claimed invention and are not to be interpreted as limiting the scope of the invention. To the extent that specific materials are mentioned, it is merely for purposes of illustration and is not intended to limit the invention. One skilled in the art may develop equivalent means or reactants without the exercise of inventive capacity and without departing from the scope of the invention.

### Example 1

### Materials and Methods

### BTN2A2-Fc Construct Generation and Purification of Recombinant Protein

Soluble recombinant BTN2A2-Fc protein was generated using baculovirus infected insect cell system, a method that is both scalable and has been successfully used for generation of recombinant proteins in the immune system. An upstream 711 bp region of human BTN2A2 gene (NP_008926.2) containing signal peptide and two extracellular domains (IgV and IgC2) was amplified using specific primers (See Table 1) from cDNA from 293T cells. Amplified region was first cloned into pFUSE-hIgG1-Fc1 vector (InvivoGen Catalog #pfuse-hg1fc1) using Age1 and EcoRV restriction sites. Subsequently, BTN2A2 region along with Fc region was amplified using specific primers (Table 1) and cloned into pBacPak8 vector using Xba1 and Sac1 restriction sites. Paired-end sequencing of the clone was performed by sanger sequencing to confirm the sequence. Primers were synthesized from Integrated DNA Technologies, Inc (IDT) and Phusion® High-Fidelity PCR Master Mix with HF Buffer was used in all PCR amplification reaction (New England Biolabs). Baculovirus were generated by co-transfecting pBacPak8-BTN2A2-Fc clone with linearized baculovirus DNA (TakaraBio Catalog #631401) into Sf9 insect cells according to manufacturer protocol. Preparation of baculovirus passage (P0, P1, P2) was done as described in manufactures protocol (TakaraBio, Cat #631402). P3 baculovirus stock was used for large scale protein purification using Hi5 cells grown in serum free media (Express Five™ SFM, ThermoFisher, Cat #10486025). Pierce™ Protein G Agarose columns (ThermoFisher Scientific, Cat #20398) was used for purification of secreted soluble-BTN2A2-Fc protein from the supernatant. Purified protein was finally suspended in 1× phosphate buffer saline (1×PBS). Protein purity was checked by SDS-PAGE followed by Coomassie blue staining. Specificity of the purified protein was confirmed by western blot analysis using specific BTN2A2 antibody. Commercially available recombinant 293 cells derived human BTN2A2-Fc (Cat #8918-BT: R&D systems) and mouse BTN2A2 (Cat #8997-BT-050: R&D systems) was used for comparison studies with baculoviral-generated recombinant B2N2A2-Fc.

TABLE 1
Primer Sequence

| | | SEQ |
|---|---|---|
| Primer name | Primer Sequence | ID NO |
| BTN2A2-AgeI-1F | ATAT-ACCGGT- | 8 |
| | ATGGAACCAGCTGCTGCTC | |
| BTN2A2-EcoRV- | ATAT-GATATC- | 9 |
| IEGRMD (SEQ ID | GTCCATACGTCCCTCGAT- | |
| NO: 57)-711R | GACAGTTTCCTTCTCCTGGCC | |
| BTN-Xbal-F | ATAT-TCTAGA-ATGGAACCAGCTG | 10 |
| | CTGCTC | |
| BTN-SacI-R | ATAT-GAGCTC-ACTCATTTACCCG | 11 |
| | GAGACAG | |
| Mus-GAPDH-qF2 | ACCACAGTCCATGCCATCAC | 12 |
| Mus-GAPDH-qR2 | TCCACCACCCTGTTGCTGTA | 13 |
| Mus-ActinB-qF1 | CGCCACCAGTTCGCCATGGA | 14 |
| Mus-ActinB-qR1 | TACAGCCCGGGGAGCATCGT | 15 |
| Mus-Foxp3-F2 | CCTGGTTGTGAGAAGGTCTTCG | 16 |
| Mus-Foxp3-R2 | TGCTCCAGAGACTGCACCACTT | 17 |
| Mus-RORC-qF1 | CACGGCCCTGGTTCTCAT | 18 |
| Mus-RORC-qR1 | GCAGATGTTCCACTCTCCTCTTCT | 19 |
| cyBTNKOms-R1 | AAAGATGGGGAGAGAAAGTCAAGAG | 20 |
| cyBTNKOms-F1 | CCTACCTAGTTTTACCTGAGGTTC | 21 |
| cyBTNKOms-F2 | GGTCCATTGATATCCAAAGCCAAA | 22 |
| IL2RB-mus-qF1 | CTCAAGTGCCACATCCCAGATC | 23 |
| IL2RB-mus-qR1 | AGCACTTCCAGCGGAGAGATCT | 24 |
| IL21-mus-qF1 | GCCTCCTGATTAGACTTCGTCAC | 25 |
| IL21-mus-qR1 | CAGGCAAAAGCTGCATGCTCAC | 26 |
| GATA3-Mus-qF1 | CCTCTGGAGGAGGAACGCTAAT | 27 |
| GATA3-Mus-qR1 | GTTTCGGGTCTGGATGCCTTCT | 28 |
| Tbx21-mus-qF1 | CCACCTGTTGTGGTCCAAGTTC | 29 |
| Tbx21-mus-qR1 | CCACAAACATCCTGTAATGGCTTG | 30 |
| SMAD3-mus-qF1 | GCTTTGAGGCTGTCTACCAGCT | 31 |
| SMAD3-mus-qR1 | GTGAGGACCTTGTCAAGCCACT | 32 |
| CD5-mus-qF1 | AGAACCAGGTCTTCTGCCAAGG | 33 |
| CD5-mus-qR1 | TTGTGGGTGGAGGTGTCGTTCT | 34 |
| Cr-CD45-g1F | caccGGTTTGCAGAATTACCACAA | 35 |
| Cr-CD45-g1R | aaacTTGTGGTAATTCTGCAAACC | 36 |
| Cr-CD45-g2F | caccGTAATATTTACCCACCACCC | 37 |
| Cr-CD45-g2R | aaacGGGTGGTGGGTAAATATTAC | 38 |

### T-Cells Activation and IL-2 Secretion Assay

In vitro T cell activation was performed. Briefly, 96-well plates were coated with 1 μg/ml concentrations of anti-CD3 mAb (OKT3, clone x) in PBS at 4° C. overnight in absence or presence of recombinant BTN2A2-Fc (10 μg/ml) or Fc tag protein (Cat #10702-HNAH, Sino Biological Inc) or 293 cells derived human BTN2A2-Fc or mouse BTN2A2. A total of 2.5×10⁵ Jurkat cells/well were added to precoated flat-bottom 96-well plates. Cells were incubated for 24 hrs in 37° C. incubator with 5% CO₂. Total 100 μl of medial was used to measure IL-2 production using an IL-2 ELISA kit from Millipore-Sigma (Cat #RAB0286-1KT) according to the manufacturer' protocol.

### In Vitro TCR Stimulation and Signal Transduction Analysis

For short-term activation of the Jurkat cells, a 48-well tissue culture plate was coated with anti-CD3 mAb (OKT3, 10 μg/mL) and BTN2A2-Fc (10 μg/mL) or Fc-Tag (10 μg/mL). Plate was incubated overnight at 4° C. and each well was washed twice with PBS. A total of 2×10⁶ cells in 75 μl 1×PBS were then added to each well for 3 min at 37° C. The reaction was then stopped with ice cold PBS. Pervanadate was prepared by incubating vanadate (200 mM) and H₂O₂ (200 mM) in 1:2 ratio for 15 min at room temperature. Jurkat cells in 1×PBS were treated with pervanadate at final concentration of 0.1 mM for 5 min in incubator (5% CO2 and 37° C.). The cells were then collected, lysed in lysis buffer (RIPA or IP lysis buffer) and subsequently subjected to immunoprecipitation reaction or immunoblotting.

### Immunoprecipitation and Immunoblotting

Jurkat cells were activated with plate-bound anti-CD3 BTN2A2-Fc or Fc-tag protein as control or left un-activated. Cells were collected then washed in PBS and resuspended in IP lysis buffer (10 mM HEPES pH 7.5, 0.5 mM EDTA, 0.5% NP-40, 250 mM NaCl, 1× phosSTOP and protease inhibitors), incubated on ice for 30 min with vortexing, and cleared by centrifugation at 15,000 g for 10 min. An aliquot of protein lysate was used for western blotting, while the remainder were incubated for 4 hrs at 4° C. with specific antibodies or IgG control. Protein A/G Agarose (ThermoFisher Scientific) was added to lysate and incubated for additional 2 hrs. Beads were washed once in IP-lysis buffer with 0.5% NP-40 and twice in IP-lysis buffer with 0.2% NP-40. Proteins were eluted with SDS sample buffer supplemented with DTT by heating at 70° C. for 5-10 min. For Western blot analysis, eluted fractions from un-activated cells, cells co-activated with plate-bound anti-CD3 antibody and BTN2A2-Fc, or cells co-activated with anti-CD3 antibody and Fc-tag control, and their respective input controls were blotted as described below. Approximately 12×10⁶ Jurkat cells were used for each condition and lysed in 300 ml IP-lysis buffer. For immunoprecipitation, lysate was incubated with 1 mg of antibody to specific protein or respective IgG control. Protein A/G Agarose beads were used to pulldown the complex and eluted in 100 ul sample buffer. Western blots were probed with anti-phospho-Zap70 anti-phospho-CD3z antibody and also antibody against total Zap70 and CD3z protein (See Table 2). For phosphatase activity, immunoprecipitated material was directly mixed in Fluorescein Diphosphate, Tetraammonium Salt substrate (FDP, Catalog #F2999, Thermofisher Scientific) and activity was measured using Molecular Devices SpectraMax® M2 plate readers as recommended by protocol included by manufacturer.

TABLE 2
Antibody List

| Vendor name | Name |
|---|---|
| Novus Biologicals | BTN2A2/Butyrophilin 2 Antibody (6C7D2) |
| R&D system | Human BTN2A2/Butyrophilin 2A2 Antibody |
| Cell Signaling Tech | CD45 (Intracellular Domain) (D9M8I) XP ® Rabbit mAb |
| Abcam | CD45 antibody [MEM-28] (ab8216) |
| ThermoFisher | CD45RA Monoclonal Antibody (MEM-56) |
| Scientific | |
| ThermoFisher | CD45RB Recombinant Rabbit Monoclonal |
| Scientific | Antibody (PTPRC, 2877R) |
| ThermoFisher | CD45RO Monoclonal Antibody (UCHL1) |
| Scientific | |
| Cell Signaling Tech | IL-17A (D1X7L) Rabbit mAb (Mouse Specific) |
| R&D system | Mouse CD3ε Antibody |
| BD BioScience | Purifed NA/LE Mouse Anti-Human CD3 |
| BD BioScience | Purified NA/LE Hamster Anti-Mouse CD28 |
| BD BioScience | Purified NA/LE Rat Anti-Mouse CD3 Molecular Complex |
| Cell Signaling Tech | rabbit IgG, HRP-linked Antibody |
| ThermoFisher | Rat Serum |
| Scientific | |
| R&D system | Normal Rabbit IgG Control |
| Sigma | IgG from human serum |
| R&D system | Mouse IgG2B Isotype Control |
| Sino-Biological | IgG1 Fc Protein, Human, Recombinant (C103S) |
| Abcam | Recombinant Anti-CD3 epsilon antibody |
| Abcam | Recombinant Anti-CD3 zeta (phospho Y142) antibody |
| R&D system | Human Lck Antibody |
| Cell Signaling Tech | Zap-70 (99F2) Rabbit mAb |
| Cell Signaling Tech | Phospho-Zap-70 (Tyr319)/Syk (Tyr352) (65E4) Rabbit mAb |
| Abcam | CD3 zeta antibody |
| Cell Signaling Tech | GAPDH (14C10) Rabbit |
| Cell Signaling Tech | Anti-mouse IgG, HRP-linked Antibody |
| ThermoFisher | Donkey anti-Mouse IgG Antibody, Alexa Fluor ™ 555 |
| Scientific | |
| ThermoFisher | Goat anti-Rabbit IgG Antibody, Alexa Fluor ™ 488 |
| Scientific | |
| BD Bioscience | FITC Hamster Anti-Mouse CD3e |
| BD Bioscience | FITC Mouse Anti-Human CD3 Clone UCHT1 (also known as UCHT- 1; |
| | UCHT 1) (RUO) |
| BD Bioscience | APC Mouse Anti-Human CD4 Clone RPA-T4 (RUO) |
| BD Bioscience | APC Rat Anti-Mouse CD4 |
| Biolegend | Brilliant Violet 711 ™ anti-mouse CD25 Antibody |
| BD BioScience | BV421 Mouse Anti-Mouse RORγ |
| Biolegend | PE anti-mouse CD3 Antibody |
| BD Bioscience | PE Mouse Anti-Human CD3 Clone OKT3 (RUO) |
| BD Bioscience | PE Mouse anti-Human FoxP3 Clone 259D/C7 (RUO) |
| BD BioScience | PE Mouse anti-Human RORγt |
| BD BioScience | PE Mouse anti-Mouse RORγt |
| BD BioScience | PE Rat Anti-Mouse CD3 Molecular Complex |
| BD Bioscience | PE Rat anti-Mouse Foxp3 |
| BD BioScience | Human BD Fc Block |
| BD BioScience | Purified Rat Anti-Mouse CD16/CD32 (Mouse BD Fc Block ™) |
| BD BioScience | PE Mouse IgG2a, κ Isotype Control |
| BD BioScience | PE Rat IgG1, κ Isotype Control |
| BD Biosciences | PE Rat IgG2b, κ Isotype Control |

Extracellular crosslinking with BS3 (bis[sulfosuccinimidyl] suberate) was performed according to user manual (Thermo Scientific MAN0011240). Jurkat cells were washed with PBS and then incubated on ice with 10 μg/mL BTN2A2-Fe and Fe-tag protein for 1 hr. To induce crosslinking, BS3 (ThermoFisher) was added at a final concentration of 5 mM and incubated for 30 min at room temperature. The reaction was stopped using Tris HCl, pH 7.5, at a final concentration of 20 mM for 15 minutes at room temperature. The cells were then washed extensively in PBS and resuspended in IP-lysis buffer and cleared by centrifugation at 14,000 g for 10 min. Protein G Agarose beads were used to pulldown BTN2A2-Fc and Fc-tagged protein. Beads were washed and eluted with SDS sample buffer supplemented with dithiothreitol. Eluted samples were analyzed by western blot probed with anti-CD45 antibody.

For Western blot analysis, Jurkat cells were collected and centrifuged. Cell pellets were resuspended in 2.5× volume of RIPA buffer (20 mM Tris pH 7.5, 150 mM NaCl. 1 mM EDTA, 1% NP-40, 0.5% sodium deoxycholate, and 0.5% SDS containing protease and phosphatase inhibitors). Total protein concentrations in the lysates were determined using the Pierce™ BCA Protein Assay Kit (ThermoFisher Scientific) by measuring absorption at 595 nm on the microplate reader (Bio-Rad). Total of 40-80 μg of each protein sample was used for western blot analysis. Protein lysate prepared after Jurkat cell treatment and/or immunoprecipitation was separated in 4-12% Bis-Tris gel (ThermoFisher Scientific) and transferred to nitrocellulose membrane. Membrane was blocked for 1 h in blocking solution (5% BSA in PBS with 0.1% Tween [PBST]). The membrane was incubated with primary antibodies overnight at 4° C. washed with PBST three times (10 minutes each), incubated with secondary antibodies for 1 h at room temperature, and finally washed with PBST three times (10 minutes each). The blots were visualized using an ECL assay (ThermoFisher Scientific). Antibodies used for immunoblotting analysis are listed in Table 1.

### Molecular Modeling of BTN2A2 and PTPRC

A molecular model of BTN2A2 was generated using homology modeling. A search for the homologous structure of the extracellular domain of BTN2A2 revealed that BTN3A2 shares about 47% of sequence homology with BTN2A2. Using the crystal structure of BTN3A2 as a template, the three-dimensional model of BTN2A2 was generated using SWISS-MODEL workspace. Subsequently, the molecular structure of BTN2A2 was subjected to a short 1.2 ns molecular dynamics simulation using Desmond (Schrodinger, Inc., San Diego, CA). The three-dimensional structure of PTPRC was retrieved from the protein data bank (PDB code: 5FN7). To determine the interaction between BTN2A2 and PTPRC proteins, RosettaDock 4.0 was used. The top 10 predicted models were then subjected to 1.2 ns molecular simulation followed by minimization using Desmond. Then, the most energetically stable docking model was used to identify potential amino acids for mutation.

### CRISPR-Cas9 Deletion of CD45 Gene and Site Directed Mutagenesis of CD45

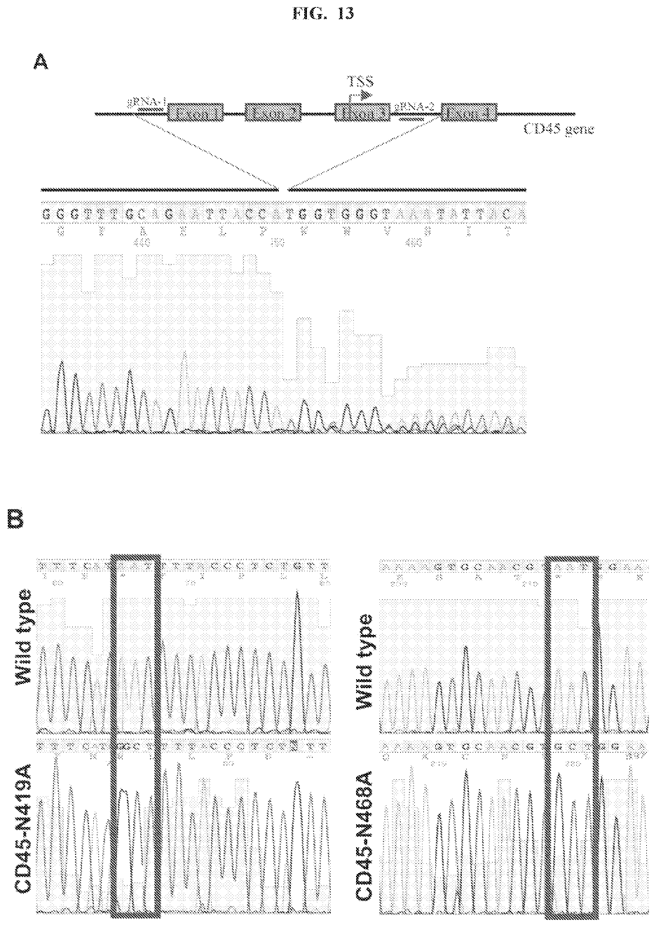

CD45 knockdown cell lines were generated using the CRISPR-Cas9 system. Two different guide RNAs were designed complementary to upstream of CD45 exon-1 and downstream to exon-3 based on its protospacer adjacent motif (PAM) sequence (Table 2). These guide RNA sequences were cloned into lenti-Crispr v2 blast vector containing the Cas9 coding gene and a blasticidine resistant gene. Lentivirus were generated using envelop and packaging plasmid. Jurkat cells were then transduced with lentivirus expressing guideRNA and Cas9 gene and selected for blasticidine resistant cells. Single-cell cloning was performed to isolate independent clones. Deletion of Exon-1 to Exon-3 was confirmed by direct Sanger sequencing (FIG. 13A).

CD45 (PTPRC) (NM_002838) Human Tagged ORF Clone was purchased from origene technologies. Inc. Site-directed mutagenesis were performed using the QuickChange mutagenesis kit (Invitrogen), to introduce the N419A and N468A mutations. Introduction of the mutation was confirmed by direct sequencing (FIG. 13B). Lentivirus were generated using envelop and packaging plasmid. Jurkat cells deleted for CD45 gene were then transduced with lentivirus expressing mutant CD45 gene.

### In Vitro Treg and Th17 Differentiation by Mixed Lymphocyte Reaction (MLR)

For Treg differentiation, fresh spleen, and lymph nodes (inguinal, brachial and axillary) were harvested from mice and washed with 1×RPMI media. Spleen was excised into small pieces and single cells were prepared by mashing the tissue with the plunger end of syringe through a 70 micron strainer and cells were suspended in 5 ml of RPMI media. Cells were stimulated with plate bound anti-CD3 (1 μg/ml) with or without BTN2A2-Fe fusion protein (10 μg/ml) and co-cultured with CD1 mice irradiated splenocytes for 7 days. Cells were labeled with immunofluorescent antibodies and analyzed for CD4+CD25+Foxp3−GFP+ve co-expression using flow cytometry. CD4+ve cells incubated with anti-CD3 antibody in absence or presence of recombinant BTN2A2-Fc for day-1 and day-5 were used to extract RNA for qPCR analysis. CD4+ve T cells were isolated using EasySep™ Mouse CD4+ T Cell Isolation Kit (Stem Cell Tech); B cells using EasySep™ Mouse B Cell Isolation Kit (Stem Cell Tech); Pan-DC cells using EasySep™ Mouse Pan-DC Cell Enrichment Kit II (Stem Cell Tech); T cells using EasySep™ Mouse T Cell Isolation Kit (Stem Cell Tech) per protocols recommended by manufacturer.

To explore effects of BTN2A2 on Th17 differentiation, 1×10⁶ spleen cells were stimulated with soluble anti-CD3 antibody (0.5 mg/ml) and CD1 mice irradiated spleen cells (1:1 ratio) and Th17 differentiation cytokines cocktail (TGF-b1 1.5 ng/ml, IL-6 10 ng/ml, IL-1b 10 ng/ml) with or without recombinant BTN2A2-Fc (10 μg/ml). Cells were co-cultured for 5 days and subjected to intracellular immunofluorescence staining of RORgt (Th17 cell marker). Cells were analyzed for CD4+RORgt+ve co-expression using flow cytometry. CD45 phosphatase inhibitor (Compound 211, Catalog #530197; Millipore Sigma) that has previously been characterized as irreversible, and selective blocker of the allosteric pocket at the D1-D2 domains interface away from the substrate-binding/catalytic site, was used at 125 nM concentration during the course of T cell differentiation studies.

### Flow Cytometry Analysis

Immune cells were freshly harvested from spleen and lymph nodes (inguinal, brachial and axillary) of mice. Cells were counted using a Hemavet 950FS hematology analyzer (Drew Scientific, Miami Lake, FL). Immunostaining and flow cytometry analysis of cells were performed as described previously (Li et al., 2018). Briefly, cells isolated from mice or treated in vitro cultures were harvested and stained with Ghost Dye™ Red 780 Viability Dye (Cell Signaling). Cells were washed extensively with 0.5% BSA in 1×PBS and resuspended in staining buffer (FBS); and further stained with fluorochrome conjugated antibodies for cells surface marker anti-CD3, anti-CD4 and anti-CD25. Cells were washed with staining buffer to remove excess antibodies and then fixed/permeabilized using mouse Foxp3 Buffer Set (BD Biosciences, San Jose, CA) according to manufacturer protocol. Fixed/permeabilized cells were incubated in rat serum for 15 minutes and stained with intracellular markers (Foxp3 and/or RORgt). Cells harvested from Foxp3−GFP transgenic mice were stained with anti-CD3, anti-CD4 and anti-CD25 antibodies, washed and suspended buffer containing DAPI. Cells were directly analyzed for CD4+CD25+Foxp3−GFP or CD4+RORgt expression. The stained cells were analyzed using a BD LSR Fortessa (BD Biosciences. San Jose, CA). Details of the commercial antibodies used in this study are provided in Table 1.

### Cells Proliferation and Apoptosis Assay

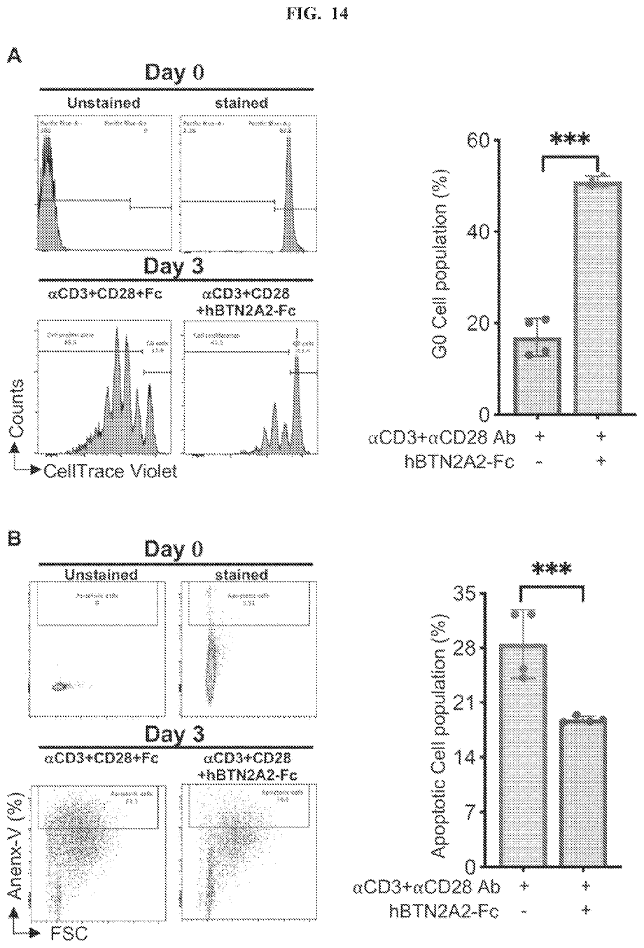

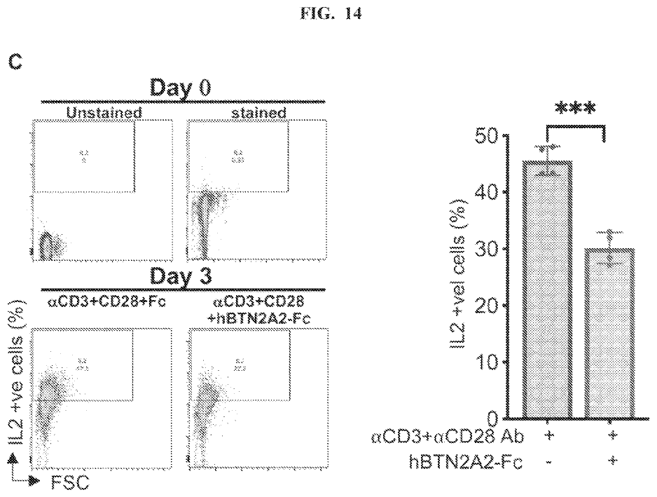

CD4+ cells were isolated from freshly harvested spleen of wild type B6 mice as described above. Cells were stained with CellTrace™ Violet dye at 5 mM dye concentration in 10⁶ cells/ml dilution according to manufacturer protocol (Invitrogen). 2.5×10⁶ CellTrace™ Violet stained cells were incubated on bound anti-CD3 (0.5 μg/ml) plus anti-CD28 (0.5 μg/ml) with BTN2A2-Fc fusion protein (10 μg/ml) or Fc control (10 μg/ml). Cells were culture for 3 days at 37° C. and 5% CO2 in RPMI medium supplemented with 10% FBS and 1% Pcn-Strp. Unstimulated parent generation (GO) indicated as the brightest peak on the far-right side of the histogram (FIG. 14). Cells were stained with Annexin V-FITC conjugate for apoptotic cells assay. For intracellular staining of IL2, cells were fixed, permeabilized and stained with IL2 antibody and analyzed using a BD LSR Fortessa (BD Biosciences, San Jose, CA).

### Immunofluorescence Studies

Immunofluorescence staining for CD45 and CD3ζ was performed. Anti-CD3 antibody (10 mg/ml) and recombinant BTN2A2-Fc (10 mg/ml) were coated on glass poly-L-lysine cover slips in PBS overnight at 4° C. 1×10⁶ Jurkat cells in 75 ul of 1×PBS were exposed to coated coverslip for 3 min at 37° C. and fixed using Cytofix/Cytoperm-Fixation/Permeabilization Kit (BD biosciences). Coverslips were then gently washed two times with 1×PBS. Cells were blocked with blocking buffer (5% goat/mouse serum in 0.3% triton-x100 in 1×PBS) and stained with mouse anti-human CD45 (abcam cat #ab8216) and rabbit anti-human CD3ζ (abeam cat #Ab226263). Coverslips were then gently washed 3 time with 1×PBS for 10 minutes each and stained with Donkey anti-Mouse IgG Antibody, Alexa Fluor™ 555 and Goat anti-Rabbit IgG Antibody, Alexa Fluor™ 488 (ThermoFisher Scientific). Coverslips were then gently washed 3 time with 1×PBS for 10 minutes each, incubated in DAPI for 5 minutes and mounted on slides using ProLong™ Diamond Antifade Mountant (ThermoFisher Scientific). Glass slides were then imaged with a Zeiss LSM 780 confocal microscope through a 63× resolution with oil immersion. The image was analysed as an 8-bit image and intensity of red and green channel was measured from 0-255 grayscale fluorescent units. Co-localization analysis was performed on Image-J plugin “Colocalization Finder”. At least seven fields were analysed per experimental condition, in which an average of 50 T cells were analysed to yield the average amount of co-localization.

### Quantitative Polymerase Chain Reaction (QPCR)

For gene expression analysis, the total RNA was extracted using TRIzol™ Reagent (Thermo Fisher Scientific). The cDNA was synthesized from 500 ng of total RNA using SuperScript™ IV VILO™ Master Mix (Thermo Fisher Scientific). Quantitative real time PCR was performed with PowerUp™ SYBR™ Green Master Mix (Thermo Fisher Scientific) on a QuantStudio Real-Time PCR system (Thermo Fisher Scientific). GAPDH was used as internal controls. Gene expression was compared using the ΔΔCt method. Primers details are provided in (Table 2).

### Animal Models

All animal experiments were conducted in accordance with the National Institutes of Health Guide for the Care and Use of Laboratory Animals and all the protocols used in this study were approved by the Cedars-Sinai Medical Center Institutional Animal Care and Use Committee.

Foxp3EGFP (C.Cg-Fox3^{tm2Tch}/J, Strain #006769) mice: Foxp3EGF mice that co-express EGFP and the regulatory T cell-specific transcription factor Foxp3 under the control of the endogenous promoter was acquired from the Jackson Laboratory.

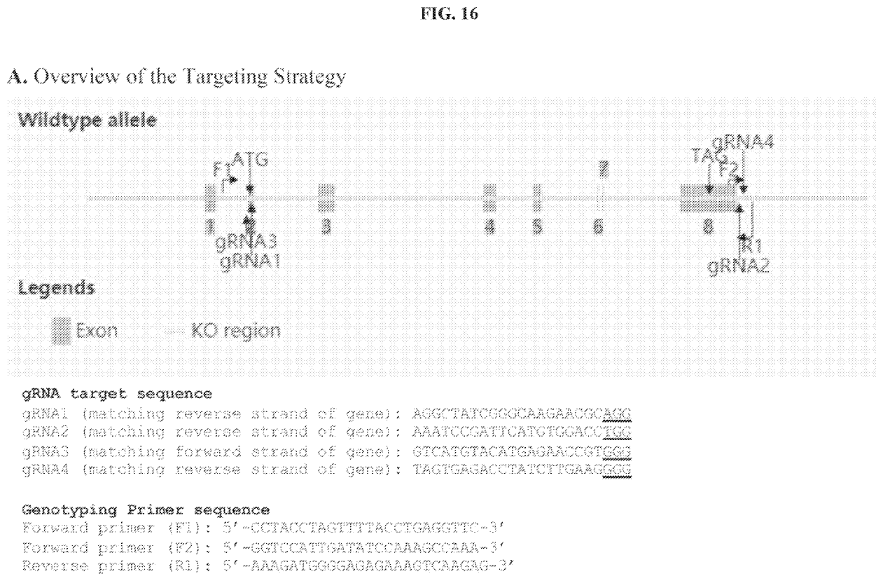

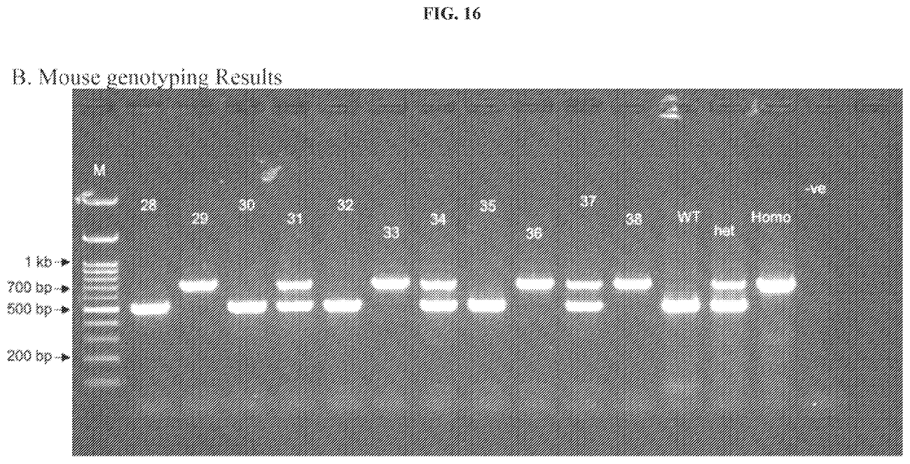

BTN2A2 Knock-out mice (−/−): BTN2A2L−/−Jmice (strain: C57BL/6J-Btn2a2em1cyagen) were generated by deleting 10460 base pair region of btn2a2 gene comprising exon2-8 using CRISPR-Cas9 technique at commercial facility (Cyagen Biosciences) (FIG. 16). Three PCRprimers set (Table 1) were used for genotyping mice using polymerase chain reaction method. Wildtype mice shows single band of 500 bp, heterozygous shows two bands of 700 bp and 500 bp, while homozygous mice show 500 bp single band on agarose gel electrophoresis (FIG. 16).

Glomerulonephritis Model: The detailed protocol to induce crescentic glomerulonephritis (GN) has been described previously. Nephrotoxic sera were raised in rabbits by repeated immunization with the purified glomeruli in complete and incomplete Freund's adjuvant. The mice were preimmunized with normal rabbit IgG and complete Freund's adjuvant five days prior to administration of nephrotoxic serum. Nephrotoxic serum nephritis was induced by the injection of 20 ml or 10 ml nephrotoxic serum intravenously at day 0. Four doses of 25 mg BTN2A2-Fc fusion protein or vehicle (control) were injected i.p. at day 0, 2, 4, 6 of nephrotoxic serum injection. Mice were scarified at day 7 to collect tissues, urine, blood cells and plasma. Immune cells were harvested from fresh whole spleen and lymph-nodes (Inguinal, Brachial and Axillary) and CD4+ve T cells were isolated using EasySep™ Mouse CD4+ T Cell Isolation Kit (STEM CELL TECH). Protein lysate of snap-freeze tissue in liquid nitrogen was prepared by homogenizing 5 mg tissue in 500 ul ice-cold RIPA lysis buffer. Lysate was agitated for 2 hrs at 4° C., centrifuged at 16000 g for 20 min at 4° C. and supernatant was collected. About 80 mg of tissue lysate was used to detect IL17A expression using western blot.

To obtain the protein-to-creatinine ratio (mg/mg) in urine for evaluation of proteinuria, urine protein was measured with a protein assay dye (No. 500-0006, Bio-Rad, Hercules, CA), and urinary creatinine was measured using a Creatinine Assay Kit (No. DICT-500, BioAssay Systems, Hayward, CA). Fibrinoid necrosis (a precursor lesion for crescents, defined by finding GBM rupture, fibrin deposition, and karyorrhexis), and crescents were assessed in all glomeruli on one paraffin section for each mouse using periodic acid-methenamine silver and PAS stains, respectively. Representative images were captured using a light microscope (Nikon Eclipse 50i, Nikon, Tokyo, Japan). Histopathologic diagnosis of GN was evaluated by a board-certified renal pathologist (M.Y.)

Miscarriage Model: DBA/2 and CBA/J mice were purchased from Jackson Laboratory. We crossed male DBA/2 with female CBA/J to document immunologically mediated pregnancy loss as described elsewhere (Zenclussen et al., 2005). As controls, we crossed female DBA/2 with male CBA/J strain and we evaluated litter size, pup weight and litter resorption rates. To study the therapeutic effects of BTN2A2, four doses of 25 mg BTN2A2-Fc fusion protein were injected intraperitoneally at gestational days 6, 9, 12 and 15 in CBA/J female mice that were crossed with male DBA/2 mice. Animals were sacrificed at gestational day 18 and blood/tissues harvested for molecular analysis.

### Statistics

Data were graphed and statistics performed using GraphPad Prism version 9.2. Data are presented mean f standard deviation (SD) as indicated. Shapiro-Wilk test was used to test the normality of the data. Unpaired t-test was used to compare the data of two groups that passed normality test otherwise compared with Mann-Whitney test. For data with three or more groups, one-way ANOVA test with Tukey's test for multiple comparison was used for data that passed normality test otherwise compared with Kruskal-Wallis test followed by Dunn's test for multiple comparison. Statistically significant differences were defined as p<0.05 (*), p<0.01 (**), p<0.001 (***). The number of experiments per experiment is indicated in the legends to figures.

### Example 2

### Human Studies

This study was approved by the Institutional Review Board at the Cedars-Sinai Medical Center.

Treg Assays: Whole blood was drawn from healthy unrelated individuals to prepare peripheral blood mononuclear cells (PBMC) using the Ficoll-Hypaque gradient centrifugation method. After the stimulator PBMC was irradiated, it was exposed to the responder PBMC at 1:1 (1×10⁶/ml of each PBMCs) in the absence or presence of BTN2A2-Fc at 0, 10 mg/ml, and then incubated for 7 days for measurement of Tregs. MLR mixture were first stained with antibodies to CD45, CD3, CD4, CD25, and CD127. After permeabilization, the cells were stained with antibody to Foxp3. After acquiring cells by flow cytometry, lymphocytes separated from CD45+ leukocytes were plotted against CD4. CD4+ cells were plotted as CD25 versus CD127 and then CD25+CD127^{low/-} cells against Foxp3. CD4+/CD25+/CD127^{low-}/Foxp3+ cells were designated as Treg cells. Treg cell levels were expressed as Treg cell % in CD4+ T cells.

Th17 assays: Human PBMCs (responder cells) isolated from Ficoll-Hypaque gradient centrifugation method were cultured in irradiated stimulator cells at 1:1 (1×10⁶/ml of each PBMCs) along with Th17 differentiation cytokines cocktail (anti-CD3 antibody 0.5 ug/ml, TGFb 1.5 ng/ml, IL-6 10 ng/ml, IL-1b 10 ng/ml) with or without recombinant BTN2A2-Fc (10 mg/ml) for 5 days. Cells were stained with surface marker CD3 and CD4. After permeabilization, the cells were stained anti-RORgt antibody and analyzed for CD4+RORgt+ve co-expression using flow cytometry. Th17 cells were expressed as RORgt+ve cells % in CD4 T cells. CD45 phosphatase inhibitor was used at 125 nM concentration during the course of T cell differentiation studies.

### Example 3

### Results

### BTN2A2 Blocks CD3-Dependent Signaling in Jurkat Cells

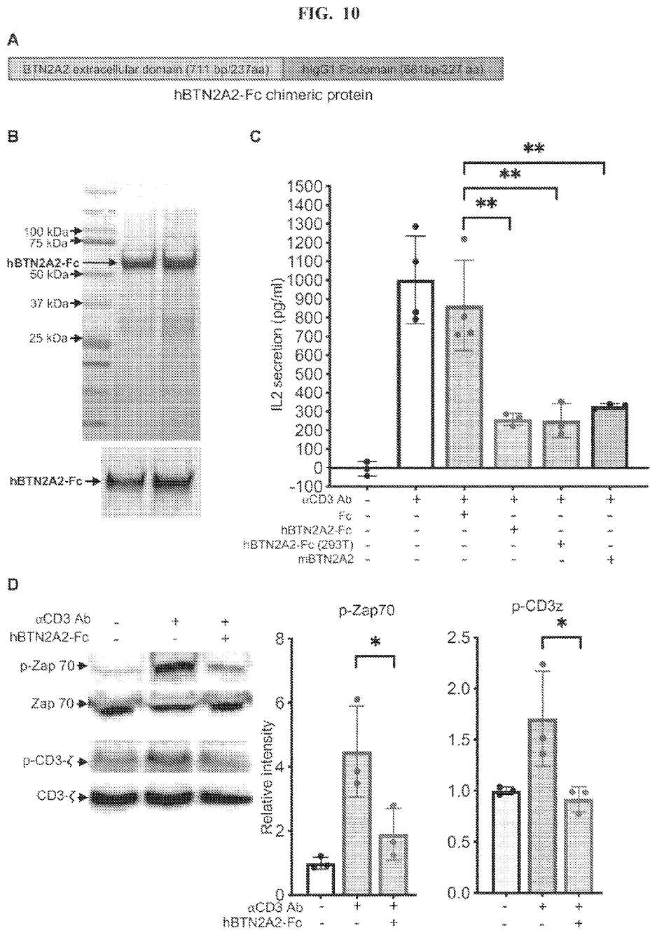

To elucidate the mechanisms underlying BTN2A2's ability to regulate T cell immunity (Ammann et al., 2013; Sarter et al., 2016), we generated a soluble recombinant human BTN2A2-Fc fusion protein using baculoviral expression systems. Consistent with prior reports performed in murine systems (Ammann et al., 2013), recombinant human BTN2A2-Fc, but not recombinant Fc control protein, blocked anti-CD3-induced IL-2 production by Jurkat cells (FIG. 10A-10C).

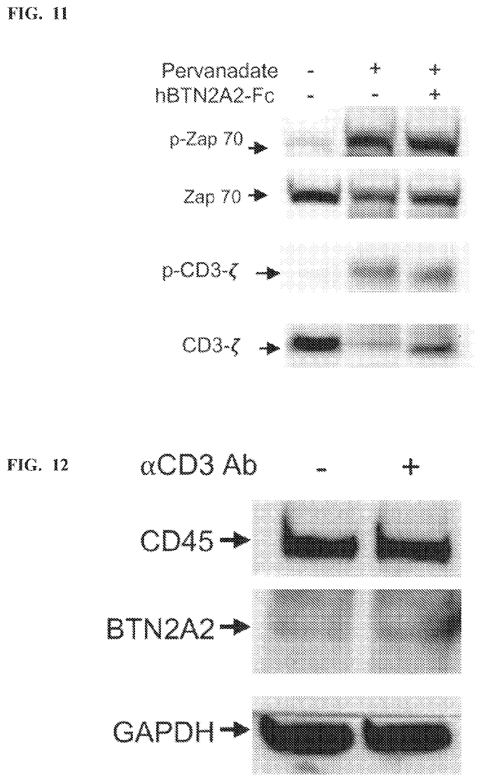

When we next evaluated signaling components downstream of the TCR, we observed that BTN2A2-Fc reduced anti-CD3 induced phosphorylation of Zap70, and CD3z in Jurkat cells (FIG. 10D). Addition of the phosphatase inhibitor pervanadate resulted in hyperphosphorylation of Zap70 and CD3z at baseline (unstimulated), and in contrast to the findings in the absence of pervanadate, addition of BTN2A2-Fe to the culture had no effect on the hyperphosphorylated proteins (FIG. 11). Together these data suggested that BTN2A2-Fc activates a phosphatase that downregulates TCR signaling.

### BTN2A2 Blocks TCR Signaling by Binding to and Enhancing CD45 Phosphatase

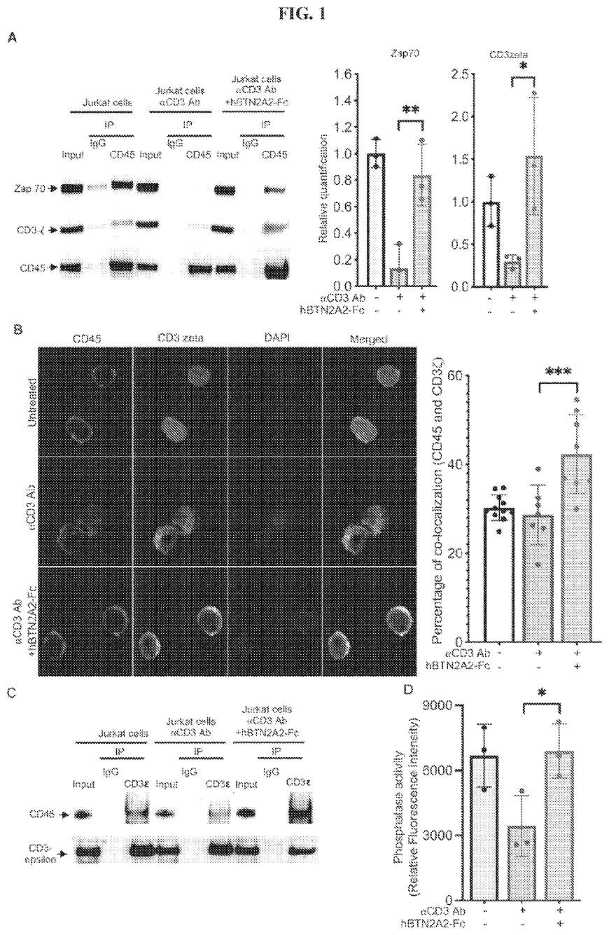

Based on the known molecular mechanisms linking CD45's phosphatase activity to TCR signaling (Jung et al., 2021; Leupin et al., 2000), we tested the hypothesis BTN2A2's inhibitory effect on TCR activation is mediated through interaction with CD45. Western blotting of CD45-associated proteins after co-immunoprecipitation (co-IP) of CD45 in unstimulated Jurkat cells demonstrated that in the absence of TCR activation, CD45 was normally associated with several components of the TCR complex (Zap70 and CD3z). However, activation of the TCR complex with anti-CD3 antibodies resulted in CD45 dissociating from Zap 70 and CD3z. In contrast, TCR activation in the presence of BTNT2A2 caused CD45 to remain associated with the TCR complex (FIG. 1A), limiting T-cell activation. Immunocytochemistry confirmed colocalization of CD45 and CD3z in untreated cells at baseline, segregation of CD45 following anti-CD3-induced TCR activation, and maintenance of CD45 and CD3z colocalization when cells were stimulated in the presence of BTN2A2, suggesting that BTN2A2 prevents the exclusion of CD45 from the immune synapse during the early phases of T cell activation (FIG. 1B).

Co-IP experiments additionally demonstrated that compared to unstimulated cells, anti-CD3 activation reduced the association of CD3e with CD45, whereas TCR activation in the presence of BTN2A2 augmented the interaction between CD45 and CD3e (FIG. 1C). Functional experiments looking at TCR-associated CD45 phosphatase activity showed that when TCR complexes were isolated by co-IP with anti-CD3e antibodies, CD45 phosphatase activity was detectable at baseline, decreased following TCR activation, and maintained when cells were activated in the presence of BTN2A2, suggesting segregation of CD45 from the TCR complex after activation and retention in the presence of BTN2A2 (Jung et al., 2021; Leupin et al., 2000) (FIG. 1D). Together with prior studies that activating CD45 phosphatase on lipid microdomains on T cell surfaces results in decreased sensitivity of TCR-mediated signaling (He et al., 2002), our new data indicate BTN2A2 dampens TCR signaling by activating CD45 phosphatase activity in the TCR complex which in turn blocks critical phosphorylation events downstream of the TCR.

BTN2A2 Interacts with CD45 Phosphatase

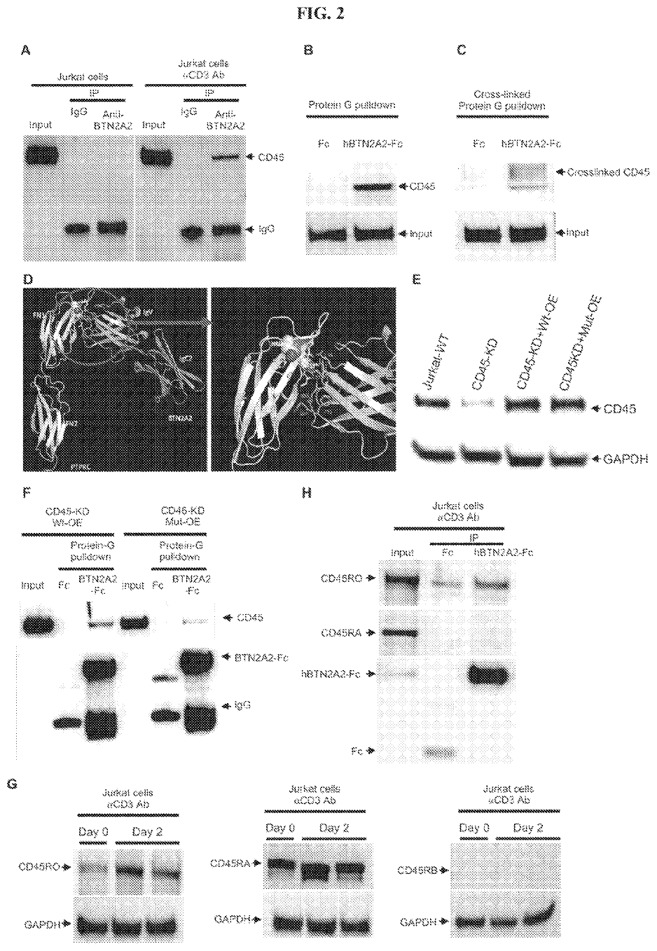

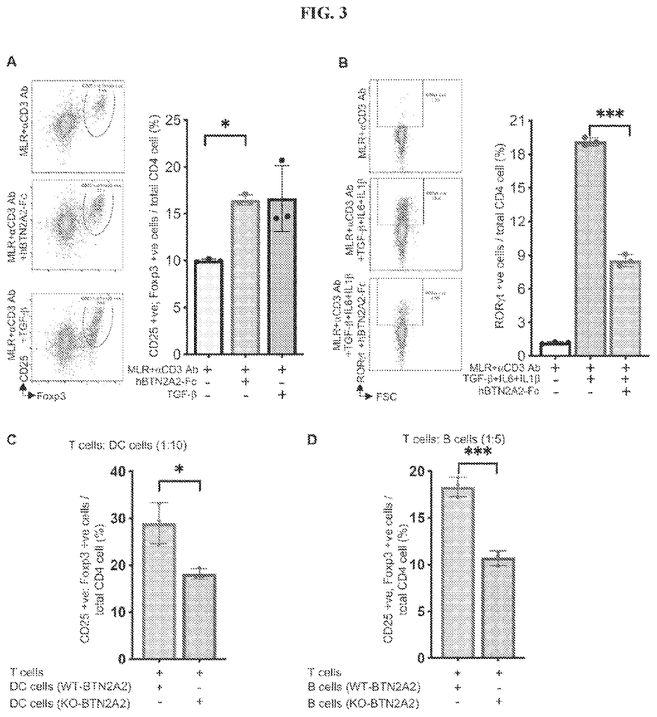

We next employed a co-IP experimental strategy using Jurkat cells to test for direct interactions between CD45 and endogenously expressed BTN2A2 (FIG. 2A). These assays showed CD45 co-immunoprecipitated with endogenous BTN2A2 in unstimulated Jurkat cells, and binding was significantly enhanced in activated cells. Anti-CD3 activation of Jurkat cells did not change endogenous CD45 and BTN2A2 expression levels (FIG. 12). Complementary experiments of cells treated with exogenous BTN2A2-Fc protein or Fc protein control confirmed that exogenous BTN2A2-Fc but not control Fe-tag protein binds to CD45 in activated T cells (FIG. 2B-C). We then performed molecular modeling to identify regions in the extracellular region of CD45 that might interact with BTN2A2. Protein homology modeling showed that BTN2A2 interacted with the extracellular fibronectin domain of CD45 protein and in particular amino acids—Asn-419 and Asn-468 (putative N-glycosylation sites based on their location within N-X-S/T consensus motifs) were critical for this interaction (FIG. 2D). We then performed site-directed mutagenesis of asparagine to alanine and confirmed that mutant CD45 interaction with BTN2A2 is significantly attenuated (FIG. 13 and FIG. 2E-F).

Based on our observations CD45 binding to endogenous BTN2A2 after activation, we suspected that BTN2A2 was preferentially binding with the CD45RO isoform, because CD45RO is predominantly expressed in activated T cells (Hermiston et al., 2003) and is upregulated after Jurkat cell activation (FIG. 2G). Considering this, we repeated the pulldown assay using isoform specific antibodies (FIG. 2H). These experiments confirmed that BTN2A2 preferentially binds CD45RO isoform, the predominant isoform in activated T cells.

### BTN2A2 Enhances Regulatory T Cell Expansion and Suppresses Th17 Cell Populations in Primary Mouse T Cells

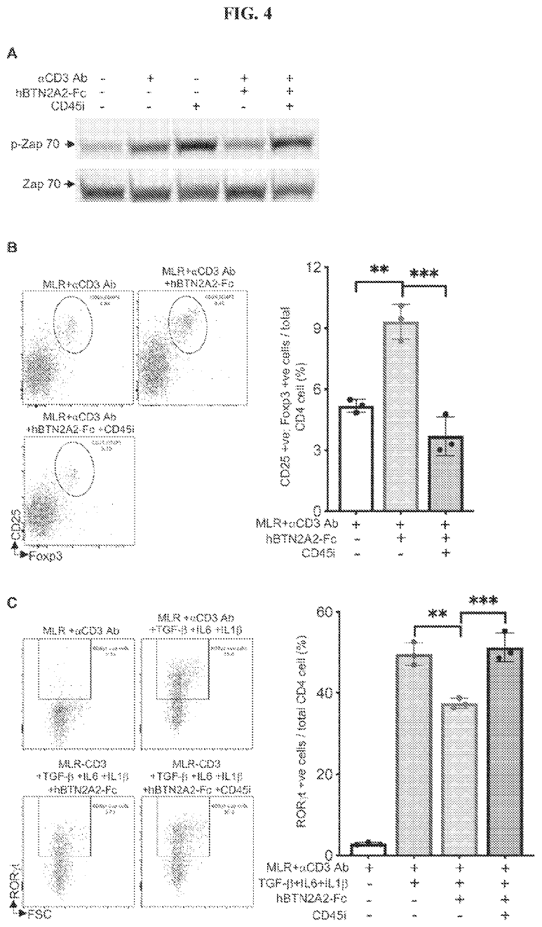

Activated CD4+ T cells differentiate into several effector cell subsets based on activation events and the cytokine milieu present during activation. Since it was previously reported that BTN2A2 induced Foxp3 expression in CD4+ T cells (Ammann et al., 2013) in short-term culture studies we tested whether T cells activated in the presence of BTN2A2 increased Treg populations in splenocytes from Foxp3-EGFP mice that co-express EGFP when Foxp3 is expressed. We cultured murine T cells (Foxp3+ reporter mice −BALB/cJ strain) in the presence of irradiated, allogeneic mouse splenocytes (CD1 strain) with or without recombinant BTN2A2-Fc. These assays showed a 50% increase in CD4+CD25+Foxp3+ T cells, in BTN2A2-Fc treated cells compared to controls, a similar magnitude to that observed in positive control cultures using exogenous TGF-β (FIG. 3A). Interestingly, short-term exposure of BTN2A2 to activated CD4+ T cells led to less proliferation and enhanced survival, phenotypes noted with a pro-differentiation state and similar to what has been reported with TGF-b (McKams and Schwartz, 2005) (FIG. 14). Parallel assays showed that recombinant BTN2A2 blocked IL-6/TGF-β/IL-1β-induced production of RORgt, the signature transcription factor of Th17 cells (FIG. 3B).

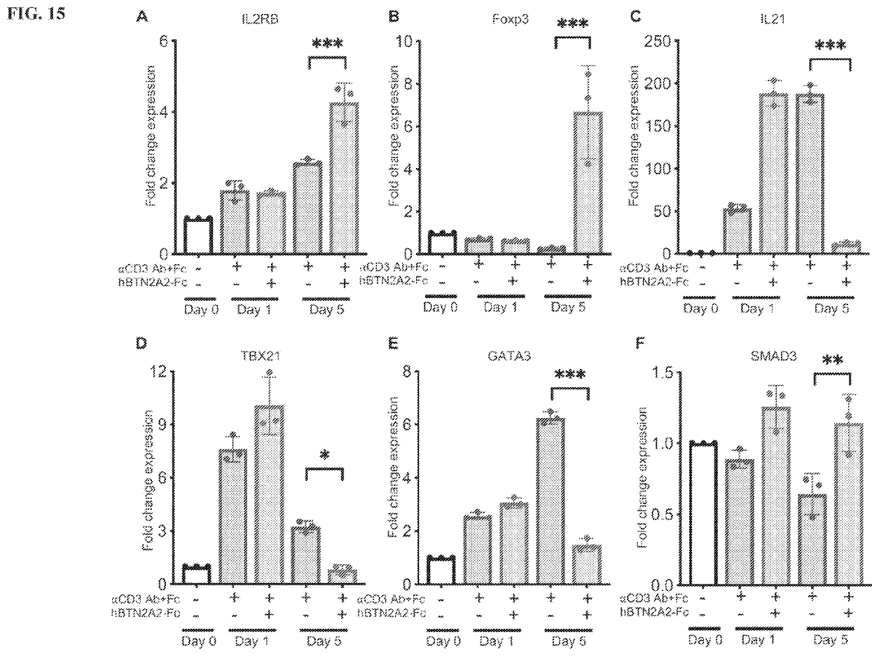

To further explore the short-term effects of BTN2A2 on T cell subset expression patterns in mixed lymphocyte reaction (MLR) culture conditions, we assessed gene expression patterns for molecules related to Th1, Th2, Treg and Th17 cells by RT-PCR. BTN2A2 upregulated IL2Rbeta and Foxp3 and downregulated IL-21 consistent with our in vitro findings of expansion of Tregs and suppression of Th17 cells (FIG. 15A-C). The assays further showed downregulation of transcription factors regulating Th1 (TBX21) and Th2 (GATA3) differentiation pathway at day 5 (FIG. 15D-F).

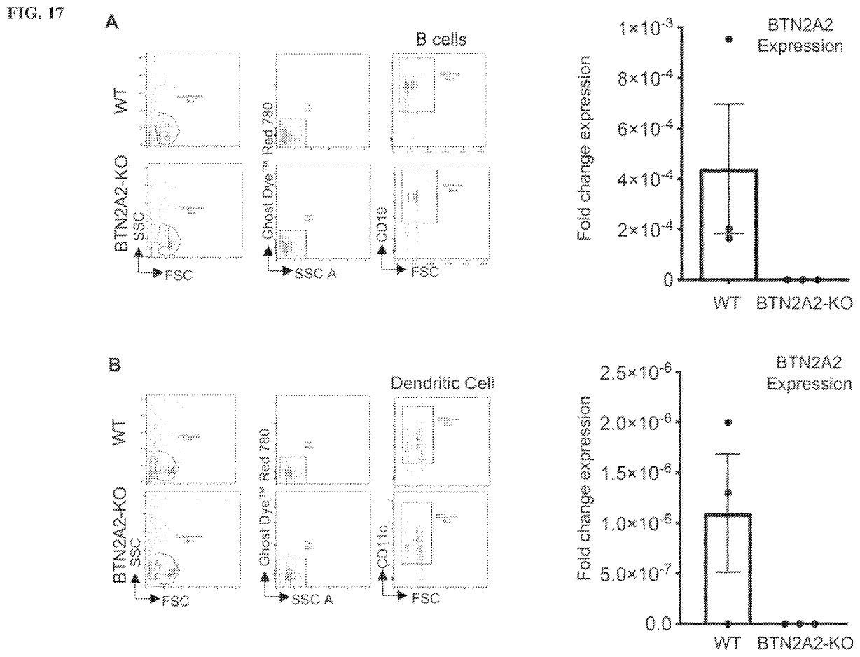

To explore the role of endogenous BTN2A2, we genetically knocked out murine BTN2A2 using CRISPR-Cas9 strategy (FIG. 16) and confirmed that expression of BTN2A2 was absent in professional APCs (CD11c+ dendritic cells and CD19+ B cells) in knock-out mice compared to wild-type littermates (FIG. 17). Interestingly, when isolated APCs from BTN2A2−/− animals were co-cultured with murine T cells from Foxp3+ reporter mice, absence of BTN2A2 from both isolated dendritic cells and isolated B cells from BTN2A2−/− animals impaired mouse T cell differentiation to Foxp3+ Tregs when compared to dendritic cells and B cells from wildtype littermates (FIG. 3C-D) BTN2A2-induced enhancement of Treg/Th17 balance is dependent on CD45 phosphatase activity.

To test whether CD45 phosphatase activity was essential for BTN2A2's actions on TCR signaling and Treg expansion, we used a previously validated CD45-specific phosphatase inhibitor (Perron et al., 2014). Addition of BTN2A2-Fc to activated Jurkat T cells decreased phosphorylation of ZAP70 kinase, while inhibition of CD45 phosphatase activity with this small molecule inhibitor (Perron et al., 2014) abrogated the effect (FIG. 4A). When we inhibited CD45 phosphatase activity during BTN2A2-Fe treatment we also observed complete abrogation of the effects of BTN2A2-Fc on increased Treg and decreased Th17 populations (FIG. 4B-C). Taken together with other experiments described above, these studies link BTN2A2 effects on CD45 phosphatase activity within the TCR complex and immune synapse and Treg and Th17 cellular balance.

### BTN2A2-Fc Therapy Exhibits Immunoregulatory Function In Vivo

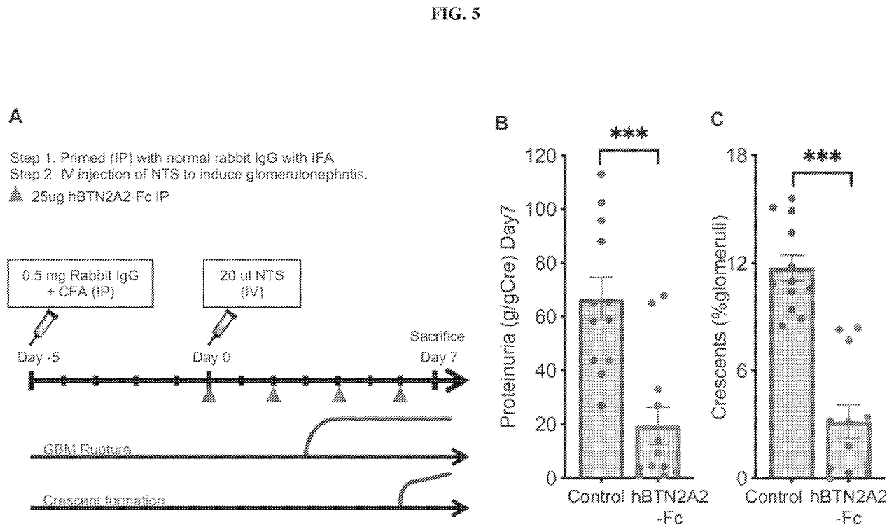

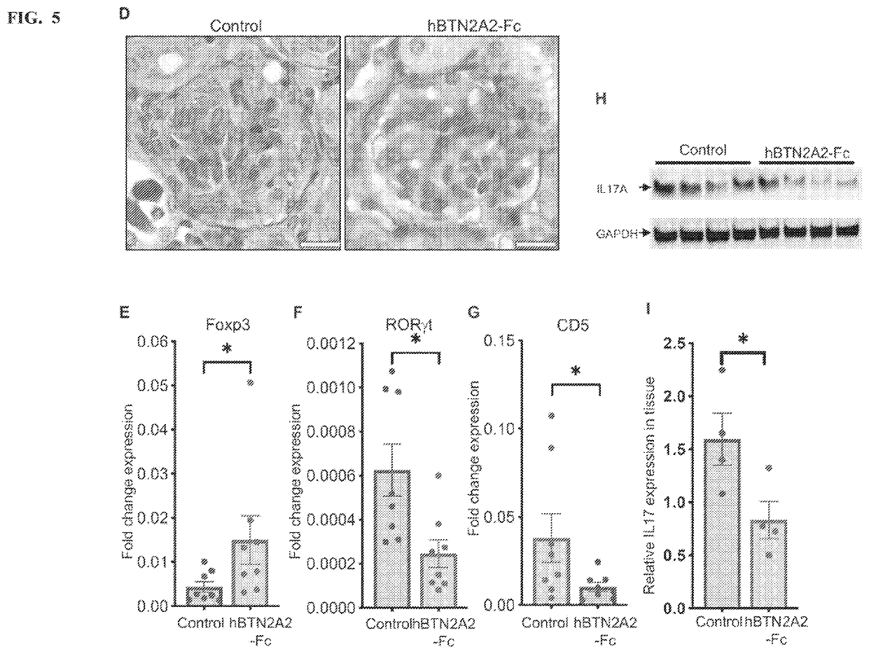

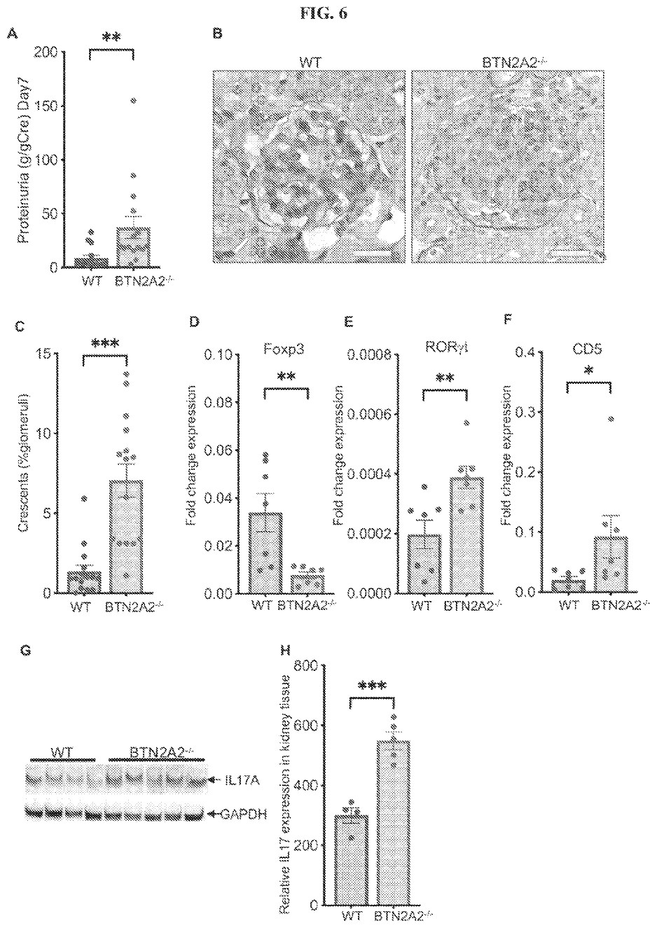

To test whether and how the in vitro observed immunoregulatory effects of BTN2A2 correlate to findings in vivo, we employed two distinct murine model systems. Consistent with previous reports (Saito et al., 2022: Tipping and Holdsworth, 2006), injection of nephrotoxic serum (NTS) in wild type B6 mice induced crescentic glomerulonephritis with significant proteinuria (FIG. 5A-D). Administration of BTN2A2-Fc during the days following NTS administration reduced proteinuria and glomerular crescent formation. Administration of BTN2A2-Fc also increased CD4+Foxp3 gene expression (FIG. 5E), decreased CD4+RORgt gene expression (FIG. 5F), attenuated T cell activation marker CD5 levels (FIG. 5G) in CD4+ T cells purified from spleen and lymph nodes. Further, BTN2A2-Fc lowered IL17A protein expression in the kidneys compared to controls (FIG. 5G-I).

We next administered a lower dose of NTS (50% less than the amount used in experiments above) to BTN2A2−/− mice (FIG. 16) and wildtype littermate controls (FIG. 6). These experiments demonstrated that lower doses of NTS induced relatively mild crescentic glomerulonephritis and proteinuria among wild-type control animals, however, in BTN2A2−/− animals we saw an exacerbation of crescentic glomerulonephritis and severe proteinuria (FIG. 6A-C). We also observed reduced Foxp3 and enhanced RORgt gene expression in splenic/lymph node CD4+ T cells from the BTN2A2−/− mice vs. controls (FIG. 6D-E). CD5 was enhanced in CD4+ T cells in BTN2A2−/− mice compared to wild type mice (FIG. 6F). Analysis of kidney tissue one week after administration of nephrotoxic serum also showed increased IL17A protein expression in the kidneys of BTN2A2−/− vs controls animals (FIG. 6G-H).

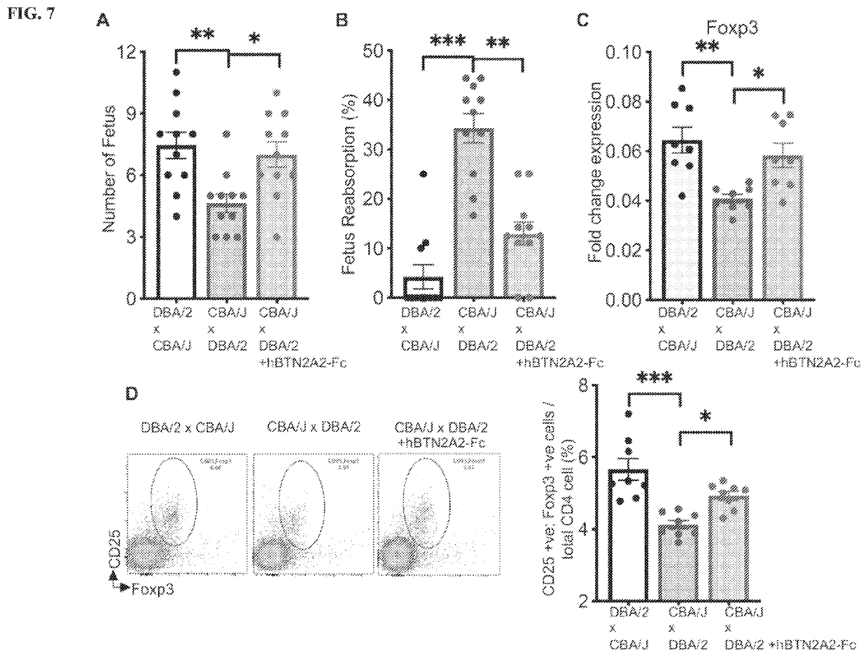

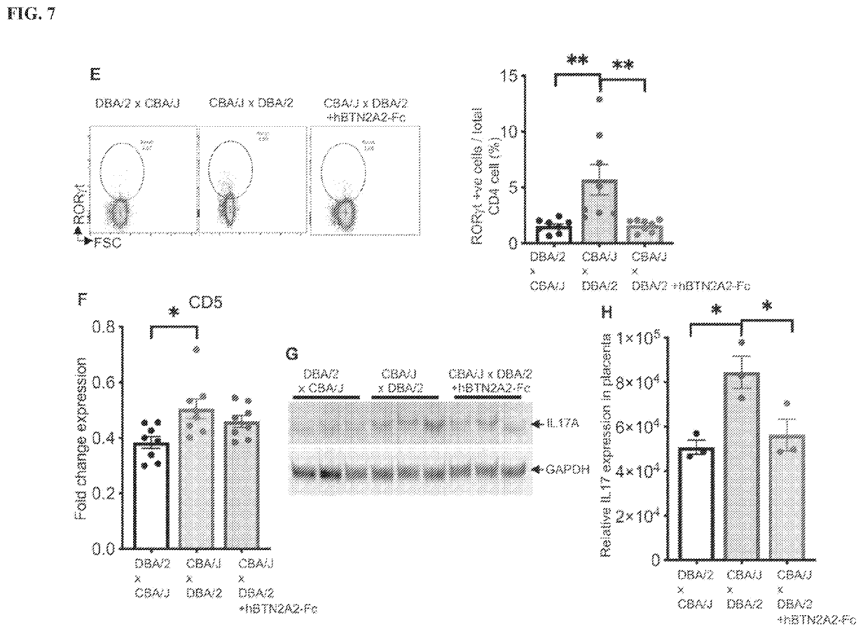

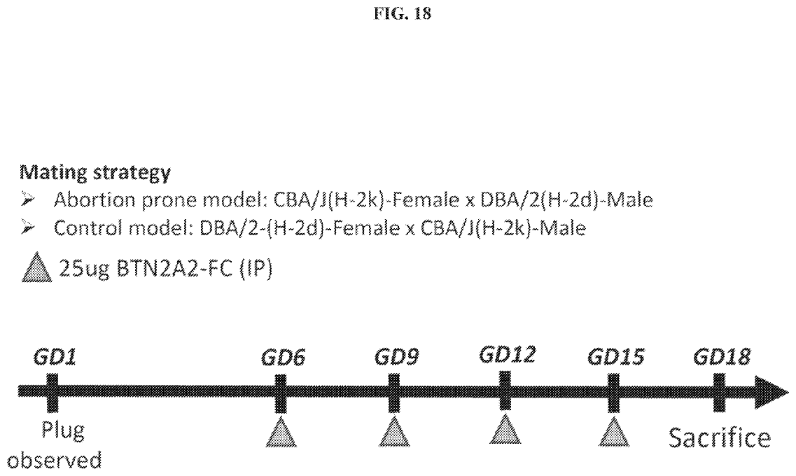

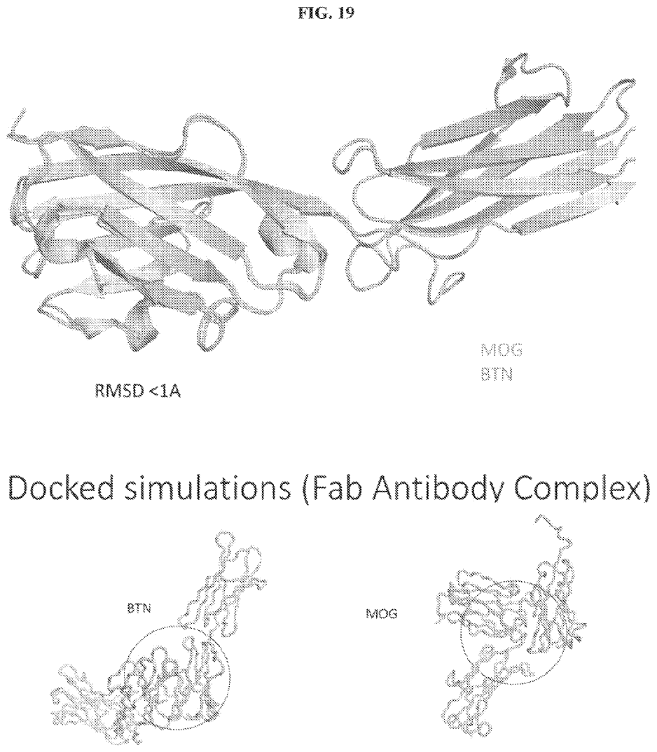

To evaluate the generalizability of the role of BTN2A2 in immunoregulation, we studied the effects of recombinant BTN2A2-Fc in DBA/2 mice mated with CBA/J mice, a model of immunologically mediated abortions (Clark et al., 2008). We confirmed that DBA/2 male×CBA/J female had reduced litter sizes and higher spontaneous adsorption rates compared to CBA/J male×DBA/2 females. Administration of BTN2A2-Fc improved litter size and rescued the excess abortion rates that were noted in the DBA/2 male×CBA/J female (FIG. 7A-B and FIG. 18). The beneficial effects of BTN2A2-Fc were associated with increased frequencies of splenic/lymph node Foxp3+ Tregs, reduced frequencies of splenic/lymph node Th17 cells and relative attenuation of CD5, consistent with our findings in the autoimmune GN model (FIG. 7D-F). In addition, BTN2A2-Fc protein also reduced placental IL-17 protein expression that correlated with improved litter sizes (FIG. 7G-H).

### BTN2A2 Enhances Treg Cell Expansion and Suppresses Th17 Cell Populations in Human PBMCs

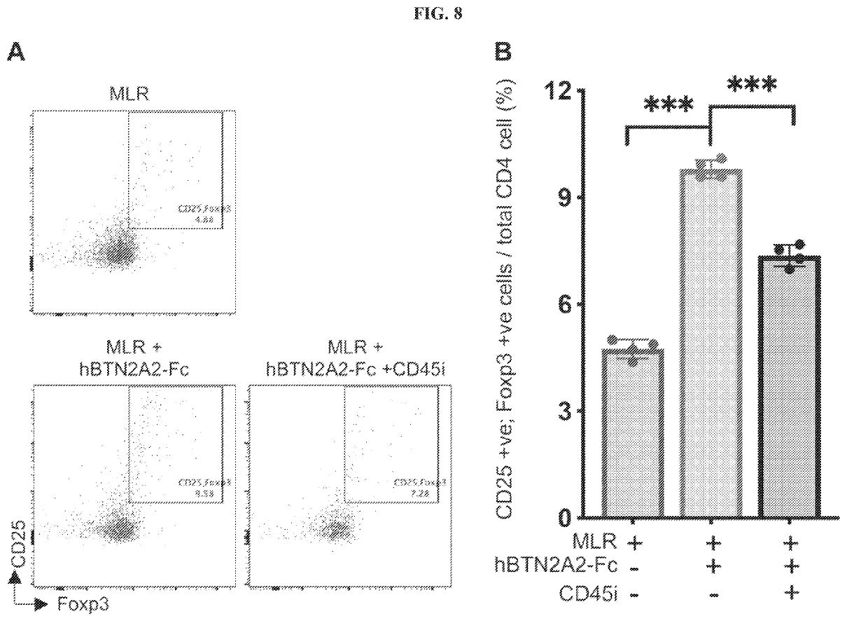

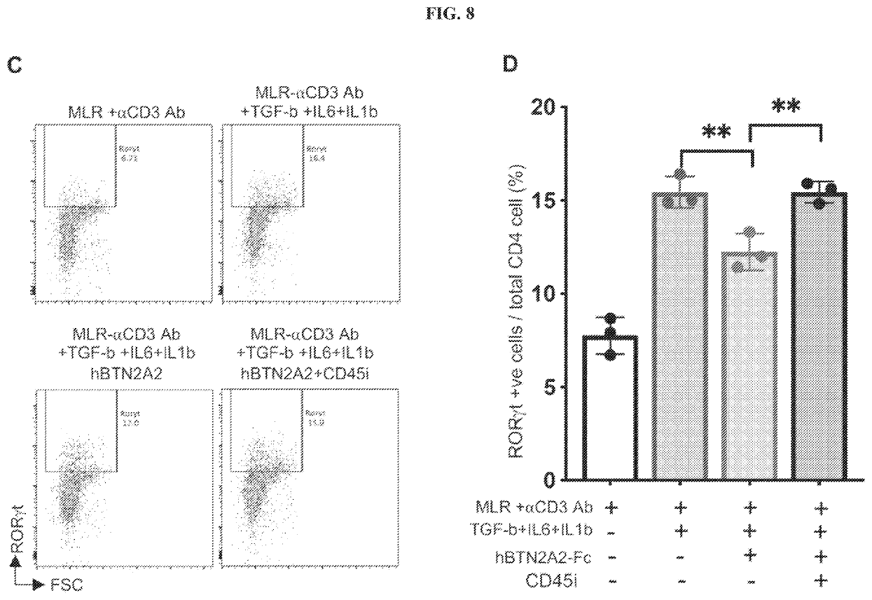

To evaluate whether the effects of recombinant BTN2A2 apply to human T cells, we analyzed human PBMCs activated by mixed lymphocyte reactions (MLR, using allogeneic stimulator cells) with and without recombinant BTN2A2-Fc, and quantified CD4+CD25+CD127^{low/-} Foxp3+ Treg cell numbers 7 days later. These co-cultures showed that the BTN2A2-Fc induced 2-fold expansion of Tregs under MLR conditions compared with controls: however BTN2A2-Fc was unable to induce Tregs in the presence of CD45 inhibitor (FIG. 8A-B). Parallel experiments revealed that recombinant BTN2A2-Fc robustly blocked the TGFb-IL6-IL1b induced increase in Th17 cells (FIG. 8C-D). Interestingly, BTN2A2-Fc was unable to block cytokines induced Th17 cells in the presence of CD45 inhibitor. Taken together, these observations suggest that CD45 phosphatase activity is necessary for BTN2A2's actions in humans as well.

Various embodiments of the invention are described above in the Detailed Description. While these descriptions directly describe the above embodiments, it is understood that those skilled in the art may conceive modifications and/or variations to the specific embodiments shown and described herein. Any such modifications or variations that fall within the purview of this description are intended to be included therein as well. Unless specifically noted, it is the intention of the inventors that the words and phrases in the specification and claims be given the ordinary and accustomed meanings to those of ordinary skill in the applicable art(s).

The foregoing description of various embodiments of the invention known to the applicant at this time of filing the application has been presented and is intended for the purposes of illustration and description. The present description is not intended to be exhaustive nor limit the invention to the precise form disclosed and many modifications and variations are possible in the light of the above teachings. The embodiments described serve to explain the principles of the invention and its practical application and to enable others skilled in the art to utilize the invention in various embodiments and with various modifications as are suited to the particular use contemplated. Therefore, it is intended that the invention not be limited to the particular embodiments disclosed for carrying out the invention.

While particular embodiments of the present invention have been shown and described, it will be obvious to those skilled in the art that, based upon the teachings herein, changes and modifications may be made without departing from this invention and its broader aspects and, therefore, the appended claims are to encompass within their scope all such changes and modifications as are within the true spirit and scope of this invention. As used herein the term “comprising” or “comprises” is used in reference to compositions, methods, and respective component(s) thereof, that are useful to an embodiment, yet open to the inclusion of unspecified elements, whether useful or not. It will be understood by those within the art that, in general, terms used herein are generally intended as “open” terms (e.g., the term “including” should be interpreted as “including but not limited to,” the term “having” should be interpreted as “having at least,” the term “includes” should be interpreted as “includes but is not limited to,” etc.). Although the open-ended term “comprising.” as a synonym of terms such as including, containing, or having, is used herein to describe and claim the invention, the present invention, or embodiments thereof may alternatively be described using alternative terms such as “consisting of” or “consisting essentially of.”

Unless stated otherwise, the terms “a” and “an” and “the” and similar references used in the context of describing a particular embodiment of the application (especially in the context of claims) may be construed to cover both the singular and the plural. The recitation of ranges of values herein is merely intended to serve as a shorthand method of referring individually to each separate value falling within the range. Unless otherwise indicated herein, each individual value is incorporated into the specification as if it were individually recited herein. All methods described herein may be performed in any suitable order unless otherwise indicated herein or otherwise clearly contradicted by context. The use of any and all examples, or exemplary language (for example, “such as”) provided with respect to certain embodiments herein is intended merely to better illuminate the application and does not pose a limitation on the scope of the application otherwise claimed. The abbreviation. “e.g.” is derived from the Latin exempli gratia, and is used herein to indicate a non-limiting example. Thus, the abbreviation “e.g.” is synonymous with the term “for example.” No language in the specification should be construed as indicating any non-claimed element essential to the practice of the application.

“Optional” or “optionally” means that the subsequently described circumstance may or may not occur, so that the description includes instances where the circumstance occurs and instances where it does not.

Groupings of alternative elements or embodiments of the present disclosure disclosed herein are not to be construed as limitations. Each group member may be referred to and claimed individually or in any combination with other members of the group or other elements found herein. One or more members of a group may be included in, or deleted from, a group for reasons of convenience and/or patentability. When any such inclusion or deletion occurs, the specification is herein deemed to contain the group as modified thus fulfilling the written description of all Markush groups used in the appended claims.

What is claimed is:

1. A method of reducing CD3-dependent T cell signaling in a subject in need thereof, comprising
administering butyrophilin A2 (BTN2A2), fragment thereof, a BTN2A2-related isoform, a BTN2A2-related isoform fragment or a fusion polypeptide comprising any of the foregoing, to the subject.

2. A method of increasing T-regulatory (Treg) cells, or decreasing T-helper 17 (Th17) cells, or both in a subject in need thereof, comprising:
administering butyrophilin A2 (BTN2A2), fragment thereof, a BTN2A2-related isoform, a BTN2A2-related isoform fragment, or a fusion polypeptide comprising any of the foregoing, to the subject.

3. The method of claim 1 or 2, wherein the BTN2A2, fragment thereof, BTN2A2-related isoform, BTN2A2-related isoform fragment or fusion polypeptide comprises human BTNA2 or a fragment or isoform thereof.

4. The method of any of the previous claims, wherein the subject is pregnant, and the method inhibits or reduces the likelihood of miscarriage of a fetus by increasing Treg cells.

5. The method of any of the previous claims, wherein the subject has renal damage or a condition associated with renal damage.

6. The method of any of the previous claims, wherein the subject is an organ, tissue or cell transplant recipient.

7. The method of claim 6, wherein the method reduces the likelihood of organ, tissue or cell transplant rejection and/or an autoimmune or inflammatory reaction.

8. The method of any of the previous claims, wherein the method reduces a need for an immunosuppressive therapy or reduces the amount of immunosuppressive therapy administered to the subject.

9. The method of claim 8, wherein the method reduces or eliminates the otherwise toxic effects of the immunosuppressive therapy in the subject.

10. The method of any of the previous claims, wherein the subject has an autoimmune or inflammatory disorder or a disorder characterized by the accumulation of pathologic Th1/Th17 cells.

11. The method of claim 10, wherein the autoimmune or inflammatory disorder or disorder characterized by the accumulation of pathologic Th1/Th17 cells comprises allergy, asthma, glomerulonephritis, inflammatory bowel disease or Crohn's disease, rheumatoid arthritis, an autoimmune neurological disease, antibody mediated transplant rejection, infantile cholestasis, haemophagocytic lymphohistiocytosis, erythrocytic haemophagocytosis, malnutrition, systemic lupus erythematosus (lupus), sarcoidosis, psoriasis, myasthenia gravis or HIV.

12. The method of claim 11, wherein the autoimmune neurological disease is multiple sclerosis or myelin oligodendrocyte glycoprotein (MOG) antibody disease or Alzheimer's disease.

13. A method of treating a disease or condition in a subject in need thereof, comprising:
administering butyrophilin A2 (BTN2A2), a BTN2A2 fragment thereof, a BTN2A2-related isoform, or a BTN2A2-related isoform fragment, to the subject.

14. The method of claim 13, wherein disease or condition is an autoimmune disorder or inflammatory disorder.

15. The method of claim 14, wherein the autoimmune or inflammatory disorder comprises allergy, asthma, glomerulonephritis, inflammatory bowel disease or Crohn's disease, rheumatoid arthritis, an autoimmune neurological disease, antibody mediated transplant rejection, infantile cholestasis, haemophagocytic lymphohistiocytosis, erythrocytic haemophagocytosis, malnutrition, systemic lupus erythematosus (lupus), sarcoidosis, psoriasis, myasthenia gravis or HIV.

16. The method of claim 15, wherein the autoimmune neurological disease is multiple sclerosis or myelin oligodendrocyte glycoprotein (MOG) antibody disease or Alzheimer's disease.

17. The method of claim 13, wherein the disease or condition is associated with renal damage.

18. The method of claim 13, wherein disease or condition is organ transplant rejection and the method reduces the likelihood of the organ transplant rejection.

19. The method of claim 13, wherein the disease or condition is organ transplant rejection and the method reduces a need for an immunosuppressive therapy or reducing the amount of immunosuppressive therapy needed by the subject.

20. The method of claim 19, wherein the toxic effects of the immunosuppressive therapy are reduced or eliminated in the subject.

21. The method of any one of the previous claims, wherein the butyrophilin A2 (BTN2A2) or the BTN2A2 fragment thereof is linked to an albumin, optionally human serum albumin.

22. The method of any one of the previous claims, wherein the butyrophilin A2 (BTN2A2) or the BTN2A2 fragment thereof is linked or fused to a Fc domain or a fragment of the Fc domain (“BTN2A2-Fc fusion protein”) or wherein the BTN2A2-related isoform or the BTN2A2-related isoform fragment is linked or fused to the Fc domain or the fragment of the Fc domain (“BTN2A2-related isoform-Fc fusion protein”), optionally an IgG1, IgG2, IgG3 or IgG4 Fe, which optionally may be mutated, further optionally mutated in order to enhance or reduce an Fc-associated effector function, further optionally any of the mutations shown in Table 3.

23. The method of claim 22, wherein the BTN2A2 or the BTN2A2 fragment thereof is linked to a Fc domain or a fragment of the Fc domain by a linker, optionally a peptide of 2-50 amino acids, or wherein the BTN2A2-related isoform or the BTN2A2-related isoform fragment is linked to the Fc domain or the fragment of the Fc domain by a linker, and the linker is G, polyserine, polyglycine, glycine-serine, GGGGSn (SEQ ID NO:6)n. GGGGGSn (SEQ ID NO:7)n, leucine zipper, r aliphatic, or helical peptides.

24. The method of any one of the previous claims, wherein the butyrophilin A2 (BTN2A2) or the BTN2A2 fragment thereof, the BTN2A2-related isoform or the BTN2A2-related isoform fragment is modified by glycosylation or PEGylation or lipidation or by attachment to cholesterol.

25. The method of any one of the previous claims, wherein the BTN2A2 fragment comprises an extracellular domain of BTN2A2.

26. The method of any one of the previous claims, wherein the BTN2A2 fragment does not comprise a transmembrane domain, a cytoplasmic domain, or both.

27. The method of any one of the previous claims, wherein the BTN2A2-related isoform is BTN1A1, B2N2A1, BTN3A1, BTN3A2 or BTN3A3.

28. The method of any one of the previous claims wherein the BTN2A2-related isoform fragment does not comprise the transmembrane domain, the cytoplasmic domain, or both.

29. A fusion polypeptide, comprising:
a butyrophilin A2 (BTN2A2), a BTN2A2 fragment, a BTN2A2-related isoform, or a BTN2A2-related isoform fragment or a combination thereof; and
an Fc domain or a fragment of the Fc domain, optionally an IgG1, IgG2, IgG3 or IgG4 Fc, which optionally may be mutated, further optionally mutated in order to enhance or reduce an Fc-associated effector function, further optionally any of the mutations shown in Table 3.

30. The fusion polypeptide of claim 29, further comprising a linker between the BTN2A2, BTN2A2 fragment, BTN2A2-related isoform, BTN2A2-related isoform fragment, and the Fc domain or the fragment of the Fc domain, optionally an IgG1, IgG2, IgG3 or IgG4 Fc, which optionally may be mutated, further optionally mutated in order to enhance or reduce an Fc-associated effector function, further optionally any of the mutations shown in Table 3.

31. The fusion protein of claim 30, wherein the Fc domain or the fragment of the Fc domain is from an IgG1 antibody.

32. The fusion protein of claims 29 or 31, wherein the Fc domain or the fragment of the Fc domain comprises one or more mutations.

33. The fusion protein of claim 32, wherein the mutation is Met208Leu, Asn214Ser, or both, in reference to SEQ ID NO:4.

34. The fusion protein of any one of claims 29-33, further comprising a spacer peptide between the BTN2A2, BTN2A2 fragment, the BTN2A2-related isoform, or the BTN2A2-related isoform fragment, and the Fc domain or the fragment of the Fc domain.

35. The fusion protein of any one of claims 29-34, wherein the spacer peptide is selected from the group consisting of IEGRMDDISSTMVRS (SEQ ID NO:56), IEGRMD (SEQ ID NO:57), EAEAEAK (SEQ ID NO:58), SIINFEKL (SEQ ID NO:59), and GGGS (SEQ ID NO:60).

36. The fusion protein of any one of claims 29-35, wherein the BTN2A2 fragment comprises the extracellular domain of BTN2A2.

37. The fusion protein of any one of claims 29-33, wherein the BTN2A2 fragment does not comprise the transmembrane domain, the cytoplasmic domain, or both.

38. The fusion protein of any one of claims 29-35, wherein the BTN2A2-related isoform is BTN1A1, B2N2A1, BTN3A1, BTN3A2 or BTN3A3.

39. The fusion protein of any one of claim 29-35 and 38, wherein the BTN2A2-related isoform fragment does not comprise the transmembrane domain, the cytoplasmic domain, or both.

40. A modified polypeptide, comprising:
a butyrophilin A2 (BTN2A2), a BTN2A2 fragment, a BTN2A2-related isoform, or a BTN2A2-related isoform fragment or a combination thereof,
wherein the butyrophilin A2 (BTN2A2), the BTN2A2 fragment, the BTN2A2-related isoform, or the BTN2A2-related isoform fragment, or the combination thereof is PEGylated or glycosylated.

41. The modified polypeptide claim 40, wherein the BTN2A2 fragment comprises the extracellular domain of BTN2A2.

42. The modified polypeptide of one of claims 40 or 41, wherein the BTN2A2 fragment does not comprise the transmembrane domain, the cytoplasmic domain, or both.

43. The modified polypeptide of one of claims 41 or 42, wherein the BTN2A2 fragment binds to CD45.

44. The modified polypeptide of claim 4040, wherein the BTN2A2-related isoform is BTN1A1, B2N2A1, BTN3A1, BTN3A2 or BTN3A3.

45. The modified polypeptide of claim 40 and 44, wherein the BTN2A2-related isoform fragment does not comprise the transmembrane domain, the cytoplasmic domain, or both.

46. An immunoassay, comprising:
an anti-butyrophilin A2 (BTN2A2) antibody, an anti-BTN2A2 fragment antibody, an anti-BTN2A2-related isoform antibody, or an anti-BTN2A2-related isoform fragment antibody, or a combination thereof immobilized on a solid support; and
the solid support.

47. The assay of claim 46, further comprising a biological sample obtained from a subject.

48. A method of using the assay of claim 43 for patient monitoring, comprising:
contacting a biological sample obtained from a patient to the assay of claim 43; and
detecting the level of butyrophilin A2 (BTN2A2), the BTN2A2 fragment, the BTN2A2-related isoform, or the BTN2A2-related isoform fragment, or the combination thereof.

49. The method of claim 48, further comprising selecting or administrating a treatment selected from
(i) butyrophilin A2 (BTN2A2), a BTN2A2 fragment thereof, a BTN2A2-related isoform, or a BTN2A2-related isoform fragment:
(ii) a fusion polypeptide, comprising:
a butyrophilin A2 (BTN2A2), a BTN2A2 fragment, a BTN2A2-related isoform, or a BTN2A2-related isoform fragment or a combination thereof; and an Fc domain or a fragment of the Fc domain;
(iii) a modified polypeptide, comprising:
a butyrophilin A2 (BTN2A2), a BTN2A2 fragment, a BTN2A2-related isoform, or a BTN2A2-related isoform fragment or a combination thereof, wherein the butyrophilin A2 (BTN2A2), the BTN2A2 fragment, the BTN2A2-related isoform, or the BTN2A2-related isoform fragment, or the combination thereof is PEGylated or glycosylated; or
(iv) a non-BTN2A2 related treatment.

50. A method of using the assay of claim 46 for patient stratification, comprising:
contacting a biological sample obtained from a patient to the assay of claim 43:
detecting the level of butyrophilin A2 (BTN2A2), the BTN2A2 fragment, the BTN2A2-related isoform, or the BTN2A2-related isoform fragment, or the combination thereof; and
stratifying the patient for treatment with
(i) butyrophilin A2 (BTN2A2), a BTN2A2 fragment thereof, a BTN2A2-related isoform, or a BTN2A2-related isoform fragment;
(ii) a fusion polypeptide, comprising:
a butyrophilin A2 (BTN2A2), a BTN2A2 fragment, a BTN2A2-related isoform, or a BTN2A2-related isoform fragment or a combination thereof, and an Fc domain or a fragment of the Fc domain;
(iii) a modified polypeptide, comprising:
a butyrophilin A2 (BTN2A2), a BTN2A2 fragment, a BTN2A2-related isoform, or a BTN2A2-related isoform fragment or a combination thereof, wherein the butyrophilin A2 (BTN2A2), the BTN2A2 fragment, the BTN2A2-related isoform, or the BTN2A2-related isoform fragment, or the combination thereof is PEGylated or glycosylated: or
(iv) a non-BTN2A2 related treatment.
